SEQUENCE LISTING (38 sequences)

SEQ ID NO: 1 - DNA, 1277 bp, Gallus gallus
atggtcgaaatgctgctgttgacaagaattctcttggtgggcttcatctgcgctcttttagtctcctctgggctgacttgtggaccaggcaggggcattggaaaaaggaggcaccccaaaaagctgaccccgttagcctataagcagtttattcccaatgtggcagagaagaccctaggggccagtggaagatatgaagggaagatcacaagaaactccgagagatttaaagaactaaccccaaattacaaccctgacattatttttaaggatgaagagaacacgggagctgacagactgatgactcagcgctgcaaggacaagctgaatgccctggcgatctcggtgatgaaccagtggcccggggtgaagctgcgggtgaccgagggctgggacgaggatggccatcactccgaggaatcgctgcactacgagggtcgcgccgtggacatcaccacgtcggatcgggaccgcagcaagtacggaatgctggcccgcctcgccgtcgaggccggcttcgactgggtctactacgagtccaaggcgcacatccactgctccgtcaaagcagaaaactcagtggcagcgaaatcaggaggctgcttccctggctcagccacagtgcacctggagcatggaggcaccaagctggtgaaggacctgagccctggggaccgcgtgctggctgctgacgcggacggccggctgctctacagtgacttcctcaccttcctcgaccggatggacagctcccgaaagctcttctacgtcatcgagacgcggcagccccgggcccggctgctactgacggcggcccacctgctctttgtggccccccagcacaaccagtcggaggccacagggtccaccagtggccaggcgctcttcgccagcaacgtgaagcctggccaacgtgtctatgtgctgggcgagggcgggcagcagctgctgccggcgtctgtccacagcgtctcattgcgggaggaggcgtccggagcctacgccccactcaccgcccagggcaccatcctcatcaaccgggtgttggcctcctgctacgccgtcatcgaggagcacagttgggcccattgggccttcgcaccattccgcttggctcaggggctgctggccgccctctgcccagatggggccatccctactgccgccaccaccaccactggcatccattggtactcacggctcctctaccgcatcggcagctgggtgctggatggtgacgcgctgcatccgctgggcatggtggcaccggccagctg

SEQ ID NO: 2 - DNA, 1190 bp, Mus musculus
atggctctgccggccagtctgttgcccctgtgctgcttggcactcttggcactatctgcccagagctgcgggccgggccgaggaccggttggccggcggcgttatgtgcgcaagcaacttgtgcctctgctatacaagcagtttgtgcccagtatgcccgagcggaccctgggcgcgagtgggccagcggaggggagggtaacaagggggtcggagcgcttccgggacctcgtacccaactacaaccccgacataatcttcaaggatgaggagaacagcggcgcagaccgcctgatgacagagcgttgcaaagagcgggtgaacgctctagccatcgcggtgatgaacatgtggcccggagtacgcctacgtgtgactgaaggctgggacgaggacggccaccacgcacaggattcactccactacgaaggccgtgccttggacatcaccacgtctgaccgtgaccgtaataagtatggtttgttggcgcgcctagctgtggaagccggattcgactgggtctactacgagtcccgcaaccacatccacgtatcggtcaaagctgataactcactggcggtccgagccggaggctgctttccgggaaatgccacggtgcgcttgcggagcggcgaacggaaggggctgagggaactacatcgtggtgactgggtactggccgctgatgcagcgggccgagtggtacccacgccagtgctgctcttcctggaccgggatctgcagcgccgcgcctcgttcgtggctgtggagaccgagcggcctccgcgcaaactgttgctcacaccctggcatctggtgttcgctgctcgcgggccagcgcctgctccaggtgactttgcaccggtgttcgcgcgccgcttacgtgctggcgactcggtgctggctcccggcggggacgcgctccagccggcgcgcgtagcccgcgtggcgcgcgaggaagccgtgggcgtgttcgcaccgctcactgcgcacgggacgctgctggtcaacgacgtcctcgcctcctgctacgcggttctagagagtcaccagtgggcccaccgcgccttcgcccctttgcggctgctgcacgcgctcggggctctgctccctgggggtgcagtccagccgactggcatgcattggtactctcgcctcctttaccgcttggccgaggagttaatgggctg

SEQ ID NO: 3 - DNA, 1281 bp, Mus musculus
atgtctcccgcctggctccggccccgactgcggttctgtctgttcctgctgctgctgcttctggtgccggcggcgcggggctgcgggccgggccgggtggtgggcagccgccggaggccgcctcgcaagctcgtgcctcttgcctacaagcagttcagccccaacgtgccggagaagaccctgggcgccagcgggcgctacgaaggcaagatcgcgcgcagctctgagcgcttcaaagagctcacccccaactacaatcccgacatcatcttcaaggacgaggagaacacgggtgccgaccgcctcatgacccagcgctgcaaggaccgtctgaactcactggccatctctgtcatgaaccagtggcctggtgtgaaactgcgggtgaccgaaggccgggatgaagatggccatcactcagaggagtctttacactatgagggccgcgcggtggatatcaccacctcagaccgtgaccgaaataagtatggactgctggcgcgcttagcagtggaggccggcttcgactgggtgtattacgagtccaaggcccacgtgcattgctctgtcaagtctgagcattcggccgctgccaagacaggtggctgctttcctgccggagcccaggtgcgcctagagaacggggagcgtgtggccctgtcagctgtaaagccaggagaccgggtgctggccatgggggaggatgggacccccaccttcagtgatgtgcttattttcctggaccgcgagccaaaccggctgagagctttccaggtcatcgagactcaggatcctccgcgtcggctggcgctcacgcctgcccacctgctcttcattgcggacaatcatacagaaccagcagcccacttccgggccacatttgccagccatgtgcaaccaggccaatatgtgctggtatcaggggtaccaggcctccagcctgctcgggtggcagctgtctccacccacgtggcccttgggtcctatgctcctctcacaaggcatgggacacttgtggtggaggatgtggtggcctcctgctttgcagctgtggctgaccaccatctggctcagttggccttctggcccctgcgactgtttcccagtttggcatggggcagctggaccccaagtgagggtgttcactcctaccctcagatgctctaccgcctggggcgtctcttgctagaagagagcaccttccatccactgggcatgtctggggcaggaagctgaagggactctaaccactgccctcctggaactgctgtgcgtggatcc

SEQ ID NO: 4 - DNA, 1313 bp, Mus musculus
atgctgctgctgctggccagatgttttctggtgatccttgcttcctcgctgctggtgtgccccgggctggcctgtgggcccggcagggggtttggaaagaggcggcaccccaaaaagctgacccctttagcctacaagcagtttattcccaacgtagccgagaagaccctaggggccagcggcagatatgaagggaagatcacaagaaactccgaacgatttaaggaactcacccccaattacaaccccgacatcatatttaaggatgaggaaaacacgggagcagaccggctgatgactcagaggtgcaaagacaagttaaatgccttggccatctctgtgatgaaccagtggcctggagtgaggctgcgagtgaccgagggctgggatgaggacggccatcattcagaggagtctctacactatgagggtcgagcagtggacatcaccacgtccgaccgggaccgcagcaagtacggcatgctggctcgcctggctgtggaagcaggtttcgactgggtctactatgaatccaaagctcacatccactgttctgtgaaagcagagaactccgtggcggccaaatccggcggctgtttcccgggatccgccaccgtgcacctggagcagggcggcaccaagctggtgaaggacttacgtcccggagaccgcgtgctggcggctgacgaccagggccggctgctgtacagcgacttcctcaccttcctggaccgcgacgaaggcgccaagaaggtcttctacgtgatcgagacgctggagccgcgcgagcgcctgctgctcaccgccgcgcacctgctcttcgtggcgccgcacaacgactcggggcccacgcccgggccaagcgcgctctttgccagccgcgtgcgccccgggcagcgcgtgtacgtggtggctgaacgcggcggggaccgccggctgctgcccgccgcggtgcacagcgtgacgctgcgagaggaggaggcgggcgcgtacgcgccgctcacggcgcacggcaccattctcatcaaccgggtgctcgcctcgtgctacgctgtcatcgaggagcacagctgggcacaccgggccttcgcgcctttccgcctggcgcacgcgctgctggccgcgctggcacccgcccgcacggacggcgggggcgggggcagcatccctgcagcgcaatctgcaacggaagcgaggggcgcggagccgactgcgggcatccactggtactcgcagctgctctaccacattggcacctggctgttggacagcgagaccatgcatcccttgggaatggcggtcaagtccagctg

SEQ ID NO: 5 - DNA, 1256 bp, Brachydanio rerio
atgcggcttttgacgagagtgctgctggtgtctcttctcactctgtccttggtggtgtccggactggcctgcggtcctggcagaggctacggcagaagaagacatccgaagaagctgacacctctcgcctacaagcagttcatacctaatgtcgcggagaagaccttaggggccagcggcagatacgagggcaagataacgcgcaattcggagagatttaaagaacttactccaaattacaatcccgacattatctttaaggatgaggagaacacgggagcggacaggctcatgacacagagatgcaaagacaagctgaactcgctggccatctctgtaatgaaccactggccaggggttaagctgcgtgtgacagagggctgggatgaggacggtcaccattttgaagaatcactccactacgagggaagagctgttgatattaccacctctgaccgagacaagagcaaatacgggacactgtctcgcctagctgtggaggctggatttgactgggtctattacgagtccaaagcccacattcattgctctgtcaaagcagaaaattcggttgctgcgaaatctgggggctgtttcccaggttcggctctggtctcgctccaggacggaggacagaaggccgtgaaggacctgaaccccggagacaaggtgctggcggcagacagcgcgggaaacctggtgttcagcgacttcatcatgttcacagaccgagactccacgacgcgacgtgtgttttacgtcatagaaacgcaagaacccgttgaaaagatcaccctcaccgccgctcacctcctttttgtcctcgacaactcaacggaagatctccacaccatgaccgccgcgtatgccagcagtgtcagagccggacaaaaggtgatggttgttgatgatagcggtcagcttaaatctgtcatcgtgcagcggatatacacggaggagcagcggggctcgttcgcaccagtgactgcacatgggaccattgtggtcgacagaatactggcgtcctgttacgccgtaatagaggaccaggggcttgcgcatttggccttcgcgcccgccaggctctattattacgtgtcatcattcctgtcccccaaaactccagcagtcggtccaatgcgactttacaacaggagggggtccactggtactccaggctcctgtcatcaaatgggaacgtggcttttggacagcaacatgcttcatcctttggggatgtcagtaaactcaagctg

SEQ ID NO: 6 - DNA, 1425 bp, Homo sapiens
  modified_base (1387...1389): n=a, c, g, or t
atgctgctgctggcgagatgtctgctgctagtcctcgtctcctcgctgctggtatgctcgggactggcgtgcggaccgggcagggggttcgggaagaggaggcaccccaaaaagctgacccctttagcctacaagcagtttatccccaatgtggccgagaagaccctaggcgccagcggaaggtatgaagggaagatctccagaaactccgagcgatttaaggaactcacccccaattacaaccccgacatcatatttaaggatgaagaaaacaccggagcggacaggctgatgactcagaggtgtaaggacaagttgaacgctttggccatctcggtgatgaaccagtggccaggagtgaaactgcgggtgaccgagggctgggacgaagatggccaccactcagaggagtctctgcactacgagggccgcgcagtggacatcaccacgtctgaccgcgaccgcagcaagtacggcatgctggcccgcctggcggtggaggccggcttcgactgggtgtactacgagtccaaggcacatatccactgctcggtgaaagcagagaactcggtggcggccaaatcgggaggctgcttcccgggctcggccacggtgcacctggagcagggcggcaccaagctggtgaaggacctgagccccggggaccgcgtgctggcggcggacgaccagggccggctgctctacagcgacttcctcactttcctggaccgcgacgacggcgccaagaaggtcttctacgtgatcgagacgcgggagccgcgcgagcgcctgctgctcaccgccgcgcacctgctctttgtggcgccgcacaacgactcggccaccggggagcccgaggcgtcctcgggctcggggccgccttccgggggcgcactggggcctcgggcgctgttcgccagccgcgtgcgcccgggccagcgcgtgtacgtggtggccgagcgtgacggggaccgccggctcctgcccgccgctgtgcacagcgtgaccctaagcgaggaggccgcgggcgcctacgcgccgctcacggcccagggcaccattctcatcaaccgggtgctggcctcgtgctacgcggtcatcgaggagcacagctgggcgcaccgggccttcgcgcccttccgcctggcgcacgcgctcctggctgcactggcgcccgcgcgcacggaccgcggcggggacagcggcggcggggaccgcgggggcggcggcggcagagtagccctaaccgctccaggtgctgccgacgctccgggtgcgggggccaccgcgggcatccactggtactcgcagctgctctaccaaataggcacctggctcctggacagcgaggccctgcacccgctgggcatggcggtcaagtccagcnnnagccggggggccgggggaggggcgcgggagggggcc

SEQ ID NO: 7 - DNA, 1622 bp, Homo sapiens
catcagcccaccaggagacctcgcccgccgctcccccgggctccccggccatgtctcccgcccggctccggccccgactgcacttctgcctggtcctgttgctgctgctggtggtgcccgcggcatggggctgcgggccgggtcgggtggtgggcagccgccggcgaccgccacgcaaactcgtgccgctcgcctacaagcagttcagccccaatgtgcccgagaagaccctgggcgccagcggacgctatgaaggcaagatcgctcgcagctccgagcgcttcaaggagctcacccccaattacaatccagacatcatcttcaaggacgaggagaacacaggcgccgaccgcctcatgacccagcgctgcaaggaccgcctgaactcgctggctatctcggtgatgaaccagtggcccggtgtgaagctgcgggtgaccgagggctgggacgaggacggccaccactcagaggagtccctgcattatgagggccgcgcggtggacatcaccacatcagaccgcgaccgcaataagtatggactgctggcgcgcttggcagtggaggccggctttgactgggtgtattacgagtcaaaggcccacgtgcattgctccgtcaagtccgagcactcggccgcagccaagacgggcggctgcttccctgccggagcccaggtacgcctggagagtggggcgcgtgtggccttgtcagccgtgaggccgggagaccgtgtgctggccatgggggaggatgggagccccaccttcagcgatgtgctcattttcctggaccgcgagccccacaggctgagagccttccaggtcatcgagactcaggaccccccacgccgcctggcactcacacccgctcacctgctctttacggctgacaatcacacggagccggcagcccgcttccgggccacatttgccagccacgtgcagcctggccagtacgtgctggtggctggggtgccaggcctgcagcctgcccgcgtggcagctgtctctacacacgtggccctcggggcctacgccccgctcacaaagcatgggacactggtggtggaggatgtggtggcatcctgcttcgcggccgtggctgaccaccacctggctcagttggccttctggcccctgagactctttcacagcttggcatggggcagctggaccccgggggagggtgtgcattggtacccccagctgctctaccgcctggggcgtctcctgctagaagagggcagcttccacccactgggcatgtccggggcagggagctgaaaggactccaccgctgccctcctggaactgctgtactgggtccagaagcctctcagccaggagggagctggccctggaagggacctgagctgggggacactggctcctgccatctcctctgccatgaagatacaccattgagacttgactgggcaacaccagcgtcccccacccgcgtcgtggtgtagtcatagagctgcaagctgagctggcgaggggatggttgttgacccctctctcctagagaccttgaggctggcacggcgactcccaactcagcctgctctcactacgagttttcatactctgcctcccccattgggagggcccattccc

SEQ ID NO: 8 - DNA, 1191 bp, Homo sapiens
atggctctcctgaccaatctactgcccttgtgctgcttggcacttctggcgctgccagcccagagctgcgggccgggccgggggccggttggccggcgccgctatgcgcgcaagcagctcgtgccgctactctacaagcaatttgtgcccggcgtgccagagcggaccctgggcgccagtgggccagcggaggggagggtggcaaggggctccgagcgcttccgggacctcgtgcccaactacaaccccgacatcatcttcaaggatgaggagaacagtggagccgaccgcctgatgaccgagcgttgcaaggagagggtgaacgctttggccattgccgtgatgaacatgtggcccggagtgcgcctacgagtgactgagggctgggacgaggacggccaccacgctcaggattcactccactacgaaggccgtgctttggacatcactacgtctgaccgcgaccgcaacaagtatgggttgctggcgcgcctcgcagtggaagccggcttcgactgggtctactacgagtcccgcaaccacgtccacgtgtcggtcaaagctgataactcactggcggtccgggcgggcggctgctttccgggaaatgcaactgtgcgcctgtggagcggcgagcggaaagggctgcgggaactgcaccgcggagactgggttttggcggccgatgcgtcaggccgggtggtgcccacgccggtgctgctcttcctggaccgggacttgcagcgccgggcttcatttgtggctgtggagaccgagtggcctccacgcaaactgttgctcacgccctggcacctggtgtttgccgctcgagggccggcgcccgcgccaggcgactttgcaccggtgttcgcgcgccggctacgcgctggggactcggtgctggcgcccggcggggatgcgcttcggccagcgcgcgtggcccgtgtggcgcgggaggaagccgtgggcgtgttcgcgccgctcaccgcgcacgggacgctgctggtgaacgatgtcctggcctcttgctacgcggttctggagagtcaccagtgggcgcaccgcgcttttgcccccttgagactgctgcacgcgctaggggcgctgctccccggcggggccgtccagccgactggcatgcattggtactctcggctcctctaccgcttagcggaggagctactgggctga

SEQ ID NO: 9 - DNA, 1251 bp, Brachydanio rerio
atggacgtaaggctgcatctgaagcaatttgctttactgtgttttatcagcttgcttctgacgccttgtggattagcctgtggtcctggtagaggttatggaaaacgaagacacccaaagaaattaaccccgttggcttacaagcaattcatccccaacgttgctgagaaaacgcttggagccagcggcaaatacgaaggcaaaatcacaaggaattcagagagatttaaagagctgattccgaattataatcccgatatcatctttaaggacgaggaaaacacaaacgctgacaggctgatgaccaagcgctgtaaggacaagttaaattcgttggccatatccgtcatgaaccactggcccggcgtgaaactgcgcgtcactgaaggctgggatgaggatggtcaccatttagaagaatctttgcactatgagggacgggcagtggacatcactacctcagacagggataaaagcaagtatgggatgctatccaggcttgcagtggaggcaggattcgactgggtctattatgaatctaaagcccacatacactgctctgtcaaagcagaaaattcagtggctgctaaatcaggaggatgttttcctgggtctgggacggtgacacttggtgatgggacgaggaaacccatcaaagatcttaaagtgggcgaccgggttttggctgcagacgagaagggaaatgtcttaataagcgactttattatgtttatagaccacgatccgacaacgagaaggcaattcatcgtcatcgagacgtcagaacctttcaccaagctcaccctcactgccgcgcacctagttttcgttggaaactcttcagcagcttcgggtataacagcaacatttgccagcaacgtgaagcctggagatacagttttagtgtgggaagacacatgcgagagcctcaagagcgttacagtgaaaaggatttacactgaggagcacgagggctcttttgcgccagtcaccgcgcacggaaccataatagtggatcaggtgttggcatcgtgctacgcggtcattgagaaccacaaatgggcacattgggcttttgcgccggtcaggttgtgtcacaagctgatgacgtggctttttccggctcgtgaatcaaacgtcaattttcaggaggatggtatccactggtactcaaatatgctgtttcacatcggctcttggctgctggacagagactctttccatccactcgggattttacacttaagttga

SEQ ID NO: 10 - protein, 425 aa, Gallus gallus
MVEMLLLTRILLVGFICALLVSSGLTCGPGRGIGKRRHPKKLTPLAYKQFIPNVAEKTLGASGRYEGKITRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEHGGTKLVKDLSPGDRVLAADADGRLLYSDFLTFLDRMDSSRKLFYVIETRQPRARLLLTAAHLLFVAPQHNQSEATGSTSGQALFASNVKPGQRVYVLGEGGQQLLPASVHSVSLREEASGAYAPLTAQGTILINRVLASCYAVIEEHSWAHWAFAPFRLAQGLLAALCPDGAIPTAATTTTGIHWYSRLLYRIGSWVLDGDALHPLGMVAPAS

SEQ ID NO: 11 - protein, 396 aa, Mus musculus [sequence omitted]

SEQ ID NO: 12 - protein, 411 aa, Mus musculus
MSPAWLRPRLRFCLFLLLLLLVPAARGCGPGRVVGSRRRPPRKLVPLAYKQFSPNVPEKTLGASGRYEGKIARSSERFKELTPNYNPDIIFKDEENTGADRLMTQRCKDRLNSLAISVMNQWPGVKLRVTEGRDEDGHHSEESLHYEGRAVDITTSDRDRNKYGLLARLAVEAGFDWVYYESKAHVHCSVKSEHSAAAKTGGCFPAGAQVRLENGERVALSAVKPGDRVLAMGEDGTPTFSDVLIFLDREPNRLRAFQVIETQDPPRRLALTPAHLLFIADNHTEPAAHFRATFASHVQPGQYVLVSGVPGLQPARVAAVSTHVALGSYAPLTRHGTLVVEDVVASCFAAVADHHLAQLAFWPLRLFPSLAWGSWTPSEGVHSYPQMLYRLGRLLLEESTFHPLGMSGAGS

SEQ ID NO: 13 - protein, 437 aa, Mus musculus
MLLLLARCFLVILASSLLVCPGLACGPGRGFGKRRHPKKLTPLAYKQFIPNVAEKTLGASGRYEGKITRNSERFKELTPNYNPDIIFKDEENTGADRLMTQRCKDKLNALAISVMNQWPGVRLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLRPGDRVLAADDQGRLLYSDFLTFLDRDEGAKKVFYVIETLEPRERLLLTAAHLLFVAPHNDSGPTPGPSALFASRVRPGQRVYVVAERGGDRRLLPAAVHSVTLREEEAGAYAPLTAHGTILINRVLASCYAVIEEHSWAHRAFAPFRLAHALLAALAPARTDGGGGGSIPAAQSATEARGAEPTAGIHWYSQLLYHIGTWLLDSETMHPLGMAVKSS

SEQ ID NO: 14 - protein, 418 aa, Brachydanio rerio
MRLLTRVLLVSLLTLSLVVSGLACGPGRGYGRRRHPKKLTPLAYKQFIPNVAEKTLGASGRYEGKITRNSERFKELTPNYNPDIIFKDEENTGADRLMTQRCKDKLNSLAISVMNHWPGVKLRVTEGWDEDGHHFEESLHYEGRAVDITTSDRDKSKYGTLSRLAVEAGFDWVYYESKAHIHCSVKAENSVAAKSGGCFPGSALVSLQDGGQKAVKDLNPGDKVLAADSAGNLVFSDFIMFTDRDSTTRRVFYVIETQEPVEKITLTAAHLLFVLDNSTEDLHTMTAAYASSVRAGQKVMVVDDSGQLKSVIVQRIYTEEQRGSFAPVTAHGTIVVDRILASCYAVIEDQGLAHLAFAPARLYYYVSSFLSPKTPAVGPMRLYNRRGSTGTPGSCHQMGTWLLDSNMLHPLGMSVNSS

SEQ ID NO: 15 - protein, 475 aa, Homo sapiens
  SITE (463): Xaa=unknown amino acid residue
MLLLARCLLLVLVSSLLVCSGLACGPGRGFGKRRHPKKLTPLAYKQFIPNVAEKTLGASGRYEGKISRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLSPGDRVLAADDQGRLLYSDFLTFLDRDDGAKKVFYVIETREPRERLLLTAAHLLFVAPHNDSATGEPEASSGSGPPSGGALGPRALFASRVRPGQRVYVVAERDGDRRLLPAAVHSVTLSEEAAGAYAPLTAQGTILINRVLASCYAVIEEHSWAHRAFAPFRLAHALLAALAPARTDRGGDSGGGDRGGGGGRVALTAPGAADAPGAGATAGIHWYSQLLYQIGTWLLDSEALHPLGMAVKSSXSRGAGGGAREGA

SEQ ID NO: 16 - protein, 411 aa, Homo sapiens
MSPARLRPRLHFCLVLLLLLVVPAAWGCGPGRVVGSRRRPPRKLVPLAYKQFSPNVPEKTLGASGRYEGKIARSSERFKELTPNYNPDIIFKDEENTGADRLMTQRCKDRLNSLAISVMNQWPGVKLRVTEGWDEDGHHSEESLHYEGRAVDITTSDRDRNKYGLLARLAVEAGFDWVYYESKAHVHCSVKSEHSAAAKTGGCFPAGAQVRLESGARVALSAVRPGDRVLAMGEDGSPTFSDVLIFLDREPHRLRAFQVIETQDPPRRLALTPAHLLFTADNHTEPAARFRATFASHVQPGQYVLVAGVPGLQPARVAAVSTHVALGAYAPLTKHGTLVVEDVVASCFAAVADHHLAQLAFWPLRLFHSLAWGSWTPGEGVHWYPQLLYRLGRLLLEEGSFHPLGMSGAGS

SEQ ID NO: 17 - protein, 396 aa, Homo sapiens
MALLTNLLPLCCLALLALPAQSCGPGRGPVGRRRYARKQLVPLLYKQFVPGVPERTLGASGPAEGRVARGSERFRDLVPNYNPDIIFKDEENSGADRLMTERCKERVNALAIAVMNMWPGVRLRVTEGWDEDGHHAQDSLHYEGRALDITTSDRDRNKYGLLARLAVEAGFDWVYYESRNHVHVSVKADNSLAVRAGGCFPGNATVRLWSGERKGLRELHRGDWVLAADASGRVVPTPVLLFLDRDLQRRASFVAVETEWPPRKLLLTPWHLVFAARGPAPAPGDFAPVFARRLRAGDSVLAPGGDALRPARVARVAREEAVGVFAPLTAHGTLLVNDVLASCYAVLESHQWAHRAFAPLRLLHALGALLPGGAVQPTGMHWYSRLLYRLAEELLG

SEQ ID NO: 18 - protein, 416 aa, Brachydanio rerio
MDVRLHLKQFALLCFISLLLTPCGLACGPGRGYGKRRHPKKLTPLAYKQFIPNVAEKTLGASGKYEGKITRNSERFKELIPNYNPDIIFKDEENTNADRLMTKRCKDKLNSLAISVMNHWPGVKLRVTEGWDEDGHHLEESLHYEGRAVDITTSDRDKSKYGMLSRLAVEAGFDWVYYESKAHIHCSVKAENSVAAKSGGCFPGSGTVTLGDGTRKPIKDLKVGDRVLAADEKGNVLISDFIMFIDHDPTTRRQFIVIETSEPFTKLTLTAAHLVFVGNSSAASGITATFASNVKPGDTVLVWEDTCESLKSVTVKRIYTEEHEGSFAPVTAHGTIIVDQVLASCYAVIENHKWAHWAFAPVRLCHKLMTWLFPARESNVNFQEDGIHWYSNMLFHIGSWLLDRDSFHPLGILHLS

SEQ ID NO: 19 - DNA, 1416 bp, Drosophila melanogaster
  CDS (1)..(1413)
atggataaccacagctcagtgccttgggccagtgccgccagtgtcacctgtctctccctgggatgccaaatgccacagttccagttccagttccagctccaaatccgcagcgagctccatctccgcaagcccgcaagaagaacgcaaacgatgcgccacattgcgcatacgcagcgttgcctcagcaggctgacctctctggtggccctgctgctgatcgtcttgccgatggtctttagcccggctcacagctgcggtcctggccgaggattgggtcgtcatagggcgcgcaacctgtatccgctggtcctcaagcagacaattcccaatctatccgagtacacgaacagcgcctccggacctctggagggtgtgatccgtcgggattcgcccaaattcaaggacctcgtgcccaactacaacagggacatccttttccgtgacgaggaaggcaccggagcggatggcttgatgagcaagcgctgcaaggagaagctaaacgtgctggcctactcggtgatgaacgaatggcccggcatccggctgctggtcaccgagagctgggacgaggactaccatcacggccaggagtcgctccactacgagggccgagcggtgaccattgccacctccgatcgcgaccagtccaaatacggcatgctcgctcgcctggccgtcgaggctggattcgattgggtctcctacgtcagcaggcgccacatctactgctccgtcaagtcagattcgtcgatcagttcccacgtgcacggctgcttcacgccggagagcacagcgctgctggagagtggagtccggaagccgctcggcgagctctctatcggagatcgtgttttgagcatgaccgccaacggacaggccgtctacagcgaagtgatcctcttcatggaccgcaacctcgagcagatgcaaaactttgtgcagctgcacacggacggtggagcagtgctcacggtgacgccggctcacctggttagcgtttggcagccggagagccagaagctcacgtttgtgtttgcgcatcgcatcgaggagaagaaccaggtgctcgtacgggatgtggagacgggcgagctgaggccccagcgagtggtcaagttgggcagtgtgcgcagtaagggcgtggtcgcgccgctgacccgcgagggcaccattgtggtcaactcggtggccgccagttgctatgcggtgatcaacagtcagtcgctggcccactggggactggctcccatgcgcctgctgtccacgctggaggcgtggctgcccgccaaggagcagttgcacagttcgccgaaggtggtgagctcggcgcagcagcagaatggcatccattggtatgccaatgcgctctacaaggtcaaggactacgtgctgccgcagagctggcgccacgattga

SEQ ID NO: 20 - protein, 471 aa, Drosophila melanogaster
MDNHSSVPWASAASVTCLSLGCQMPQFQFQFQLQIRSELHLRKPARRTQTMRHIAHTQRCLSRLTSLVALLLIVLPMVFSPAHSCGPGRGLGRHRARNLYPLVLKQTIPNLSEYTNSASGPLEGVIRRDSPKFKDLVPNYNRDILFRDEEGTGADGLMSKRCKEKLNVLAYSVMNEWPGIRLLVTESWDEDYHHGQESLHYEGRAVTIATSDRDQSKYGMLARLAVEAGFDWVSYVSRRHIYCSVKSDSSISSHVHGCFTPESTALLESGVRKPLGELSIGDRVLSMTANGQAVYSEVILFMDRNLEQMQNFVQLHTDGGAVLTVTPAHLVSVWQPESQKLTFVFAHRIEEKNQVLVRDVETGELRPQRVVKLGSVRSKGVVAPLTREGTIVVNSVAASCYAVINSQSLAHWGLAPMRLLSTLEAWLPAKEQLHSSPKVVSSAQQQNGIHWYANALYKVKDYVLPQSWRHD

SEQ ID NO: 21 - protein, 221 aa, Artificial Sequence
  Description of Artificial Sequence Degenerate Shh polypeptide general formula
CGPGRGXGXRRHPKKLTPLAYKQFIPNVAEKTLGASGRYEGKIXRNSERFKELTPNYNPDIIFKDEENTGADRLMTQRCKDKLNXLAISVMNXWPGVXLRVTEGWDEDGHHXEESLHYEGRAVDITTSDRDXSKYGXLXRLAVEAGFDWVYYESKAHIHCSVKAENSVAAKSGGCFPGSAXVXLXXGGXKXVKDLXPGDXVLAADXXGXLXXSDFXXFXDR

SEQ ID NO: 22 - protein, 167 aa, Artificial Sequence
  Description of Artificial Sequence Degenerate hedgehog polypeptide general formula
CGPGRGXXXRRXXXPKXLXPLXYKQFXPXXXEXTLGASGXXEGXXXRXSERFXXLTPNYNPDIIFKDEENXGADRLMTXRCKXXXNXLAISVMNXWPGVXLRVTEGXDEDGHHXXXSLHYEGRAXDITTSDRDXXKYGXLXRLAVEAGFDWVYYESXXHXHXSVKXX

SEQ ID NO: 23 - DNA, 74 bp, Artificial Sequence
  primer
gcgcgcttcgaagcgaggcagccagcgagggagagagcgagcgggcgagccggagcgaggaaatcgatgcgcgc

SEQ ID NO: 24 - DNA, 74 bp, Artificial Sequence
  primer
gcgcgcagatctgggaaagcgcaagagagagcgcacacgcacacacccgccgcgcgcactcgggatccgcgcgc

SEQ ID NO: 25 - DNA, 997 bp, Artificial Sequence
  gene activation construct
cgaagcgaggcagccagcgagggagagagcgagcgggcgagccggagcgaggaaatcgaaggttcgaatccttcccccaccaccatcactttcaaaagtccgaaagaatctgctccctgcttgtgtgttggaggtcgctgagtagtgcgcgagtaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattggacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatcgatctgggaaagcgcaagagagagcgcacacgcacacacccgccgcgcgcactcgg

SEQ ID NO: 26 - DNA, 26 bp, Artificial Sequence
  antisense construct
gtcctggcgccgccgccgccgtcgcc

SEQ ID NO: 27 - DNA, 26 bp, Artificial Sequence
  antisense construct
ttccgatgaccggcctttcgcggtga

SEQ ID NO: 28 - DNA, 26 bp, Artificial Sequence
  antisense construct
gtgcacggaaaggtgcaggccacact

SEQ ID NO: 29 - DNA, 19 bp, Artificial Sequence
  primer
aatggaagaccactcccac

SEQ ID NO: 30 - DNA, 18 bp, Artificial Sequence
  primer
tggagatgcaggctccac

SEQ ID NO: 31 - DNA, 22 bp, Artificial sequence
  primer
acaccttctacaatgagctgcg

SEQ ID NO: 32 - DNA, 21 bp, Artificial Sequence
  primer
cgctcggtgaggatcttcatg

SEQ ID NO: 33 - DNA, 20 bp, Artificial Sequence
  primer
atgtgtgatgcttttgtagg

SEQ ID NO: 34 - DNA, 19 bp, Artificial Sequence
  primer
ttttccatcccatttctgc

SEQ ID NO: 35 - DNA, 20 bp, Artificial Sequence
  primer
ctgatactctgggatatggg

SEQ ID NO: 36 - DNA, 20 bp, Artificial Sequence
  primer
tcaagtcgaggacactggct

SEQ ID NO: 37 - DNA, 23 bp, Artificial Sequence
  primer
gcattggcaggaggagttgattg

SEQ ID NO: 38 - DNA, 23 bp, Artificial Sequence
  primer
gggtcgtggttgtgaagggaagc

(19) United States
(12) Patent Application Publication
(10) Pub. No.: US 2004/0171533 A1
(43) Pub. Date: Sep. 2, 2004

(54) Methods and compositions for regulating adiopocytes

(76) Inventors: Barbara Zehentner, Viecht (DE); Ulrike Leser-Reiff, Penzberg (DE); Helmut Burtscher, Habach (DE)

Correspondence Address: ROPES & GRAY LLP, ONE INTERNATIONAL PLACE, BOSTON, MA, 02110-2624, US

(21) Appl. No.: 09/795,917

(22) Filed: Feb. 28, 2001

Related U.S. Application Data
(60) Provisional application No. 60/186,058, filed on Feb. 29, 2000.

(51) Int. Cl. A61K 38/17; C12N 15/85

(52) U.S. Cl. 514/12; 435/455

(57) ABSTRACT

The present application relates to a method for modulating the formation and/or maintenance of adipocyte tissue by ectopically contacting adipocyte cells, especially adipocyte stem/progenitor cells, in vitro or in vivo, with a hedgehog therapeutic or ptc therapeutic in an amount effective to alter the growth state the treated cells, e.g., relative to the absence of administeration of the hedgehog therapeutic or ptc therapeutic.

### REFERENCE TO RELATED APPLICATIONS

This application claims priority to U.S. Provisional Application No. 60/186,058, filed Feb. 29, 2000, the specification of which are herein incorporated by reference.

### BACKGROUND OF THE INVENTION

Adipocytes are highly specialized cells that play a critical role in energy and homeostasis. Their primary role is to store triglycerides in times of caloric excess and to mobilize this reserver during periods of nutritional deprivation. Adipocytes are derived from a multipotent stem cell of mesodermal origin that also gives rise to the adipocyte and cartilage lineages. Adipocyte differentiation is characterized by a coordinate increase in adipocyte-specific gene expression.

Recent years have seen important advances in our understanding of the molecular basis of adipocyte differentiation. (reviewed in Cornelius, P. et al. (1994) Annu. Rev. Nutr. 14:99-129; Tontonoz, P. et al. (1995) Curr. Opin. Genet. Dev. 5:571-576. A number of transcription factors are induced in fat cell differentiation (C/EBPα, C/EBPβ and ADD1/SREBP1) and influence this process to a certain extent (Freytag, S. O. et al. (1994) Genes Dev. 8:1654-63; Kim, J. B. and Spiegelman, B. M. (1996) Genes Dev. 10:1096-1107; Lin, F. T. and Lane, M. D. (1994) PNAS USA 91:8757-61; Samuelsson, L. et al. (1991) EMBO J. 10:3787-93; Tontonoz, P. et al. (1993) Mol Cell Biol 13:4753-9; Umek, R. M. et al. (1991) Science 251:288-92; Wu, C. L. et al. (1995) Mol Cell Biol 15:253646; Yeh, W. C. et al. (1995) Genes Dev. 9:168-81).

The peroxisome proliferator-activated receptors, or “PPAR”, are members of the type II class of steroid/thyroid superfamily of receptors and which mediate the pleiotropic effects of peroxisome proliferators. Type II class of nuclear receptors includes PPAR, the thyroid hormone receptor (T₃R), and the vitamin D₃ receptor (VD₃R). Type II receptors are functionally distinct from the classical steroid receptors, such as the glucocorticoid receptor, the progesterone receptor and the estrogen receptor (reviewed in Stunnenberg, H. G. (1993) BioEssays Vol. 15 (5): 309-15. Three properties distinguish these two classes. Firstly, type II receptors are able to bind to their responsive elements in the absence of ligand (Damm et al. (1989) Nature 339:593-597; Sap et al., Nature 340:242-244; De The et al. (1990) Nature 343:177-180), whereas ligand binding is required to dissociate to the type I receptor-hsp 90 complex and hence indirectly governs DNA binding. Secondly, type II receptors bind and transactivate through responsive elements that are composed of half-sites arranged as direct repeats, as opposed to palindromically arranged half-sites invariably separated by three nucleotides required by type I receptors. Finally, type II receptors do not bind to their respective binding site as homodimers but require an auxiliary factor, RXR (e.g., RXR , RXR , RXR ) for high affinity binding (Yu et al. (1991) Cell 67:1251-1266; Bugge et al. (1992) EMBO J. 11:1409-1418; Kliewer et al. (1992) Nature 355:446-449; Leid et al. (1992) Cell 68:377-395; Marks et al. (1992) EMBO J. 11:1419-1435; Zhang et al. (1992) Nature 355:441-446). The interaction between type II receptors requires a region in the C-terminal domain (Yu et al. (1991) Cell 67:1251-1266; Kliewer et al. (1992) Nature 355:446-449; Leid et al. (1992) Cell 68:377-395; Marks et al. (1992) EMBO J. 11:1419-1435). Following binding, the transcriptional activity of a target gene (i.e., a gene associated with the specific DNA sequence) is enhanced as a function of the ligand bound to the receptor heterodimer.

### SUMMARY OF THE INVENTION

One aspect of the present application relates to a method for regulating the formation and/or maintenance of adipocyte tissue by ectopically contacting adipocyte cells, especially adipocyte stem/progenitor cells, in vitro or in vivo, with a hedgehog therapeutic or ptc therapeutic in an amount effective to alter the growth state the treated cells, e.g., relative to the absence of administeration of the hedgehog therapeutic or ptc therapeutic.

Wherein the subject method is carried out using a hedgehog therapeutic, the hedgehog therapeutic preferably a polypeptide including a hedgehog portion comprising at least a bioactive extracellular portion of a hedgehog protein, e.g., the hedgehog portion includes at least 50, 100 or 150 (contiguous) amino acid residues of an N-terminal half of a hedgehog protein. In preferred embodiments, the hedgehog portion includes at least a portion of the hedgehog protein corresponding to a 19 kd fragment of the extracellular domain of a hedgehog protein.

In preferred embodiments, the hedgehog portion has an amino acid sequence at least 60, 75, 85, or 95 percent identical with a hedgehog protein of any of SEQ ID Nos. 10-18 or 20, though sequences identical to those sequence listing entries are also contemplated as useful in the present method. The hedgehog portion can be encoded by a nucleic acid which hybridizes under stringent conditions to a nucleic acid sequence of any of SEQ ID Nos. 1-9 or 19, e.g., the hedgehog portion can be encoded by a vertebrate hedgehog gene, especially a human hedgehog gene.

In other embodiments, the subject method can be carried out by administering a gene activation construct, wherein the gene activation construct is deigned to recombine with a genomic hedgehog gene of the patient to provide a heterologous transcriptional regulatory sequence operatively linked to a coding sequence of the hedgehog gene.

In still other embodiments, the subject method can be practiced with the administration of a gene therapy construct encoding a hedgehog polypeptide. For instance, the gene therapy construct can be provided in a composition selected from a group consisting of a recombinant viral particle, a liposome, and a poly-cationic nucleic acid binding agent,

In yet other embodiments, the subject method can be carried out using a ptc therapeutic. An exemplary ptc therapeutic is a small organic molecule which binds to a patched protein and derepresses patched-mediated inhibition of mitosis, e.g., a molecule which binds to patched and mimics hedgehog-mediated patched signal transduction, which binds to patched and regulates patched-dependent gene expression. For instance, the binding of the ptc therapeutic to patched may result in upregulation of patched and/or gli expression.

In a more generic sense, the ptc therapeutic can be a small organic molecule which interacts with adipocyte cells to induce hedgehog-mediated patched signal transduction, such as by altering the localization, protein-protein binding and/or enzymatic activity of an intracellular protein involved in a patched signal pathway. For instance, the ptc therapeutic may alter the level of expression of a hedgehog protein, a patched protein or a protein involved in the intracellular signal transduction pathway of patched.

In certain embodiments, the ptc therapeutic is an antisense construct which inhibits the expression of a protein which is involved in the signal transduction pathway of patched and the expression of which antagonizes hedgehog-mediated signals. The antisense construct is perferably an oligonucleotide of about 20-30 nucleotides in length and having a GC content of at least 50 percent.

In other embodiments, the ptc therapeutic is an inhibitor of protein kinase A (PKA), such as a 5-isoquinolinesulfonamide. The PKA inhibitor can be a cyclic AMP analog. Exemplary PKA inhibitors include N-[2-((p-bromocinnamyl)amino)ethyl]-5-isoquinolinesulfonamide, 1-(5-isoquinoline-sulfonyl)-2-methylpiperazine, KT5720, 8-bromo-cAMP, dibutyryl-cAMP and PKA Heat Stable Inhibitor isoform. Another exemplary PKA inhibitor is represented in the general formula:

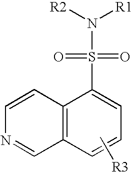

wherein,

R₁ and R₂ each can independently represent hydrogen, and as valence and stability permit a lower alkyl, a lower alkenyl, a lower alkynyl, a carbonyl (such as a carboxyl, an ester, a formate, or a ketone), a thiocarbonyl (such as a thioester, a thioacetate, or a thioformate), an amino, an acylamino, an amido, a cyano, a nitro, an azido, a sulfate, a sulfonate, a sulfonamido, —(CH₂)ₘ—R₈, —(CH₂)ₘ—OH, —(CH₂)ₘ—O-lower alkyl, —(CH₂)ₘ—O-lower alkenyl, —(CH₂)ₙ—O—(CH₂)ₘ—R₈, —(CH₂)ₘ—SH, —(CH₂)ₘ—S-lower alkyl, —(CH₂)ₘ—S-lower alkenyl, —(CH₂)ₙ—S—(CH₂)ₘ—R₈, or

R₁ and R₂ taken together with N form a heterocycle (substituted or unsubstituted);

R₃ is absent or represents one or more substitutions to the isoquinoline ring such as a lower alkyl, a lower alkenyl, a lower alkynyl, a carbonyl (such as a carboxyl, an ester, a formate, or a ketone), a thiocarbonyl (such as a thioester, a thioacetate, or a thioformate), an amino, an acylamino, an amido, a cyano, a nitro, an azido, a sulfate, a sulfonate, a sulfonamido, —(CH₂)ₘ—R₈, —(CH₂)ₘ—OH, —(CH₂)ₘ—O-lower alkyl, —(CH₂)ₘ—O-lower alkenyl,—(CH₂)ₙ—O—(CH₂)ₘ—R₈, —(CH₂)ₘ—SH, —(CH₂)ₘ—S-lower alkyl, —(CH₂)ₘ—S-lower alkenyl, —(CH₂)ₙ—S—(CH₂)ₘ—R₈;

R₈ represents a substituted or unsubstituted aryl, aralkyl, cycloalkyl, cycloalkenyl, or heterocycle; and

n and m are independently for each occurrence zero or an integer in the range of 1 to 6.

### BRIEF DESCRIPTION OF THE DRAWINGS

FIG. 1. PPAR-γ, aP2, gli and ptc expression in C3H10T1/2 cells. The PCR fragments of the standard vector (b) and the cell mRNA (a) were separated by agarose gels. Two types of experiment were performed: samples 1 are derived from 72 h induction without serum and samples 2 from 11 d with serum. Each experiment consists of a control (0), the BMP-2 treated (1), Shh treated (2), and the combination of Shh and BMP-2 (3) treated sample. The charts show the relative expression levels resulting from three independent measurements, normalized with their actin content, for BMP-2 (▪), Shh ( ), and BMP-2 plus Shh ( ) treated cells. The bold base line at a expression level of one is equivalent to the expression level of the control sample.

FIG. 2. Oil Red O staining of C3H10T1/2 cells. Cells were cultured for 11 d with FCS and then cytologically stained with Oil Red O (see Material and Methods). Lipid filled vesicles appear red in the cytosol of adipocyte-like cells. Pictures with 10-fold magnification show the untreated cells as a control (A), cells treated with BMP-2 (B), cells treated with Shh (C), and cells treated with Shh and BMP-2 simultaneously (D).

### DETAILED DESCRIPTION OF THE INVENTION

### I. Overview

Skeletal tissue is composed of various types of mesenchymal cells, like osteoblasts, chondrocytes, and adipocytes. These cells originate from common pluripotent progenitors, known as mesenchymal stem cells (Bruder et al., 1994). A cell system with comparable multipotentiality is the mouse embryonic fibroblastic cell line C3H10T1/2, capable of in vitro myogenesis, osteogenesis, chondrogenesis and adipogenesis. Bone morphogenic protein-2 (BMP-2) is an important signaling protein that influences maturation of mesenchymal cells. It has been shown to cause differentiation of C3H10T1/2 cells into adipocytes, chondrocytes and osteoblasts (Ahrens et al., 1993, Wang et al., 1993). Bone morphogenic proteins, originally isolated from bone, are part of the transforming growth factor-β (TGF-β) superfamily (Kawabata et al., 1998) and consist of at least 15 molecules. Several BMPs are able to induce ectopic bone formation (Wang et al., 1988).

During embryonic development BMP-2 is secreted in the mesoderm and apical ectodermal ridge of the mouse limb in response to Sonic Hedgehog (Shh) (Laufer et al., 1994) pointing to BMP-2 as a downstream target of Shh. Recent results indicate that Shh is able to induce ectopic bone formation in a similar manner as BMP-2 (Kinto et al., 1997). BMP-2 and Shh are able to stimulate alkaline phosphatase activity (Katagiri et al., 1990, Nakamura et al, 1997), a marker indicating osteogenesis, in C3H10T1/2 cells.

Since parallels between Shh and BMP-2 have only been reported for osteogenic differentiation, our aim was to investigate if Shh also influences adipogenesis like BMP-2. We determined the adipocyte-like phenotype of C3H10T1/2 cells by staining lipid vesicles in the cytosol with Oil Red O. Quantitative RT-PCR was used to monitor the expression of adipocytic marker genes, PPAR-γ and aP2, and of the hedgehog responsive genes, gli and patched. Gli and Patched (Ptc) are important components of the hedgehog signaling pathway. Gli is the first of three identified vertebrate homologs (Gli,Gli2,Gli3) of the Drosophila zincfinger transcription factor cubitus interruptus (Ci). Gli, like Ci, is involved in the hedgehog signaling pathway and activates ptc transcription (Platt et al, 1997). Patched is a hedgehog receptor and by itself a transcriptional target of hedgehog (Marigo et al., 1996). Peroxisome proliferator activated receptor γ, PPAR-γ, is a steroid hormone receptor expressed in adipose tissue, activated by fatty acids. PPAR-γ is sufficient to activate the adipocyte-specific enhancer in nonadipocyte cell lines (Elbrecht et al., 1996, Tontonoz et al., 1994). Adipocyte protein 2 (aP2) is an intracellular lipid carrier protein and its expression indicates late stages of the adipocytic differentiation (Matarese and Bernlohr, 1988).

By measuring the expression level of these four marker genes the molecular status of the cells can be determined regarding adipocytic differentiation and stimulation of the hedgehog signaling cascade. In this study we demonstrate that BMP-2 and Shh have contrary effects regarding adipogenesis and that Shh can even counteract BMP-2 stimulation.

As described in more detail below, we monitored adipocytic differentiation in C3H10T1/2 cells by expression studies and cytological staining and demonstrated that Sonic Hedgehog inhibits the adipogenic potential of BMP-2. BMP-2 stimulated the upregulation of two adipocyte markers, PPAR-γ and aP2, in C3H10T1/2 cells measured as early as 72 h under serumfree conditions and as late as 11 d in the presence of 10% FCS. An induction period of 11 d was necessary for detection of a significant percentage of adipocyte-like cells by Oil Red O staining. In contrast to BMP-2, Sonic hedgehog, a postulated downstream target, inhibited adipocyte-like differentiation. Shh by itself and in combination with BMP-2 did neither upregulate the expression of adipocytic marker genes nor induce the maturation into adipose cells filled with lipid vesicles stainable by Oil Red O.

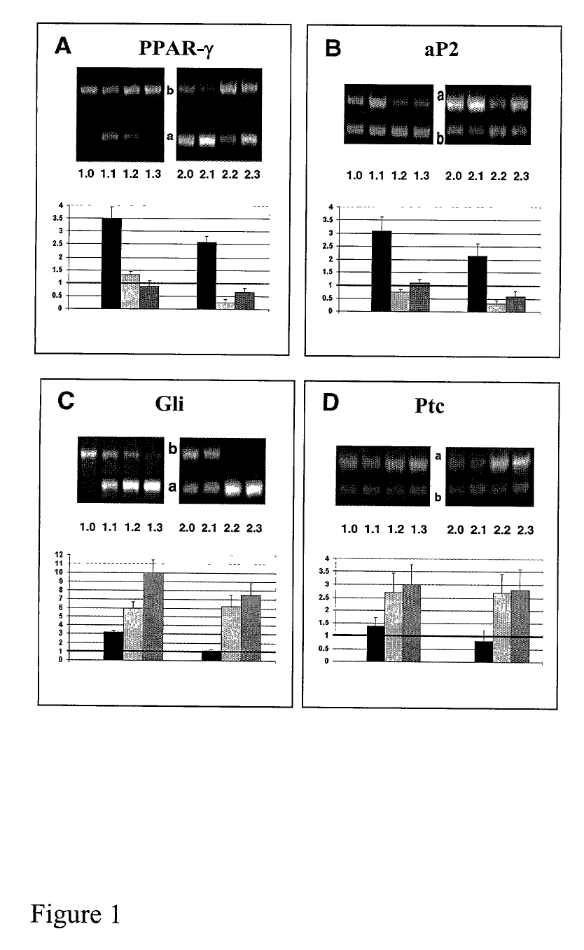

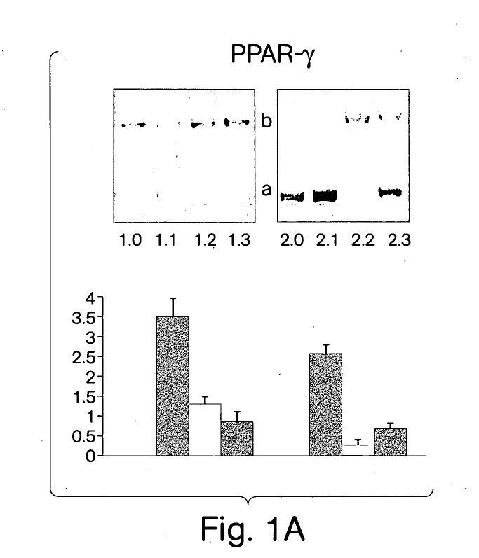

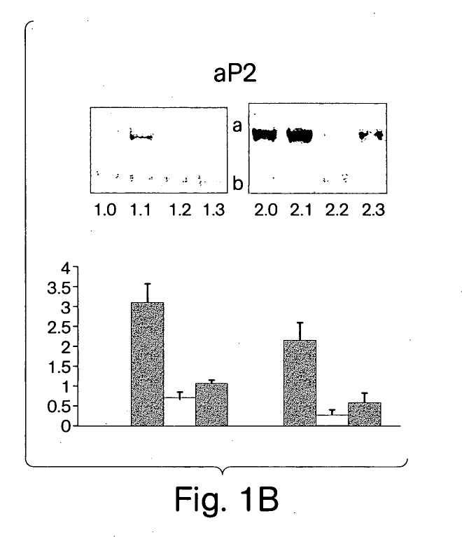

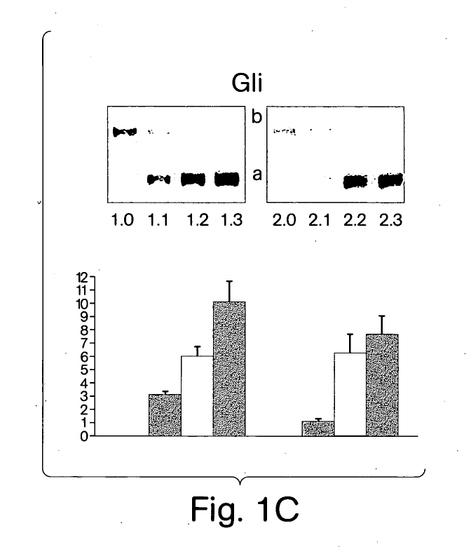

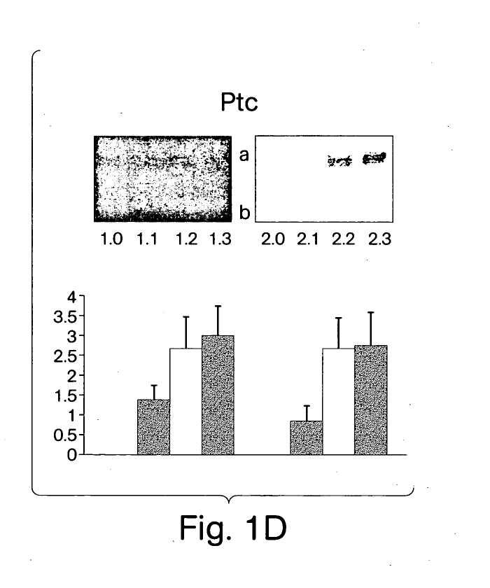

Since serum has been reported to influence adipogenesis (Diascro et al., 1998) serum and a long culture might cause adipogenesis in our cell system. However, even after only 72 h of serumfree culture BMP-2 was able to stimulate the expression of the two adipocytic marker genes PPAR-γ and aP2. Since no fat-like cells could be detected by Oil Red O staining at this time point, an explanation could be that it takes much longer for the appearance of a mature adipocyte-like cell phenotype than it takes for adipocyte specific messages to be upregulated. In order to cultivate C3H10T1/2 cells for longer than 72 h, serum supplement is mandatory and therefore a long term experiment without FCS was not feasible. The expression of adipocytic markers was slightly stimulated by serum (FIG. 1A and B, samples 2.0 compared to samples 1.0). But there has been no detection of fat cells by Oil Red O staining, whereas BMP-2 treatment caused the appearance of a significant amount of mature adipocytes.

Recent data of our lab showed that C3H10T1/2 cells treated with BMP-2 express a chondrocytic expression profile after 24 h, whereas the osteoblastic marker osteocalcin is induced after 72 h (Zehentner et al., 1999). It can be concluded, that adipocytic genes are switched on by BMP-2 simultanously with chondro- and osteoblastic markers during the first three days of treatment. Treatment for a longer period of time then leads to maturation into adipocyte like cells.

Besides monitoring the effect of BMP-2 on adipogenesis, we focused on the influence of Sonic hedgehog protein. The expression of the hedgehog downstream targets gli and ptc was upregulated by Shh at each time tested pointing to functional Shh signaling in our cell system. The combination of Shh and BMP-2 also caused ptc and gli mRNA upregulation, but interestingly gli expression was increased synergistically. BMP-2 by itself already upregulated gli expression 3-fold in the 72 h experiment. In combination with Shh the relative expression level of gli was stimulated about 6- to 10-fold. A positive feedback loop of BMP-2 to Shh signaling could be the reason for this synergistic effect.

After 11 d of BMP-2 treatment and the acquirement of a mature adipocyte-like cell phenotype, BMP-2 did not influence the expression of the transcription factor gli anymore. In contrast to 72 h the final commitment after 11 d to the adipocytic lineage could be a reason for a different gli response. Hence after suppression of adipocytic maturation by Shh co-treatment, BMP-2 again could cause increase of gli expression after 11 d in comparison to Shh alone (FIG. C, sample 2.3).

We could demonstrate in this study that Shh suppresses the adipocytic phenotype and acts antagonistic to BMP-2. Whereas BMP-2 induces adipogenesis, co-treatment with Sonic hedgehog could inhibit the upregulation of adipocytic gene expression and the maturation into lipid vesicle filled adipose cells. Shh alone did not change the expression of adipocyte marker genes after 72 h of treatment and it even caused their downregulation in the long-term culture with FCS. The reason for this could be that FCS by itself slightly upregulates adipocytic differentiation which is in turn suppressed by Shh.

An explanation for contrary effects could be that Shh and BMP-2 are able to stimulate different transduction pathways besides their common signaling. With the use of specific signaling Shh could be able to keep cells in a more undifferentiated state preventing them from maturing towards fat cells. Closer insights into Shh and BMP-2 signaling have to be gained in order to reveal the origin for different mechanisms how to influence cell differentiation. With our work we made a first step to unravel differences between Shh and BMP-2 signaling by demonstrating their contrary effects regarding adipogenesis.

Certain aspects of the invention are directed to a preparations of hedgehog polypeptides, or other molecules which regulate patched or smoothened signalling, and their uses in regulate adipocyte growth or differentiation in mammals. In particular embodiments, the invention is directed to the use of hedgehog polypeptides, as well as agonoist and antagonists thereof, to regulate adipocyte growth and differentiation.

As described in the appended examples, hedgehog proteins are implicated in the proliferation and/or differentiation of adipocytic cells and may provide early signals that regulate the differentiation of these or other precursor (stem) cells into adiposte tissues. In general, the method of the present invention comprises contacting pre-adipocyte cells (e.g., adipocyte stem cells), and adipocytic or other differentiated adipocyte cells, with an amount of a hedgehog therapeutic (defined infra) which produces a non-toxic response by the cell of either (i) inhibition of of adipocyte tissue formation or maintenance of existing adipocyte tissue, or (ii) indution of adipocyte tissue formation, depending on the whether the hedgehog therapeutic is a sufficient hedgehog agonist or hedgehog antagonist. The subject method can be carried out on adipocyte cells which may be either dispersed in culture or a part of an intact tissue or organ. Moreover, the method can be performed on cells which are provided in culture (in vitro), or on cells in a whole animal (in vivo).

In one aspect, the present invention provides pharmaceutical preparations and methods for controlling the formation of adipocytic-derived tissue utilizing, as an active ingredient, a hedgehog polypeptide or a mimetic thereof. The invention also relates to methods of controlling the functional performance of an adipocyte-derived tissue by use of the pharmaceutical preparations of the invention.

The hedgehog formulations of the present invention may be used as part of regimens in the treatment or prevention of disorders of, or surgical or cosmetic repair of, such adipocyte tissues.

In certain embodiments, the subject compositions can be used to inhibit, rather than promote, growth of adipocytic-derived tissue. For instance, certain of the compositions disclosed herein may be applied to the treatment or prevention of a variety hyperplastic or neoplastic conditions affecting adipocyte tissue. The method can find application for the treatment or prophylaxis of, e.g., soft tissue tumors, especially adipose cell tumors, e.g., lipomas, fibrolipomas, lipoblastomas, lipomatosis, hibernomas, hemangiomas and/or liposarcomas.

The subject hedgehog treatments are effective on both human and animal subjects afflicted with these conditions. Animal subjects to which the invention is applicable extend to both domestic animals and livestock, raised either as pets or for commercial purposes. Examples are dogs, cats, cattle, horses, sheep, hogs and goats.

Still another aspect of the present invention provides a method of regulating the growth and differentiation of adipocyte cells and tissues in culture.

Without wishing to be bound by any particular theory, the effect of native hedgehog proteins on the regulation of adipocyte differentiation may be due at least in part to the ability of these proteins to antagonize (directly or indirectly) patched-mediated regulation of gene expression and other physiological effects mediated by that protein. The patched gene product, a cell surface protein, is understood to signal through a pathway which causes transcriptional repression of members of the Wnt and Dpp/BMP families of morphogens, proteins which impart positional information. In development of the CNS and patterning of limbs in vertebrates, the introduction of hedgehog relieves (derepresses) this inhibition conferred by patched, allowing expression of particular gene programs.

Recently, it has been reported that mutations in the human version of patched, a gene first identified in a fruit fly developmental pathway, cause a hereditary skin cancer and may contribute to sporadic skin cancers. See, for example, Hahn et al. (1996) Cell 86:841-851; and Johnson et al. (1996) Science 272:1668-1671. The demonstraction that nevoid basal-cell carcinoma (NBCC) results from mutations in the human patched gene provided an example of the roles patched plays in post-embryonic deveolpment. These observations have led the art to understand one activity of patched to be a tumor suppressor gene, which may act by inhibiting proliferative signals from hedgehog. Our observations set forth below reveal potential new roles for the hedgehog/patched pathway in maintenance of adipocyte cell proliferation and differentiation. Accordingly, the present invention contemplates the use of other agents which are capable of mimicking the effect of the hedgehog protein on patched signalling, e.g., as may be identified from the drug screening assays described below.

### II. Definitions

For convience, certain terms employed in the specfication, examples, and appended claims are collected here.

The term “hedgehog therapeutic” refers to various forms of hedgehog polypeptides, as well as peptidomimetics, which can modulate the proliferation/differentiation state of adipocyte cells by, as will be clear from the context of individual examples, mimicing or potentiating (agonizing) or inhibiting (antagonizing) the effects of a naturally-occurring hedgehog protein. A hedgehog therapeutic which mimics or potentiates the activity of a wild-type hedgehog protein is a “hedgehog agonist”. Conversely, a hedgehog therapeutic which inhibits the activity of a wild-type hedgehog protein is a “hedgehog antagonist”.

In particular, the term “hedgehog polypeptide” encompasses preparations of hedgehog proteins and peptidyl fragments thereof, both agonist and antagonist forms as the specific context will make clear.

As used herein the term “bioactive fragment of a hedgehog protein” refers to a fragment of a full-length hedgehog polypeptide, wherein the fragment specifically agonizes or antagonizes inductive events mediated by wild-type hedgehog proteins. The hedgehog biactive fragment preferably is a soluble extracellular portion of a hedgehog protein, where solubility is with reference to physiologically compatible solutions. Exemplary bioactive fragments are described in PCT publications WO 95/18856 and WO 96/17924.

The term “ptc therapeutic” refers to agents which either (i) mimic the effect of hedgehog proteins on patched signalling, e.g., which antagonize the cell-cycle inhibitory activity of patched, or (ii) activate or potentiate patched signalling. In other embodiments, the ptc therapeutic can be a hedgehog antagonist. The ptc therapeutic can be, e.g., a peptide, a nucleic acid, a carbohydrate, a small organic molecule, or natural product extract (or fraction thereof).

A “proliferative” form of a hedgehog or ptc therapeutic is one which induces proliferation of adipocyte cells, particularly pre-adipocyte (stem) cells. Conversely, an “antiproliferative” form of a hedgehog or ptc therapeutic is one which inhibits proliferation of an adipocyte cells, preferably in a non-toxic manner, e.g., by promoting or maintaining a differentiated phenotype or otherwise promoting quiescence.

As used herein, “proliferating” and “proliferation” refer to cells undergoing mitosis.

As used herein, “transformed cells” refers to cells which have spontaneously converted to a state of unrestrained growth, i.e., they have acquired the ability to grow through an indefinite number of divisions in culture. Transformed cells may be characterized by such terms as neoplastic, anaplastic and/or hyperplastic, with respect to their loss of growth control.

As used herein, “immortalized cells” refers to cells which have been altered via chemical and/or recombinant means such that the cells have the ability to grow through an indefinite number of divisions in culture.

A “patient” or “subject” to be treated by the subject method can mean either a human or non-human animal.

An “effective amount” of, e.g., a hedgehog therapeutic, with respect to the subject method of treatment, refers to an amount of, e.g., a hedgehog polypeptide in a preparation which, when applied as part of a desired dosage regimen brings about a change in the rate of cell proliferation and/or the state of differentiation of a cell so as to produce an amount of adipocyte cell proliferation or differentiation according to clinically acceptable standards for the disorder to be treated or the cosmetic purpose.

The “growth state” of a cell refers to the rate of proliferation of the cell and the state of differentiation of the cell.

“Homology” and “identity” each refer to sequence similarity between two polypeptide sequences, with identity being a more strict comparison. Homology and identity can each be determined by comparing a position in each sequence which may be aligned for purposes of comparison. When a position in the compared sequence is occupied by the same amino acid residue, then the polypeptides can be referred to as identical at that position; when the equivalent site is occupied by the same amino acid (e.g., identical) or a similar amino acid (e.g., similar in steric and/or electronic nature), then the molecules can be refered to as homologous at that position. A percentage of homology or identity between sequences is a function of the number of matching or homologous positions shared by the sequences. An “unrelated” or “non-homologous” sequence shares less than 40 percent identity, though preferably less than 25 percent identity, with an hedgeog sequence of the present invention.

The term “corresponds to”, when referring to a particular polypeptide or nucleic acid sequence is meant to indicate that the sequence of interest is identical or homologous to the reference sequence to which it is said to correspond.

The terms “recombinant protein”, “heterologous protein” and “exogenous protein” are used interchangeably throughout the specification and refer to a polypeptide which is produced by recombinant DNA techniques, wherein generally, DNA encoding the polypeptide is inserted into a suitable expression construct which is in turn used to transform a host cell to produce the heterologous protein. That is, the polypeptide is expressed from a heterologous nucleic acid.

A “chimeric protein” or “fusion protein” is a fusion of a first amino acid sequence encoding a hedgehog polypeptide with a second amino acid sequence defining a domain foreign to and not substantially homologous with any domain of hh protein. A chimeric protein may present a foreign domain which is found (albeit in a different protein) in an organism which also expresses the first protein, or it may be an “interspecies”, “intergenic”, etc. fusion of protein structures expressed by different kinds of organisms. In general, a fusion protein can be represented by the general formula (X)ₙ-(hh)ₘ-(Y)ₙ, wherein hh represents all or a portion of the hedgehog protein, X and Y each independently represent an amino acid sequences which are not naturally found as a polypeptide chain contiguous with the hedgehog sequence, m is an integer greater than or equal to 1, and each occurrence of n is, independently, 0 or an integer greater than or equal to 1 (n and m are preferably no greater than 5 or 10).

The term “PPARγ” refers to members of the peroxisome proliferator-activated receptors family which are expressed, inter alia, in adipocytic and hematopoietic cells (Braissant, O. et al. Endocrinology 137(1): 354-66), and which function as key regulators of differentiation. Contemplated within this definition are variants thereof, as for example, PPARγ1 and PPARγ2 which are two isoforms having a different N-terminal generated by alternate splicing of a primary RNA transcript (Tontonoz, P. et al. (1994), Genes & Dev. 8:1224-34; Zhu et al. (1993) J. Biol. Chem. 268: 26817-20).

The terms “PPARγ-responsive hyperproliferative cell” and “PPARγ-responsive neoplastic cell” are used interchangeably herein and refer to a neoplastic cell which is responsive to PPARγ agonists. This neoplastic cell responds to PPARγ receptor activation by inhibiting cell proliferation and/or inducing the expression of differentiation-specific genes. This term includes tumor-derived cells that differentiate into adipocytic lineages in response to PPARγ ligands, e.g., human liposarcoma cells.

The term “activation of PPAR” refers to the ability of a compound to selectively activate PPAR-dependent gene expression, e.g., by increasing PPAR-dependent transcription of a gene. Likewise, the term “inhibition of PPAR” refers to the ability of a compound to selectively inhibit PPAR-dependent gene expression, e.g., by decreasing PPAR-dependent transcription of a gene.

The common medical meaning of the term “neoplasia” refers to “new cell growth” that results as a loss of responsiveness to normal growth controls, e.g. to neoplastic cell growth. A “hyperplasia” refers to cells undergoing an abnormally high rate of growth. However, as used herein, the terms neoplasia and hyperplasia can be used interchangably, as their context will reveal, referring generally to cells experiencing abnormal cell growth rates. Neoplasias and hyperplasias include “tumors,” which may be either benign, premalignant or malignant.

As used herein, the terms “hyperproliferative” and “neoplastic” are used interchangeably, and refer to those cells an abnormal state or condition characterized by rapid proliferation or neoplasm. The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth.

The term “adipose cell tumor” refers to all cancers or neoplasias arising from cells of adipocytic lineage, e.g., arising from adipose or adipose precursor cells. The adipose cell tumors include both common and uncommon, benign and malignant lesions, such as lipoma, intramuscular and intermuscular lipoma, neural fibrolipoma, lipoblastoma, lipomatosis, hibernoma, hemangioma and liposarcoma, as well as lesions that may mimic fat-containing soft-tissue masses.

The terms “antineoplastic agent” and “antiproliferative agent” are used interchangeably herein and refer to hedgehog or ptc therapeutic agents that have the functional property of inhibiting the proliferation of PPAR-responsive cells, e.g., inhibiting the development or progression of a neoplasm having such a characteristic, particularly an adipocytic neoplasm or hematopoietic neoplasm.

### III. Exemplary Applications of Method and Compositions

The subject method has wide applicability to the treatment or prophylaxis of disorders afflicting adipocyte tissue. In general, the method can be characterized as including a step of administering to an animal an amount of a ptc or hedgehog therapeutic effective to alter the proliferative state of a treated adipocyte tissue. The mode of administration and dosage regimens will vary depending on the adipocyte tissue(s) which is to be treated. Likewise, as described in further detail below, the use of a particular ptc or hedgehog therapeutic, e.g., an agonist or antagonist, will depend on whether proliferation of cells of the treated tissue is desired or intended to be prevented.

In one aspect, the invention is used to inhibit adipocyte differentiation in mammals. Such aspects of the present invention are thus directed to a method for inhibiting the differentiation of adipocyte precursor cells in a mammal (e.g., inhibiting differentiation of preadipocytes into adipocytes), and comprise administering to the mammal an effective amount of a hedgehog polypeptide or agonist thereof. In such embodiments, the hedgehog proteins and agonists of the present invention can be use to treat (reduce the severity of or ameliorate) body weight disorders which may include, for example, inhibition of adipose cell differentiation and an inhibition of the ability of adipocytes to synthesize fat, e.g., treatment of obesity or of disorders related to abnormal proliferation of adipocytes.

In certain embodiments, the subject method can be used to inhibit the differentiation of preadipocytes to adipocytes, therefore limiting the possibility of cellulite appearing.

In other embodiments, the subject method can be used in livestock to repartition nutrients between subcutaneous fat and other carcass components, including muscle, skin, bone and certain organs, e.g., by administration in the form of a veterinarian composition or as part of a livestock feed.

In another aspect, this invention features methods for inhibiting the proliferation of pre-adipocytes, e.g., inducing differentiation of preadipocytes into adipocytes, by inhibiting a hedgehog-mediated signal transduction pathway. The present results suggest that certain adipocytic cancers may be the result of over-expression of hedgehog, or a loss-of-function of patched or a gain-of-function of smoothened, or some other mutation which mimics the proliferative activity of hedgehog on pre-adipocytes. Thus, the present invention specifically contemplates the use of the subject method for reversing the transformed phenotype of PPARγ-responsive hyperproliferative cells by contacting the cells with a hedgehog antagonists.

In general, the method includes a step of contacting pathological of PPARγ-responsive hyperproliferative cells with an amount of a hedgehog antagonist effective for promoting the differentiation of the hyperproliferative cells. The present method can be performed on cells in culture, e.g., in vitro or ex vivo, or can be performed on cells present in an animal subject, e.g., as part of an in vivo therapeutic protocol. The therapeutic regimen can be carried out on a human or other animal subject. Induction of terminal differentiation of transformed cells in vivo in response to hedgehog antagonists represents a promising alternative to conventional highly toxic regimens of chemotherapy.

In one embodiment, the cells to be treated are hyperproliferative cells of adipocytic lineage, e.g., arising from adipose or adipose precursor cells. For instance, the instant method can be carried out to prevent the proliferation of an adipose cell tumor. The adipose tumor cells can be of a liposarcoma. The term “liposarcoma” is recognized by those skilled in the art and refers to a malignant tumor characterized by large anaplastic lipoblasts, sometimes with foci of normal fat cells. Exemplary liposarcoma types which are can be treated by the present invention include, but are not limited to, well differentiated/dedifferentiated, myxoid/round cell and pleiomorphic (reviewed in Sreekantaiah, C. et al., (1994) supra).

Another adipose cell tumor which may be treated by the present method include lipomas, e.g., benign fatty tumors usually composed of mature fat cells. Likewise, the method of the present invention can be used in the treatment and/or prophylaxis of lipochondromas, lipofibromas and lipogranulomas. Lipochondroma are tumors composed of mature lipomatous and cartilaginous elements; lipofibromas are lipomas containing areas of fibrosis; and lipogranuloma are characterized by nodules of lipoid material associated with granulomatous inflammation.

The subject method can also be useful in treating malignancies of the various organ systems, such as those affecting lung, breast, lymphoid, gastrointestinal, and genito-urinary tract as well as adenocarcinomas which include malignancies such as most colon cancers, renal-cell carcinoma, prostate cancer and/or testicular tumors, non-small cell carcinoma of the lung, cancer of the small intestine and cancer of the esophagus. According to the general paradigm of PPAR involvement in differentiation of transformed cells, exemplary solid tumors that can be treated according to the method of the present invention include sarcomas and carcinomas with PPAR-responsive phenotypes, such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

While the hedgehog antagonists can be utilized alone, the subject differentiation therapy can be combined with other therapeutics, e.g., such as cell cycle inhibitors, agents which promote apoptosis, PPARγ ligands, agents which strengthen the immune response, and/or RxR agonists. Some of the co-administered therapeutics, particular those with cytotoxic effects or which lack specficity for the treated cells, may be given in smaller doses due to an additive, and sometimes synergistic effect with the hedgehog antagonist.

For instance, the subject method may involve, in addition to the use of hedgehog antagonists, one or more other anti-tumor substances. Exemplary combinatorial therapies combining with hedgehog antagonists include the use of such as agents as: mitotic inhibitors, such as vinblastine; alkylating agents, such as cisplatin, carboplatin and cyclophosphamide; antimetabolites, such as 5-fluorouracil, cytosine arabinoside, hydroxyurea or N-[5-[N-(3,4-dihydro-2-methyl-4-oxoquinazolin-6-ylmethyl)-N-methylamino]-2-thenoyl]-L-glutamic acid; intercalating antibiotics, as for example adriamycin and bleomycin; enzymes, such as asparaginase; topoisomerase inhibitors, such as etoposide; biological response modifiers, e.g., to enhance anti-tumor responses, such as interferon; apoptotic agents, such as actinomycin D; and anti-hormones, for example antioestrogens such as tamoxifen or, for example antiandrogens such as 4′-cyano-3-(4-fluorophenylsulphonyl)-2-hydroxy-2-methyl-3′-(trifluoromethyl) propionanilide. Such conjoint treatment may be achieved by way of the simultaneous, sequential or separate dosing of the individual components of the treatment.

In another embodiment, the subject hedgehog antagonist is conjointly administered with a PPARγ ligand. Particular examples of a non-naturally occurring PPARγ ligand include thiazolidine (TZD) derivatives known as thiazolidinediones, e.g., proglitazone (also known as AD-4833 and U-72107E), troglitazone (also known as CS-045) (Sankyo) and C1-991 (Parke-Davis), BRL 49653, ciglitazone, englitazone and chemical derivatives thereof. These compounds are conventionally known for the treatment of diabetes. See e.g., U.S. Pat. Nos. 4,812,570; 4,775,687; 4,725,610; 4,582,839; and 4,572,912 for exemplary sources of such compounds. U.S. Pat. No. 5,521,201 and European Patent Applications 0008203, 0139421, 0155845, 0177353, 0193256, 0207581 and 0208420, and Chem. Pharm. Bull 30 (10) 3580-3600 relate to thiazolidinedione derivatives, and describe commercial sources/synthetic schemes for a variety of TZD and TZD-like analogs, which may be useful in carrying out the method of the present invention. Particular examples of naturally-occurring PPARγ ligands include arachidonic acid metabolites, e.g., prostaglandin J₂ (PGJ₂) metabolites, e.g., 15-deoxy-Δ^{12,14}-prostaglandin J₂. Prostaglandin J2 dehydration and isomerization products, including Δ¹²-PGJ₂ and 15-deoxy-Δ^{12,14}-PGJ₂ have been shown to occur by incubation of prostaglandin D₂ (PGD₂) in the presence of human plasma or human serum albumin (Fitzpatrick and Wyvalda (1983) J. Biol. Chem. 258:11713-18). Δ¹²-PGJ₂ has been shown to be a significant PGD₂ metabolite present in human and monkey urine, indicating that PGJ₂ metabolites are also found in vivo (Hirata et al. (1994) PNAS USA 91:11192-96).

Also contemplated are chemicals that stimulate the endogenous production of arachidonic acid metabolites, when administered systemically or in vitro. Enhanced production of endogenous arachidonic acid metabolites may occur by stimulating at least one of the release of arachidonic acid from precursor glycerophospholipids, the oxygenation of free arachidonic acid by a cyclo-oxygenase enzyme, and the metabolism of prostaglandin H₂ to a specific biologically active prostaglandin metabolite (reviewed in Smith, W. (1989) Biochem. J., 259:315-24).

In general, it will be preferable to choose a PPAR agonist which specifically activates that PPAR isoform relative to, for example, PPAR and/or PPAR. According to this present invention, specificity for the PPAR isoform can reduce unwanted side effects, such as PPAR-mediated hepatocarcinogenesis. In particular, the PPAR agonist of the present method preferably activates PPAR-dependent transcription at a concentration at least 1 order of magnitude less than that which activates PPAR-dependent transcription, and even more preferably at a concentration at least 2, 3, 4 or 5 orders of magnitude less. PPARγ ligands useful for practicing the present invention, and methods of making these compounds are known. Examples of PPAR agonists are disclosed in PCT publications WO 91/07107; WO 92/02520; WO 94/01433; WO 89/08651; WO 95/18533; WO 95/35108; Japanese patent publication 69383/92; and U.S. Pat. Nos. 5,523,314; 5,521,202; 5,510,360; 5,498,621; 5,496,621; 5,494,927; 5,480,896; 5,478,852; 5,468,762; 5,464,856; 5,457,109; 4,287,200; 4,340,605; 4,438,141; 4,444,779; 4,461,902; 4,572,912; 4,687,777; 4,703,052; 4,725,610; 4,873,255; 4,897,393; 4,897,405; 4,918,091; 4,948,900; 5,002,953; 5,061,717; 5,120,754; 5,132,317; 5,194,443; 5,223,522; 5,232,925; and 5,260,445. Exemplary PPAR agonist can be selected from amongst such compounds as 5-[4-[2-(5-ethylpyridin-2-yl)ethoxyl]benzyl]thiadiazolidine-2,4-dione: (pioglitazone); 5-[4-[(1-methylcyclohexyl)methoxy]benzyl]thiadiazolidine-2,4-dione: (ciglitazone); 5-[(2-benzyl-2,3-dihydrobenzopyran)-5-ylmethyl]thiadiazoline-2,4-dione: (englitazone); 5-[(2-alkoxy-5-pyridyl)methyl]-2,4-thiazolidinedione; 5-[(substituted-3-pyridyl)methyl]-2,4-thiazolidinedione; 5-[4-(2-methyl-2-phenylpropoxy)benzyl]thiazolidine-2,4-dione; 5-[4-[3-(4-methoxyphenyl)-2-oxooxazolidin-5-yl]-methoxy]benzyl-2,4-thiazolidinedione; 5-[4-[3-(3,4-difluorophenyl)-2-oxooxazolidin-5-yl]-methoxy]benzyl-2,4-thiazolidinedione; 5-[4-[3-(4-chloro-2-fluorophenyl)-2-oxooxazolidin-5-yl]methoxy]benzyl-2,4-thiazolidinedione; 5-[4-[3-(4-trifluoromethoxyphenyl)-2-oxooxazolidin-5-yl]methoxy]benzyl-2,4-thiazolidinedione; 5-[4-[3-(4-trifluoromethylphenyl)-2-oxooxazolidin-5-yl]methoxy]benzyl-2,4-thiazolidinedione; 5-[4-[2-[3-(4-trifluoromethylphenyl)-2-oxooxazolidin-5-yl]ethoxy]benzyl]-2,4-thiazolidinedione; 5-[4-[2-[3-(4-chloro-2-fluorophenyl)-2-oxooxazolidin-5-yl]ethoxy]benzyl]-2,4-thiazolidinedione; 5-[4-[3-(4-pyridyl)-2-oxooxazolidin-5-yl]methoxy]benzyl-2,4-thiazolidinedione; 5-[[4-[(3,4-dihydro-6-hydroxy-2,5,7,8-tetramethyl-2H-1-benzopyran-2-yl)methoxy]phenyl]methyl]-2,4-thiazolidinedione: (troglitazone); 4-(2-naphthylmethyl)-1,2,3,5-oxathiadiazole-2-oxide; 5-[4-[2-[N-(benzoxazol-2-yl)-N-methylamino]ethoxy]benzyl]-5-methylthiazolidine-2,4-dione; 5-[4-[2-[2,4-dioxo-5-phenylthiazolidin-3-yl)ethoxy]benzyl]thiazolidine-2,4-dione; 5-[4-[2-[N-methyl-N-(phenoxycarbonyl)amino]ethoxy]benzyl]thiazolidine-2,4-dione; 5-[4-(2-phenoxyethoxy)benzyl]thiazolidine-2,4-dione; 5-[4-[2-(4-chlorophenyl)ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxy-1-methylcyclohexyl)methoxy]benzyl]thiadiazolidine-2,4-dione; 5-[4-[2-(5-methyl-2-phenyloxazol-4-yl)ethoxyl]benzyl]thiadizolidione-2,4-dione; 5-[[2-(2-naphthylmethyl)benzoxazol]-5-ylmethyl]thiadiazoline-2,4-dione; 5-[4-[2-(3-phenylureido)ethoxyl]benzyl]thiadiazoline-2,4-dione; 5-[4-[2-[N-(benzoxazol-2-yl)-N-methylamino]ethoxy]benzy]thiadiazoline-2,4-dione; 5-[4-[3-(5-methyl-2-phenyloxazol-4-yl)propionyl]benzyl]thiadiazoline-2,4-dione; 5-[2-(5-methyl-2-phenyloxazole-4-yl)propionyl]benzyl]thiadiazoline-2,4-dione; 5-[2-(5-methyl-2-phenyloxazol-4-ylmethyl)benzofuran-5-ylmethyl]-oxazolidine-2,4-dione; 5-[4-[2-[N-methyl-N-(2-pyridyl)amino]ethoxy]benzyl]thiazolidine-2,4-dione; and 5-[4-[2-[N-(benzoxazol-2-yl)-N-methylamino]ethoxy]benzyl]-oxazolidine-2,4-dione.

In another embodiment, the subject methods combines the use of hedgehog antagonists in combination with one or more RxR-specific ligands. For instance, the subject method can be practiced by conjoint treatment using a hedgehog antagonist as described above and an RxR agonist such as a natural and/or synthetic retinoid. A wide variety of RxR ligands appropriate for use in the subject method are known in the art. Exemplary natural RxR ligands include all-trans-retinoic acid and phytanic acid. Exemplary synthetic RxR ligands include 9-cis-retinoic acid, LG268, AGN191701, SR11217, SR11237, SR11236, SR11246, SR11249 SR11256, LGD1069, various tricyclic retinoids, teravinyl-alkadi- or trienoic derivatives of retinoids, and phenyl-methyl heterocylic and tetrahydro-napthyl analogs of retinoic acid (c.f., Apfel et al. (1995) JBC 270:30765; Minucci et al. (1996) PNAS 93:1803; Hembree et al. (1996) Cancer Res 56:1794; Kizaki et al. (1996) Blood 87:1977; Lemotte et al. (1996) Eur J Biochem 236:328; and U.S. Pat. Nos. 5,552,271; 5,466,861; 5,514,821; PCT publications WO 96/05165; WO 96/20914; WO 94/15901; WO 93/21146; and European Patent publication EP 0694301.

To further illustrate, the RxR ligand can be a compound represented in the general formula:

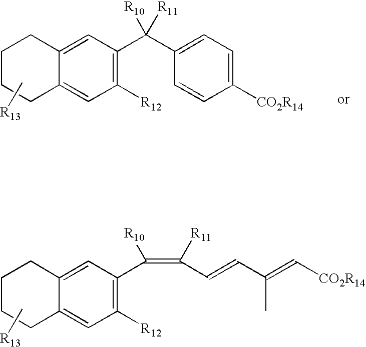

d U.S. Pat. No. 5,466,861.

The two (or more) compounds are administered in combination according to the invention. The terms “in combination” and “conjointly” in this context means that the drugs are given substantially contemporaneously, either simultaneously or sequentially. If given sequentially, at the onset of administration of the second compound, the first of the two compounds is preferably still detectable at effective concentrations at the site of treatment.

The maintenance of tissues and organs ex vivo is also highly desirable. Tissue replacement therapy is well established in the treatment of human disease. There are many situations where one may wish to transplant adipocyte cells, especially adipocyte stem cells, into a recipient host where the recipient's cells are missing, damaged or dysfunctional. The subject method can be used to regulate the growth of adipocyte cells and tissue in vitro, as well as to accelerate the grafting of impanted adipocyte tissue to an animal host

In this regard, the present invention also concerns adipocyte cultures which have been expanded by treatment with a hedgehog or other ptc therapeutic. In an illustrative embodiment, such a method comprises obtaining a adipocyte sample, preferably one including pre-adipocytes; optionally treating the cell sample enzymically to separate the cells; culturing, in the presence of a hedgehog or ptc therapeutic.

### IV. Exemplary Hedgehog Therapeutic Compounds

The hedgehog therapeutic compositions of the subject method can be generated by any of a variety of techniques, including purification of naturally occurring proteins, recombinantly produced proteins and synthetic chemistry. Polypeptide forms of the hedgehog therapeutics are preferably derived from vertebrate hedgehog proteins, e.g., have sequences corresponding to naturally occurring hedgehog proteins, or fragments thereof, from vertebrate organisms. However, it will be appreciated that the hedgehog polypeptide can correspond to a hedgehog protein (or fragment thereof) which occurs in any metazoan organism.

The various naturally-occurring hedgehog proteins from which the subject therapeutics can be derived are characterized by a signal peptide, a highly conserved N-terminal region, and a more divergent C-terminal domain. In addition to signal sequence cleavage in the secretory pathway (Lee, J. J. et al. (1992) Cell 71:33-50; Tabata, T. et al. (1992) Genes Dev. 2635-2645; Chang, D. E. et al. (1994) Development 120:3339-3353), hedgehog precursor proteins naturally undergo an internal autoproteolytic cleavage which depends on conserved sequences in the C-terminal portion (Lee et al. (1994) Science 266:1528-1537; Porter et al. (1995) Nature 374:363-366). This autocleavage leads to a 19 kD N-terminal peptide and a C-terminal peptide of 26-28 kD (Lee et al. (1992) supra; Tabata et al (1992) supra; Chang et al. (1994) supra; Lee et al. (1994) supra; Bumcrot, D. A., et al. (1995) Mol. Cell. Biol. 15:2294-2303; Porter et al. (1995) supra; Ekker, S. C. et al. (1995) Curr. Biol. 5:944-955; Lai, C. J. et al. (1995) Development 121:2349-2360). The N-terminal peptide stays tightly associated with the surface of cells in which it was synthesized, while the C-terminal peptide is freely diffusible both in vitro and in vivo (Lee et al. (1994) supra; Bumerot et al. (1995) supra; Mart', E. et al. (1995) Development 121:2537-2547; Roelink, H. et al. (1995) Cell 81:445-455). Cell surface retention of the N-terminal peptide is dependent on autocleavage, as a truncated form of hedgehog encoded by an RNA which terminates precisely at the normal position of internal cleavage is diffusible in vitro (Porter et al. (1995) supra) and in vivo (Porter, J. A. et al. (1996) Cell 86, 21-34). Biochemical studies have shown that the autoproteolytic cleavage of the hedgehog precursor protein proceeds through an internal thioester intermediate which subsequently is cleaved in a nucleophilic substitution. It is suggested that the nucleophile is a small lipophilic molecule, more particularly cholesterol, which becomes covalently bound to the C-terminal end of the N-peptide (Porter et al. (1996) supra), tethering it to the cell surface.

The vertebrate family of hedgehog genes includes at least four members, e.g., paralogs of the single drosophila hedgehog gene (SEQ ID No. 19). Three of these members, herein referred to as Desert hedgehog (Dhh), Sonic hedgehog (Shh) and Indian hedgehog (Ihh), apparently exist in all vertebrates, including fish, birds, and mammals. A fourth member, herein referred to as tiggie-winkle hedgehog (Thh), appears specific to fish. According to the appended sequence listing, (see also Table 1) a chicken Shh polypeptide is encoded by SEQ ID No:1; a mouse Dhh polypeptide is encoded by SEQ ID No:2; a mouse Ihh polypeptide is encoded by SEQ ID No:3; a mouse Shh polypeptide is encoded by SEQ ID No:4 a zebrafish Shh polypeptide is encoded by SEQ ID No:5; a human Shh polypeptide is encoded by SEQ ID No:6; a human Ihh polypeptide is encoded by SEQ ID No:7; a human Dhh polypeptide is encoded by SEQ ID No. 8; and a zebrafish Thh is encoded by SEQ ID No. 9.

TABLE 1
Guide to hedgehog sequences in Sequence Listing

| | Nucleotide | Amino Acid |
|---|---|---|
| Chicken Shh | SEQ ID No. 1 | SEQ ID No. 10 |
| Mouse Dhh | SEQ ID No. 2 | SEQ ID No. 11 |
| Mouse Ihh | SEQ ID No. 3 | SEQ ID No. 12 |
| Mouse Shh | SEQ ID No. 4 | SEQ ID No. 13 |
| Zebrafish Shh | SEQ ID No. 5 | SEQ ID No. 14 |
| Human Shh | SEQ ID No. 6 | SEQ ID No. 15 |
| Human Ihh | SEQ ID No. 7 | SEQ ID No. 16 |
| Human Dhh | SEQ ID No. 8 | SEQ ID No. 17 |
| Zebrafish Thh | SEQ ID No. 9 | SEQ ID No. 18 |
| Drosophila HH | SEQ ID No. 19 | SEQ ID No. 20 |

In addition to the sequence variation between the various hedgehog homologs, the hedgehog proteins are apparently present naturally in a number of different forms, including a pro-form, a full-length mature form, and several processed fragments thereof. The pro-form includes an N-terminal signal peptide for directed secretion of the extracellular domain, while the full-length mature form lacks this signal sequence.

As described above, further processing of the mature form occurs in some instances to yield biologically active fragments of the protein. For instance, sonic hedgehog undergoes additional proteolytic processing to yield two peptides of approximately 19 kDa and 27 kDa, the 19 kDa fragment corresponding to an proteolytic N-terminal portion of the mature protein.

In addition to proteolytic fragmentation, the vertebrate hedgehog proteins can also be modified post-translationally, such as by glycosylation and/or addition of lipophilic moieties, such as stents, fatty acids, etc., though bacterially produced (e.g. unmodified) forms of the proteins still maintain certain of the bioactivities of the native protein. Bioactive fragments of hedgehog polypeptides of the present invention have been generated and are described in great detail in, e.g., PCT publications WO 95/18856 and WO 96/17924.

There are a wide range of lipophilic moieties with which hedgehog polypeptides can be derivatived. The term “lipophilic group”, in the context of being attached to a hedgehog polypeptide, refers to a group having high hydrocarbon content thereby giving the group high affinity to lipid phases. A lipophilic group can be, for example, a relatively long chain alkyl or cycloalkyl (preferably n-alkyl) group having approximately 7 to 30 carbons. The alkyl group may terminate with a hydroxy or primary amine “tail”. To further illustrate, lipophilic molecules include naturally-occurring and synthetic aromatic and non-aromatic moieties such as fatty acids, sterols, esters and alcohols, other lipid molecules, cage structures such as adamantane and buckminsterfullerenes, and aromatic hydrocarbons such as benzene, perylene, phenanthrene, anthracene, naphthalene, pyrene, chrysene, and naphthacene.

In one embodiment, the hedgehog polypeptide is modified with one or more sterol moieties, such as cholesterol. See, for example, PCT publication WO 96/17924. In certain embodiments, the cholesterol is preferably added to the C-terminal glycine were the hedgehog polypeptide corresponds to the naturally-occurring N-terminal proteolytic fragment.

In another embodiment, the hedgehog polypeptide can be modified with a fatty acid moiety, such as a myrostoyl, palnitoyl, stearoyl, or arachidoyl moiety. See, e.g., Pepinsky et al. (1998) J Biol. Chem 273: 14037.

In addition to those effects seen by cholesterol-addition to the C-terminus or fatty acid addition to the N-terminus of extracellular fragments of the protein, at least certain of the biological activities of the hedgehog gene products are unexpectedly potentiated by derivativation of the protein with lipophilic moieties at other sites on the protein and/or by moieties other than cholesterol or fatty acids. Certain aspects of the invention are directed to the use of preparations of hedgehog polypeptides which are modified at sites other than N-terminal or C-terminal residues of the natural processed form of the protein, and/or which are modified at such terminal residues with lipophilic moieties other than a sterol at the C-terminus or fatty acid at the N-terminus.

Particularly useful as lipophilic molecules are alicyclic hydrocarbons, saturated and unsaturated fatty acids and other lipid and phospholipid moieties, waxes, cholesterol, isoprenoids, terpenes and polyalicyclic hydrocarbons including adamantane and buckminsterfullerenes, vitamins, polyethylene glycol or oligoethylene glycol, (C1-C18)-alkyl phosphate diesters, —O—CH2-CH(OH)—O—(C12-C18)-alkyl, and in particular conjugates with pyrene derivatives. The lipophilic moiety can be a lipophilic dye suitable for use in the invention include, but are not limited to, diphenylhexatriene, Nile Red, N-phenyl-1-naphthylamine, Prodan, Laurodan, Pyrene, Perylene, rhodamine, rhodamine B, tetramethylrhodamine, Texas Red, sulforhodamine, 1,1′-didodecyl-3,3,3′,3′tetramethylindocarbocyanine perchlorate, octadecyl rhodamine B and the BODIPY dyes available from Molecular Probes Inc.

Other exemplary lipophilic moietites include aliphatic carbonyl radical groups include 1- or 2-adamantylacetyl, 3-methyladamant-1-ylacetyl, 3-methyl-3-bromo-1-adamantylacetyl, 1-decalinacetyl, camphoracetyl, camphaneacetyl, noradamantylacetyl, norbomaneacetyl, bicyclo[2.2.2.]-oct-5-eneacetyl, 1-methoxybicyclo[2.2.2.]-oct-5-ene-2-carbonyl, cis-5-norbornene-endo-2,3-dicarbonyl, 5-norbomen-2-ylacetyl, (1R)-(−)-myrtentaneacetyl, 2-norbomaneacetyl, anti-3-oxo-tricyclo[2.2.1.0<2,6>]-heptane-7-carbonyl, decanoyl, dodecanoyl, dodecenoyl, tetradecadienoyl, decynoyl or dodecynoyl.

The hedgehog polypeptide can be linked to the hydrophobic moiety in a number of ways including by chemical coupling means, or by genetic engineering.

There are a large number of chemical cross-linking agents that are known to those skilled in the art. For the present invention, the preferred cross-linking agents are heterobifunctional cross-linkers, which can be used to link the hedgehog polypeptide and hydrophobic moiety in a stepwise manner. Heterobifunctional cross-linkers provide the ability to design more specific coupling methods for conjugating to proteins, thereby reducing the occurrences of unwanted side reactions such as homo-protein polymers. A wide variety of heterobifunctional cross-linkers are known in the art. These include: succinimidyl 4-(N-maleimidomethyl) cyclohexane-1-carboxylate (SMCC), m-Maleimidobenzoyl-N-hydroxysuccinimide ester (MBS); N-succinimidyl (4-iodoacetyl)aminobenzoate (SIAB), succinimidyl 4-(p-maleimidophenyl)butyrate (SMPB), 1-ethyl-3-(3-dimethylaminopropyl)carbodiimide hydrochloride (EDC); 4-succinimidyloxycarbonyl-a-methyl-a-(2-pyridyldithio)-tolune (SMPT), N-succinimidyl 3-(2-pyridyldithio) propionate (SPDP), succinimidyl 6-[3-(2-pyridyldithio)propionate]hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo.

In addition to the heterobifunctional cross-linkers, there exists a number of other cross-linking agents including homobifunctional and photoreactive cross-linkers. Disuccinimidyl suberate (DSS), bismaleimidohexane (BMH) and dimethylpimelimidate.2 HCl (DMP) are examples of useful homobifunctional cross-linking agents, and bis-[β-(4-azidosalicylamido)ethyl]disulfide (BASED) and N-succinimidyl-6(4′-azido-2′-nitrophenylamino)hexanoate (SANPAH) are examples of useful photoreactive cross-linkers for use in this invention. For a recent review of protein coupling techniques, see Means et al. (1990) Bioconjugate Chemistry 1:2-12, incorporated by reference herein.

One particularly useful class of heterobifunctional cross-linkers, included above, contain the primary amine reactive group, N-hydroxysuccinimide (NHS), or its water soluble analog N-hydroxysulfosuccinimide (sulfo-NHS). Primary amines (lysine epsilon groups) at alkaline pH's are unprotonated and react by nucleophilic attack on NHS or sulfo-NHS esters. This reaction results in the formation of an amide bond, and release of NHS or sulfo-NHS as a by-product.

Another reactive group useful as part of a heterobifunctional cross-linker is a thiol reactive group. Common thiol reactive groups include maleimides, halogens, and pyridyl disulfides. Maleimides react specifically with free sulfhydryls (cysteine residues) in minutes, under slightly acidic to neutral (pH 6.5-7.5) conditions. Halogens (iodoacetyl functions) react with —SH groups at physiological pH's. Both of these reactive groups result in the formation of stable thioether bonds.

The third component of the heterobifunctional cross-linker is the spacer arm or bridge. The bridge is the structure that connects the two reactive ends. The most apparent attribute of the bridge is its effect on steric hindrance. In some instances, a longer bridge can more easily span the distance necessary to link two complex biomolecules. For instance, SMPB has a span of 14.5 angstroms.

Preparing protein-protein conjugates using heterobifunctional reagents is a two-step process involving the amine reaction and the sulfhydryl reaction. For the first step, the amine reaction, the protein chosen should contain a primary amine. This can be lysine epsilon amines or a primary alpha amine found at the N-terminus of most proteins. The protein should not contain free sulfhydryl groups. In cases where both proteins to be conjugated contain free sulfhydryl groups, one protein can be modified so that all sulfhydryls are blocked using for instance, N-ethylmaleimide (see Partis et al. (1983) J. Pro. Chem. 2:263, incorporated by reference herein). Ellman's Reagent can be used to calculate the quantity of sulfhydryls in a particular protein (see for example Ellman et al. (1958) Arch. Biochem. Biophys. 74:443 and Riddles et al. (1979) Anal. Biochem. 94:75, incorporated by reference herein).

The reaction buffer should be free of extraneous amines and sulfhydryls. The pH of the reaction buffer should be 7.0-7.5. This pH range prevents maleimide groups from reacting with amines, preserving the maleimide group for the second reaction with sulfhydryls.

The NHS-ester containing cross-linkers have limited water solubility. They should be dissolved in a minimal amount of organic solvent (DMF or DMSO) before introducing the cross-linker into the reaction mixture. The cross-linker/solvent forms an emulsion which will allow the reaction to occur.

The sulfo-NHS ester analogs are more water soluble, and can be added directly to the reaction buffer. Buffers of high ionic strength should be avoided, as they have a tendency to “salt out” the sulfo-NHS esters. To avoid loss of reactivity due to hydrolysis, the cross-linker is added to the reaction mixture immediately after dissolving the protein solution.

The reactions can be more efficient in concentrated protein solutions. The more alkaline the pH of the reaction mixture, the faster the rate of reaction. The rate of hydrolysis of the NHS and sulfo-NHS esters will also increase with increasing pH. Higher temperatures will increase the reaction rates for both hydrolysis and acylation.

Once the reaction is completed, the first protein is now activated, with a sulfhydryl reactive moiety. The activated protein may be isolated from the reaction mixture by simple gel filtration or dialysis. To carry out the second step of the cross-linking, the sulfhydryl reaction, the lipophilic group chosen for reaction with maleimides, activated halogens, or pyridyl disulfides must contain a free sulfhydryl. Alternatively, a primary amine may be modified with to add a sulfhydryl

In all cases, the buffer should be degassed to prevent oxidation of sulfhydryl groups. EDTA may be added to chelate any oxidizing metals that may be present in the buffer. Buffers should be free of any sulfhydryl containing compounds.

Maleimides react specifically with -SH groups at slightly acidic to neutral pH ranges (6.5-7.5). A neutral pH is sufficient for reactions involving halogens and pyridyl disulfides. Under these conditions, maleimides generally react with —SH groups within a matter of minutes. Longer reaction times are required for halogens and pyridyl disulfides.

The first sulfhydryl reactive-protein prepared in the amine reaction step is mixed with the sulfhydryl-containing lipophilic group under the appropriate buffer conditions. The conjugates can be isolated from the reaction mixture by methods such as gel filtration or by dialysis.

Exemplary activated lipophilic moieties for conjugation include: N-(1-pyrene)maleimide; 2,5-dimethoxystilbene-4′-maleimide, eosin-5-maleimide; fluorescein-5-maleimide; N-(4-(6-dimethylamino-2-benzofuranyl)phenyl)maleimide; benzophenone-4-maleimide; 4-dimethylaminophenylazophenyl-4′-maleimide (DABMI), tetramethylrhodamine-5-maleimide, tetramethylrhodamine-6-maleimide, Rhodamine Red™ C2 maleimide, N-(5-aminopentyl)maleimide, trifluoroacetic acid salt, N-(2-aminoethyl)maleimide, trifluoroacetic acid salt, Oregon Green™ 488 maleimide, N-(2-((2-(((4-azido-2,3,5,6-tetrafluoro)benzoyl)amino)ethyl)dithio)ethyl)maleimide (TFPAM-SS1), 2-(1-(3-dimethylaminopropyl)-indol-3-yl)-3-(indol-3-yl) maleimide (bisindolylmaleimide; GF 109203X), BODIPY® FL N-(2-aminoethyl)maleimide, N-(7-dimethylamino-4-methylcoumarin-3-yl)maleimide (DACM), Alexa™ 488 C5 maleimide, Alexa™ 594 C5 maleimide, sodium saltN-(1-pyrene)maleimide, 2,5-dimethoxystilbene-4′-maleimide, eosin-5-maleimide, fluorescein-5-maleimide, N-(4-(6-dimethylamino-2-benzofuranyl)phenyl)maleimide, benzophenone-4-maleimide, 4-dimethylaminophenylazophenyl-4′-maleimide, 1-(2-maleimidylethyl)-4-(5-(4-methoxyphenyl)oxazol-2-yl)pyridinium methanesulfonate, tetramethylrhodamine-5-maleimide, tetramethylrhodamine-6-maleimide, Rhodamine Red™ C2 maleimide, N-(5-aminopentyl)maleimide, N-(2-aminoethyl)maleimide, N-(2-((2-(((4-azido-2,3,5,6-tetrafluoro)benzoyl)amino)ethyl)dithio)ethyl)maleimide, 2-(1-(3-dimethylaminopropyl)-indol-3-yl)-3-(indol-3-yl) maleimide, N-(7-dimethylamino-4-methylcoumarin-3-yl)maleimide (DACM), 11H-Benzo[a]fluorene, Benzo[a]pyrene.

In one embodiment, the hedgehog polypeptide can be derivatived using pyrene maleimide, which can be purchased from Molecular Probes (Eugene, Oreg.), e.g., N-(1-pyrene)maleimide or 1-pyrenemethyl iodoacetate (PMIA ester).

For those embodiments wherein the hydophobic moiety is a polypeptide, the modified hedgehog polypeptide of this invention can be constructed as a fusion protein, containing the hedgehog polypeptide and the hydrophobic moiety as one contiguous polypeptide chain.

In certain embodiments, the lipophilic moiety is an amphipathic polypeptide, such as magainin, cecropin, attacin, melittin, gramicidin S, alpha-toxin of Staph. aureus, alamethicin or a synthetic amphipathic polypeptide. Fusogenic coat proteins from viral particles can also be a convenient source of amphipathic sequences for the subject hedgehog proteins

Moreover, mutagenesis can be used to create modified hh polypeptides, e.g., for such purposes as enhancing therapeutic or prophylactic efficacy, or stability (e.g., ex vivo shelf life and resistance to proteolytic degradation in vivo). Such modified peptides can be produced, for instance, by amino acid substitution, deletion, or addition. Modified hedgehog polypeptides can also include those with altered post-translational processing relative to a naturally occurring hedgehog protein, e.g., altered glycosylation, cholesterolization, prenylation and the like.

In one embodiment, the hedgehog therapeutic is a polypeptide encodable by a nucleotide sequence that hybridizes under stringent conditions to a hedgehog coding sequence represented in one or more of SEQ ID Nos:1-9 or 19. Appropriate stringency conditions which promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C.

As described in the literature, genes for other hedgehog proteins, e.g., from other animals, can be obtained from mRNA or genomic DNA samples using techniques well known in the art. For example, a cDNA encoding a hedgehog protein can be obtained by isolating total mRNA from a cell, e.g. a mammalian cell, e.g. a human cell, including embryonic cells. Double stranded cDNAs can then be prepared from the total mRNA, and subsequently inserted into a suitable plasmid or bacteriophage vector using any one of a number of known techniques. The gene encoding a hedgehog protein can also be cloned using established polymerase chain reaction techniques.

Preferred nucleic acids encode a hedgehog polypeptide comprising an amino acid sequence at least 60% homologous or identical, more preferably 70% homologous or identical, and most preferably 80% homologous or identical with an amino acid sequence selected from the group consisting of SEQ ID Nos:10-18 or 20. Nucleic acids which encode polypeptides at least about 90%, more preferably at least about 95%, and most preferably at least about 98-99% homology or identity with an amino acid sequence represented in one of SEQ ID Nos:10-18 or 20 are also within the scope of the invention.

In addition to native hedgehog proteins, hedgehog polypeptides preferred by the present invention are at least 60% homologous or identical, more preferably 70% homologous or identical and most preferably 80% homologous or identical with an amino acid sequence represented by any of SEQ ID Nos:10-18 or 20. Polypeptides which are at least 90%, more preferably at least 95%, and most preferably at least about 98-99% homologous or identical with a sequence selected from the group consisting of SEQ ID Nos:10-18 or 20 are also within the scope of the invention. The only prerequisite is that the hedgehog polypeptide is capable of modulating the growth of adipocyte cells.

The term “recombinant protein” refers to a polypeptide of the present invention which is produced by recombinant DNA techniques, wherein generally, DNA encoding a hedgehog polypeptide is inserted into a suitable expression vector which is in turn used to transform a host cell to produce the heterologous protein. Moreover, the phrase “derived from”, with respect to a recombinant hedgehog gene, is meant to include within the meaning of “recombinant protein” those proteins having an amino acid sequence of a native hedgehog protein, or an amino acid sequence similar thereto which is generated by mutations including substitutions and deletions (including truncation) of a naturally occurring form of the protein.

The method of the present invention can also be carried out using variant forms of the naturally occurring hedgehog polypeptides, e.g., mutational variants.

As is known in the art, hedgehog polypeptides can be produced by standard biological techniques or by chemical synthesis. For example, a host cell transfected with a nucleic acid vector directing expression of a nucleotide sequence encoding the subject polypeptides can be cultured under appropriate conditions to allow expression of the peptide to occur. The polypeptide hedgehog may be secreted and isolated from a mixture of cells and medium containing the recombinant hedgehog polypeptide. Alternatively, the peptide may be retained cytoplasmically by removing the signal peptide sequence from the recombinant hedgehog gene and the cells harvested, lysed and the protein isolated. A cell culture includes host cells, media and other byproducts. Suitable media for cell culture are well known in the art. The recombinant hedgehog polypeptide can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such peptide. In a preferred embodiment, the recombinant hedgehog polypeptide is a fusion protein containing a domain which facilitates its purification, such as an hedgehog/GST fusion protein. The host cell may be any prokaryotic or eukaryotic cell.

Recombinant hedgehog genes can be produced by ligating nucleic acid encoding an hedgehog protein, or a portion thereof, into a vector suitable for expression in either prokaryotic cells, eukaryotic cells, or both. Expression vectors for production of recombinant forms of the subject hedgehog polypeptides include plasmids and other vectors. For instance, suitable vectors for the expression of a hedgehog polypeptide include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al. (1983) in Experimental Manipulation of Gene Expression, ed. M. Inouye Academic Press, p. 83, incorporated by reference herein). These vectors can replicate in E. coli due to the presence of the pBR322 ori, and in S. cerevisiae due to the replication determinant of the yeast 2 micron plasmid. In addition, drug resistance markers such as ampicillin can be used. In an illustrative embodiment, an hedgehog polypeptide is produced recombinantly utilizing an expression vector generated by sub-cloning the coding sequence of one of the hedgehog genes represented in SEQ ID Nos:1-10.

The preferred mammalian expression vectors contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and phyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papillomavirus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press: 1989) Chapters 16 and 17.

In some instances, it may be desirable to express the recombinant hedgehog polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

When it is desirable to express only a portion of an hedgehog protein, such as a form lacking a portion of the N-terminus, i.e. a truncation mutant which lacks the signal peptide, it may be necessary to add a start codon (ATG) to the oligonucleotide fragment containing the desired sequence to be expressed. It is well known in the art that a methionine at the N-terminal position can be enzymatically cleaved by the use of the enzyme methionine aminopeptidase (MAP). MAP has been cloned from E. coli (Ben-Bassat et al. (1987) J. Bacteriol. 169:751-757) and Salmonella typhimurium and its in vitro activity has been demonstrated on recombinant proteins (Miller et al. (1987) PNAS 84:2718-1722). Therefore, removal of an N-terminal methionine, if desired, can be achieved either in vivo by expressing hedgehog-derived polypeptides in a host which produces MAP (e.g., E. coli or CM89 or S. cerevisiae), or in vitro by use of purified MAP (e.g., procedure of Miller et al., supra).

Alternatively, the coding sequences for the polypeptide can be incorporated as a part of a fusion gene including a nucleotide sequence encoding a different polypeptide. It is widely appreciated that fusion proteins can also facilitate the expression of proteins, and accordingly, can be used in the expression of the hedgehog polypeptides of the present invention. For example, hedgehog polypeptides can be generated as glutathione-S-transferase (GST-fusion) proteins. Such GST-fusion proteins can enable easy purification of the hedgehog polypeptide, as for example by the use of glutathione-derivatized matrices (see, for example, Current Protocols in Molecular Biology, eds. Ausubel et al. (N.Y.: John Wiley & Sons, 1991)). In another embodiment, a fusion gene coding for a purification leader sequence, such as a poly(His)/enterokinase cleavage site sequence, can be used to replace the signal sequence which naturally occurs at the N-terminus of the hedgehog protein (e.g. of the pro-form, in order to permit purification of the poly(His)-hedgehog protein by affinity chromatography using a Ni²⁺ metal resin. The purification leader sequence can then be subsequently removed by treatment with enterokinase (e.g., see Hochuli et al. (1987) J. Chromatography 411:177; and Janknecht et al. PNAS 88:8972).

Techniques for making fusion genes are known to those skilled in the art. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al. John Wiley & Sons: 1992).

Hedgehog polypeptides may also be chemically modified to create hedgehog derivatives by forming covalent or aggregate conjugates with other chemical moieties, such as glycosyl groups, cholesterol, isoprenoids, lipids, phosphate, acetyl groups and the like. Covalent derivatives of hedgehog proteins can be prepared by linking the chemical moieties to functional groups on amino acid sidechains of the protein or at the N-terminus or at the C-terminus of the polypeptide.

For instance, hedgehog proteins can be generated to include a moiety, other than sequence naturally associated with the protein, that binds a component of the extracellular matrix and enhances localization of the analog to cell surfaces. For example, sequences derived from the fibronectin “type-III repeat”, such as a tetrapeptide sequence R-G-D-S (Pierschbacher et al. (1984) Nature 309:30-3; and Komblihtt et al. (1985) EMBO 4:1755-9) can be added to the hedgehog polypeptide to support attachment of the chimeric molecule to a cell through binding ECM components (Ruoslahti et al. (1987) Science 238:491-497; Pierschbacheret al. (1987) J. Biol. Chem. 262:17294-8.; Hynes (1987) Cell 48:549-54; and Hynes (1992) Cell 69:11-25).

In a preferred embodiment, the hedgehog polypeptide is isolated from, or is otherwise substantially free of, other cellular proteins, especially other extracellular or cell surface associated proteins which may normally be associated with the hedgehog polypeptide, unless provided in the form of fusion protein with the hedgehog polypeptide. The term “substantially free of other cellular or extracellular proteins” (also referred to herein as “contaminating proteins”) or “substantially pure preparations” or “purified preparations” are defined as encompassing preparations of hedgehog polypeptides having less than 20% (by dry weight) contaminating protein, and preferably having less than 5% contaminating protein. By “purified”, it is meant that the indicated molecule is present in the substantial absence of other biological macromolecules, such as other proteins. The term “purified” as used herein preferably means at least 80% by dry weight, more preferably in the range of 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above.

As described above for recombinant polypeptides, isolated hedgehog polypeptides can include all or a portion of the amino acid sequences represented in any of SEQ ID Nos:10-18 or 20, or a homologous sequence thereto. Preferred fragments of the subject hedgehog proteins correspond to the N-terminal and C-terminal proteolytic fragments of the mature protein. Bioactive fragments of hedgehog polypeptides are described in great detail in PCT publications WO 95/18856 and WO 96/17924.

With respect to bioctive fragments of hedgehog polypeptide, preferred hedgehog therapeutics include at least 50 (contiguous) amino acid residues of a hedgehog polypeptide, more preferably at least 100 (contiguous), and even more preferably at least 150 (contiguous) residues.

Another preferred hedgehog polypeptide which can be included in the hedgehog therapeutic is an N-terminal fragment of the mature protein having a molecular weight of approximately 19 kDa.

Preferred human hedgehog proteins include N-terminal fragments corresponding approximately to residues 24-197 of SEQ ID No. 15, 28-202 of SEQ ID No. 16, and 23-198 of SEQ ID No. 17. By “corresponding approximately” it is meant that the sequence of interest is at most 20 amino acid residues different in length to the reference sequence, though more preferably at most 5, 10 or 15 amino acid different in length.

As described above for recombinant polypeptides, isolated hedgehog polypeptides can include all or a portion of the amino acid sequences represented in SEQ ID No:10, SEQ ID No:11, SEQ ID No:12, SEQ ID No:13, SEQ ID No:14, SEQ ID No:15, SEQ ID No:16, SEQ ID No:17, SEQ ID No:18 or SEQ ID No:20, or a homologous sequence thereto. Preferred fragments of the subject hedgehog proteins correspond to the N-terminal and C-terminal proteolytic fragments of the mature protein. Bioactive fragments of hedgehog polypeptides are described in great detail in PCT publications WO 95/18856 and WO 96/17924.

Still other preferred hedgehog polypeptides includes an amino acid sequence represented by the formula A-B wherein: (i) A represents all or the portion of the amino acid sequence designated by residues 1-168 of SEQ ID No:21; and B represents at least one amino acid residue of the amino acid sequence designated by residues 169-221 of SEQ ID No:21; (ii) A represents all or the portion of the amino acid sequence designated by residues 24-193 of SEQ ID No:15; and B represents at least one amino acid residue of the amino acid sequence designated by residues 194-250 of SEQ ID No:15; (iii) A represents all or the portion of the amino acid sequence designated by residues 25-193 of SEQ ID No:13; and B represents at least one amino acid residue of the amino acid sequence designated by residues 194-250 of SEQ ID No:13; (iv) A represents all or the portion of the amino acid sequence designated by residues 23-193 of SEQ ID No:11; and B represents at least one amino acid residue of the amino acid sequence designated by residues 194-250 of SEQ ID No:11; (v) A represents all or the portion of the amino acid sequence designated by residues 28-197 of SEQ ID No:12; and B represents at least one amino acid residue of the amino acid sequence designated by residues 198-250 of SEQ ID No:12; (vi) A represents all or the portion of the amino acid sequence designated by residues 29-197 of SEQ ID No:16; and B represents at least one amino acid residue of the amino acid sequence designated by residues 198-250 of SEQ ID No:16; or (vii) A represents all or the portion of the amino acid sequence designated by residues 23-193 of SEQ ID No. 17, and B represents at least one amino acid residue of the amino acid sequence designated by residues 194-250 of SEQ ID No. 17. In certain preferred embodiments, A and B together represent a contiguous polypeptide sequence designated sequence, A represents at least 25, 50, 75, 100, 125 or 150 (contiguous) amino acids of the designated sequence, and B represents at least 5, 10, or 20 (contiguous) amino acid residues of the amino acid sequence designated by corresponding entry in the sequence listing, and A and B together preferably represent a contiguous sequence corresponding to the sequence listing entry. Similar fragments from other hedgehog also contemplated, e.g., fragments which correspond to the preferred fragments from the sequence listing entries which are enumerated above. In preferred embodiments, the hedgehog polypeptide includes a C-terminal glycine (or other appropriate residue) which is derivatized with a cholesterol.

Isolated peptidyl portions of hedgehog proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, a hedgehog polypeptide of the present invention may be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of a wild-type (e.g., “authentic”) hedgehog protein. For example, Roman et al. (1994) Eur J Biochem 222:65-73 describe the use of competitive-binding assays using short, overlapping synthetic peptides from larger proteins to identify binding domains.

The recombinant hedgehog polypeptides of the present invention also include homologs of the authentic hedgehog proteins, such as versions of those protein which are resistant to proteolytic cleavage, as for example, due to mutations which alter potential cleavage sequences or which inactivate an enzymatic activity associated with the protein. Hedgehog homologs of the present invention also include proteins which have been post-translationally modified in a manner different than the authentic protein. Exemplary derivatives of hedgehog proteins include polypeptides which lack N-glycosylation sites (e.g. to produce an unglycosylated protein), which lack sites for cholesterolization, and/or which lack N-terminal and/or C-terminal sequences.

Modification of the structure of the subject hedgehog polypeptides can also be for such purposes as enhancing therapeutic or prophylactic efficacy, or stability (e.g., ex vivo shelf life and resistance to proteolytic degradation in vivo). Such modified peptides, when designed to retain at least one activity of the naturally-occurring form of the protein, are considered functional equivalents of the hedgehog polypeptides described in more detail herein. Such modified peptides can be produced, for instance, by amino acid substitution, deletion, or addition.

It is well known in the art that one could reasonably expect that certain isolated replacements of amino acids, e.g., replacement of an amino acid residue with another related amino acid (i.e. isosteric and/or isoelectric mutations), can be carried out without major effect on the biological activity of the resulting molecule. Conservative replacements are those that take place within a family of amino acids that are related in their side chains. Genetically encoded amino acids are can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan, and tyrosine are sometimes classified jointly as aromatic amino acids. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 2nd ed., Ed. by L. Stryer, WH Freeman and Co.: 1981). Whether a change in the amino acid sequence of a peptide results in a functional hedgehog homolog (e.g. functional in the sense that it acts to mimic or antagonize the wild-type form) can be readily determined by assessing the ability of the variant peptide to produce a response in cells in a fashion similar to the wild-type protein, or competitively inhibit such a response. Polypeptides in which more than one replacement has taken place can readily be tested in the same manner.

It is specifically contemplated that the methods of the present invention can be carried using homologs of naturally occurring hedgehog proteins. In one embodiment, the invention contemplates using hedgehog polypeptides generated by combinatorial mutagenesis. Such methods, as are known in the art, are convenient for generating both point and truncation mutants, and can be especially useful for identifying potential variant sequences (e.g. homologs) that are functional in binding to a receptor for hedgehog proteins. The purpose of screening such combinatorial libraries is to generate, for example, novel hedgehog homologs which can act as either agonists or antagonist. To illustrate, hedgehog homologs can be engineered by the present method to provide more efficient binding to a cognate receptor, such as patched, yet still retain at least a portion of an activity associated with hedgehog. Thus, combinatorially-derived homologs can be generated to have an increased potency relative to a naturally occurring form of the protein. Likewise, hedgehog homologs can be generated by the present combinatorial approach to act as antagonists, in that they are able to mimic, for example, binding to other extracellular matrix components (such as receptors), yet not induce any biological response, thereby inhibiting the action of authentic hedgehog or hedgehog agonists. Moreover, manipulation of certain domains of hedgehog by the present method can provide domains more suitable for use in fusion proteins, such as one that incorporates portions of other proteins which are derived from the extracellular matrix and/or which bind extracellular matrix components.

To further illustrate the state of the art of combinatorial mutagenesis, it is noted that the review article of Gallop et al. (1994) J Med Chem 37:1233 describes the general state of the art of combinatorial libraries as of the earlier 1990's. In particular, Gallop et al state at page 1239 “[s]creening the analog libraries aids in determining the minimum size of the active sequence and in identifying those residues critical for binding and intolerant of substitution”. In addition, the Ladner et al. PCT publication WO90/02809, the Goeddel et al. U.S. Pat. No. 5,223,408, and the Markland et al. PCT publication WO92/15679 illustrate specific techniques which one skilled in the art could utilize to generate libraries of hedgehog variants which can be rapidly screened to identify variants/fragments which retained a particular activity of the hedgehog polypeptides. These techniques are exemplary of the art and demonstrate that large libraries of related variants/truncants can be generated and assayed to isolate particular variants without undue experimentation. Gustin et al. (1993) Virology 193:653, and Bass et al. (1990) Proteins: Structure, Function and Genetics 8:309-314 also describe other exemplary techniques from the art which can be adapted as means for generating mutagenic variants of hedgehog polypeptides.

Indeed, it is plain from the combinatorial mutagenesis art that large scale mutagenesis of hedgehog proteins, without any preconceived ideas of which residues were critical to the biological function, and generate wide arrays of variants having equivalent biological activity. Indeed, it is the ability of combinatorial techniques to screen billions of different variants by high throughout analysis that removes any requirement of a priori understanding or knowledge of critical residues.

To illsutrate, the amino acid sequences for a population of hedgehog homologs or other related proteins are aligned, preferably to promote the highest homology possible. Such a population of variants can include, for example, hedgehog homologs from one or more species. Amino acids which appear at each position of the aligned sequences are selected to create a degenerate set of combinatorial sequences. In a preferred embodiment, the variegated library of hedgehog variants is generated by combinatorial mutagenesis at the nucleic acid level, and is encoded by a variegated gene library. For instance, a mixture of synthetic oligonucleotides can be enzymatically ligated into gene sequences such that the degenerate set of potential hedgehog sequences are expressible as individual polypeptides, or alternatively, as a set of larger fusion proteins (e.g. for phage display) containing the set of hedgehog sequences therein.

As illustrated in PCT publication WO 95/18856, to analyze the sequences of a population of variants, the amino acid sequences of interest can be aligned relative to sequence homology. The presence or absence of amino acids from an aligned sequence of a particular variant is relative to a chosen consensus length of a reference sequence, which can be real or artificial.

In an illustrative embodiment, alignment of exons 1, 2 and a portion of exon 3 encoded sequences (e.g. the N-terminal approximately 221 residues of the mature protein) of each of the Shh clones produces a degenerate set of Shh polypeptides represented by the general formula:

| | |
|---|---|
| C-G-P-G-R-G-X(1)-G-X(2)-R-R-H-P-K-K-L-T-P-L-A-Y-K-Q-F-I-P-N-V-A-E- | (SEQ ID No: 21 |
| K-T-L-G-A-S-G-R-Y-E-G-K-I-X(3)-R-N-S-E-R-F-K-E-L-T-P-N-Y-N-P-D-I-I-F- | |
| K-D-E-E-N-T-G-A-D-R-L-M-T-Q-R-C-K-D-K-L-N-X(4)-L-A-I-S-V-M-N-X(5)- | |
| W-P-G-V-X(6)-L-R-V-T-E-G-W-D-E-D-G-H-H-X(7)-E-E-S-L-H-Y-E-G-R-A- | |
| V-D-I-T-T-S-D-R-D-X(8)-S-K-Y-G-X(9)-L-X(10)-R-L-A-V-E-A-G-F-D-W-V- | |
| Y-Y-E-S-K-A-H-I-H-C-S-V-K-A-E-N-S-V-A-A-K-S-G-G-C-F-P-G-S-A-X(11)- | |
| V-X(12)-L-X(13)-X(14)-G-G-X(15)-K-X-(16)-V-K-D-L-X(17)-P-G-D-X(18)-V- | |
| L-A-A-D-X(19)-X(20)-G-X(21)-L-X(22)-X(23)-S-D-F-X(24)-X(25)-F-X(26)-D- | |
| R | |

wherein each of the degenerate positions “X” can be an amino acid which occurs in that position in one of the human, mouse, chicken or zebrafish Shh clones, or, to expand the library, each X can also be selected from amongst amino acid residue which would be conservative substitutions for the amino acids which appear naturally in each of those positions. For instance, Xaa(1) represents Gly, Ala, Val, Leu, Ile, Phe, Tyr or Trp ; Xaa(2) represents Arg, His or Lys; Xaa(3) represents Gly, Ala, Val, Leu, Ile, Ser or Thr; Xaa(4) represents Gly, Ala, Val, Leu, Ile, Ser or Thr; Xaa(5) represents Lys, Arg, His, Asn or Gln; Xaa(6) represents Lys, Arg or His; Xaa(7) represents Ser, Thr, Tyr, Trp or Phe; Xaa(8) represents Lys, Arg or His; Xaa(9) represents Met, Cys, Ser or Thr; Xaa(10) represents Gly, Ala, Val, Leu, Ile, Ser or Thr; Xaa(11) represents Leu, Val, Met, Thr or Ser; Xaa(12) represents His, Phe, Tyr, Ser, Thr, Met or Cys; Xaa(13) represents Gln, Asn, Glu, or Asp; Xaa(14) represents His, Phe, Tyr, Thr, Gln, Asn, Glu or Asp; Xaa(15) represents Gln, Asn, Glu, Asp, Thr, Ser, Met or Cys; Xaa(16) represents Ala, Gly, Cys, Leu, Val or Met; Xaa(17) represents Arg, Lys, Met, Ile, Asn, Asp, Glu, Gln, Ser, Thr or Cys; Xaa(18) represents Arg, Lys, Met or Ile; Xaa(19) represents Ala, Gly, Cys, Asp, Glu, Gln, Asn, Ser, Thr or Met; Xaa(20) represents Ala, Gly, Cys, Asp, Asn, Glu or Gln; Xaa(21) represents Arg, Lys, Met, Ile, Asn, Asp, Glu or Gln; Xaa(22) represent Leu, Val, Met or Ile; Xaa(23) represents Phe, Tyr, Thr, His or Trp; Xaa(24) represents Ile, Val, Leu or Met; Xaa(25) represents Met, Cys, Ile, Leu, Val, Thr or Ser; Xaa(26) represents Leu, Val, Met, Thr or Ser. In an even more expansive library, each X can be selected from any amino acid.

In similar fashion, alignment of each of the human, mouse, chicken and zebrafish hedgehog clones, can provide a degenerate polypeptide sequence represented by the general formula:

| | |
|---|---|
| C-G-P-G-R-G-X(1)-X(2)-X(3)-R-R-X(4)-X(5)-X(6)-P-K-X(7)-L-X(8)-P-L-X(9)- | (SEQ ID No: 22 |
| Y-K-Q-F-X(10)-P-X(11)-X(12)-X(13)-E-X(14)-T-L-G-A-S-G-X(15)-X(16)-E-G- | |
| X(17)-X(18)-X(19)-R-X(20)-S-E-R-F-X(21)-X(22)-L-T-P-N-Y-N-P-D-I-I-F-K- | |
| D-E-E-N-X(23)-G-A-D-R-L-M-T-X(24)-R-C-K-X(25)-X(26)-X(27)-N-X(28)-L- | |
| A-I-S-V-M-N-X(29)-W-P-G-V-X(30)-L-R-V-T-E-G-X(31)-D-E-D-G-H-H- | |
| X(32)-X(33)-X(34)-S-L-H-Y-E-G-R-A-X(35)-D-I-T-T-S-D-R-D-X(36)-X(37)- | |
| K-Y-G-X(38)-L-X(39)-R-L-A-V-E-A-G-F-D-W-V-Y-Y-E-S-X(40)-X(41)-H- | |
| X(42)-H-X(43)-S-V-K-X(44)-X(45) | |

wherein, as above, each of the degenerate positions “X” can be an amino acid which occurs in a corresponding position in one of the wild-type clones, and may also include amino acid residue which would be conservative substitutions, or each X can be any amino acid residue. In an exemplary embodiment, Xaa(1) represents Gly, Ala, Val, Leu, Ile, Pro, Phe or Tyr; Xaa(2) represents Gly, Ala, Val, Leu or Ile; Xaa(3) represents Gly, Ala, Val, Leu, Ile, Lys, His or Arg; Xaa(4) represents Lys, Arg or His; Xaa(5) represents Phe, Trp, Tyr or an amino acid gap; Xaa(6) represents Gly, Ala, Val, Leu, Ile or an amino acid gap; Xaa(7) represents Asn, Gln, His, Arg or Lys; Xaa(8) represents Gly, Ala, Val, Leu, Ile, Ser or Thr; Xaa(9) represents Gly, Ala, Val, Leu, Ile, Ser or Thr; Xaa(10) represents Gly, Ala, Val, Leu, Ile, Ser or Thr; Xaa(11) represents Ser, Thr, Gln or Asn; Xaa(12) represents Met, Cys, Gly, Ala, Val, Leu, Ile, Ser or Thr; Xaa(13) represents Gly, Ala, Val, Leu, Ile or Pro; Xaa(14) represents Arg, His or Lys; Xaa(15) represents Gly, Ala, Val, Leu, Ile, Pro, Arg, His or Lys; Xaa(16) represents Gly, Ala, Val, Leu, Ile, Phe or Tyr; Xaa(17) represents Arg, His or Lys; Xaa(18) represents Gly, Ala, Val, Leu, Ile, Ser or Thr; Xaa(19) represents Thr or Ser; Xaa(20) represents Gly, Ala, Val, Leu, Ile, Asn or Gln; Xaa(21) represents Arg, His or Lys; Xaa(22) represents Asp or Glu; Xaa(23) represents Ser or Thr; Xaa(24) represents Glu, Asp, Gln or Asn; Xaa(25) represents Glu or Asp; Xaa(26) represents Arg, His or Lys; Xaa(27) represents Gly, Ala, Val, Leu or Ile; Xaa(28) represents Gly, Ala, Val, Leu, Ile, Thr or Ser; Xaa(29) represents Met, Cys, Gln, Asn, Arg, Lys or His; Xaa(30) represents Arg, His or Lys; Xaa(31) represents Trp, Phe, Tyr, Arg, His or Lys; Xaa(32) represents Gly, Ala, Val, Leu, Ile, Ser, Thr, Tyr or Phe; Xaa(33) represents Gln, Asn, Asp or Glu; Xaa(34) represents Asp or Glu; Xaa(35) represents Gly, Ala, Val, Leu, or Ile; Xaa(36) represents Arg, His or Lys; Xaa(37) represents Asn, Gln, Thr or Ser; Xaa(38) represents Gly, Ala, Val, Leu, Ile, Ser, Thr, Met or Cys; Xaa(39) represents Gly, Ala, Val, Leu, Ile, Thr or Ser; Xaa(40) represents Arg, His or Lys; Xaa(41) represents Asn, Gln, Gly, Ala, Val, Leu or Ile; Xaa(42) represents Gly, Ala, Val, Leu or Ile; Xaa(43) represents Gly, Ala, Val, Leu, Ile, Ser, Thr or Cys; Xaa(44) represents Gly, Ala, Val, Leu, Ile, Thr or Ser; and Xaa(45) represents Asp or Glu.

There are many ways by which the library of potential hedgehog homologs can be generated from a degenerate oligonucleotide sequence. Chemical synthesis of a degenerate gene sequence can be carried out in an automatic DNA synthesizer, and the synthetic genes then ligated into an appropriate expression vector. The purpose of a degenerate set of genes is to provide, in one mixture, all of the sequences encoding the desired set of potential hedgehog sequences. The synthesis of degenerate oligonucleotides is well known in the art (see for example, Narang, SA (1983) Tetrahedron 39:3; Itakura et al. (1981) Recombinant DNA, Proc 3rd Cleveland Sympos. Macromolecules, ed. A G Walton, Amsterdam: Elsevier pp273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

A wide range of techniques are known in the art for screening gene products of combinatorial libraries made by point mutations, and for screening cDNA libraries for gene products having a certain property. Such techniques will be generally adaptable for rapid screening of the gene libraries generated by the combinatorial mutagenesis of hedgehog homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate hedgehog sequences created by combinatorial mutagenesis techniques.

In one embodiment, the combinatorial library is designed to be secreted (e.g. the polypeptides of the library all include a signal sequence but no transmembrane or cytoplasmic domains), and is used to transfect a eukaryotic cell that can be co-cultured with pre-adipocyte (stem or progenitor) cells. A functional hedgehog protein secreted by the cells expressing the combinatorial library will diffuse to neighboring pre-adipocyte cells and induce a particular biological response, such as proliferation or differentiation. The pattern of detection of such a change in phenotype will resemble a gradient function, and will allow the isolation (generally after several repetitive rounds of selection) of cells producing hedgehog homologs active as anti-adipocytic agents. Likewise, hedgehog antagonists can be selected in similar fashion by the ability of the cell producing a functional antagonist to protect neighboring cells (e.g., to inhibit proliferation) from the effect of wild-type hedgehog added to the culture media.

To illustrate, target pre-adipocyte cells are cultured in 24-well microtitre plates. Other eukaryotic cells are transfected with the combinatorial hedgehog gene library and cultured in cell culture inserts (e.g. Collaborative Biomedical Products, Catalog #40446) that are able to fit into the wells of the microtitre plate. The cell culture inserts are placed in the wells such that recombinant hedgehog homologs secreted by the cells in the insert can diffuse through the porous bottom of the insert and contact the target cells in the microtitre plate wells. After a period of time sufficient for functional forms of a hedgehog protein to produce a measurable response in the target cells, such as growth state, the inserts are removed and the effect of the variant hedgehog proteins on the target cells determined. Cells from the inserts corresponding to wells which score positive for activity can be split and re-cultured on several inserts, the process being repeated until the active clones are identified.

In yet another screening assay, the candidate hedgehog gene products are displayed on the surface of a cell or viral particle, and the ability of particular cells or viral particles to associate with a hedgehog-binding moiety (such as the patched protein or other hedgehog receptor) via this gene product is detected in a “panning assay”. Such panning steps can be carried out on cells cultured from embryos. For instance, the gene library can be cloned into the gene for a surface membrane protein of a bacterial cell, and the resulting fusion protein detected by panning (Ladner et al., WO 88/06630; Fuchs et al. (1991) Bio/Technology 9:1370-1371; and Goward et al. (1992) TIBS 18:136-140). In a similar fashion, fluorescently labeled molecules which bind hedgehog can be used to score for potentially functional hedgehog homologs. Cells can be visually inspected and separated under a fluorescence microscope, or, where the morphology of the cell permits, separated by a fluorescence-activated cell sorter.

In an alternate embodiment, the gene library is expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at very high concentrations, large number of phage can be screened at one time. Second, since each infectious phage displays the combinatorial gene product on its surface, if a particular phage is recovered from an affinity matrix in low yield, the phage can be amplified by another round of infection. The group of almost identical E.coli filamentous phages M13, fd, and fl are most often used in phage display libraries, as either of the phage gIII or gVIII coat proteins can be used to generate fusion proteins without disrupting the ultimate packaging of the viral particle (Ladner et al. PCT publication WO 90/02909; Garrard et al., PCT publication WO 92/09690; Marks et al. (1992) J. Biol. Chem. 267:16007-16010; Griffths et al. (1993) EMBO J 12:725-734; Clackson et al. (1991) Nature 352:624-628; and Barbas et al. (1992) PNAS 89:4457-4461).

In an illustrative embodiment, the recombinant phage antibody system (RPAS, Pharamacia Catalog number 27-9400-01) can be easily modified for use in expressing and screening hedgehog combinatorial libraries. For instance, the pCANTAB 5 phagemid of the RPAS kit contains the gene which encodes the phage gIII coat protein. The hedgehog combinatorial gene library can be cloned into the phagemid adjacent to the gIII signal sequence such that it will be expressed as a gIII fusion protein. After ligation, the phagemid is used to transform competent E. coli TG1 cells. Transformed cells are subsequently infected with M13KO7 helper phage to rescue the phagemid and its candidate hedgehog gene insert. The resulting recombinant phage contain phagemid DNA encoding a specific candidate hedgehog, and display one or more copies of the corresponding fusion coat protein. The phage-displayed candidate hedgehog proteins which are capable of binding an hedgehog receptor are selected or enriched by panning. For instance, the phage library can be applied to cells which express the patched protein and unbound phage washed away from the cells. The bound phage is then isolated, and if the recombinant phage express at least one copy of the wild type gIII coat protein, they will retain their ability to infect E. coli. Thus, successive rounds of reinfection of E. coli, and panning will greatly enrich for hedgehog homologs, which can then be screened for further biological activities in order to differentiate agonists and antagonists.

Combinatorial mutagenesis has a potential to generate very large libraries of mutant proteins, e.g., in the order of 10²⁶ molecules. Combinatorial libraries of this size may be technically challenging to screen even with high throughput screening assays such as phage display. To overcome this problem, a new technique has been developed recently, recursive ensemble mutagenesis (REM), which allows one to avoid the very high proportion of non-functional proteins in a random library and simply enhances the frequency of functional proteins, thus decreasing the complexity required to achieve a useful sampling of sequence space. REM is an algorithm which enhances the frequency of functional mutants in a library when an appropriate selection or screening method is employed (Arkin and Yourvan, 1992, PNAS USA 89:7811-7815; Yourvan et al., 1992, Parallel Problem Solving from Nature, 2., In Maenner and Manderick, eds., Elsevir Publishing Co., Amsterdam, pp. 401-410; Delgrave et al., 1993, Protein Engineering 6(3):327-331).

The invention also provides for reduction of the hedgehog protein to generate mimetics, e.g. peptide or non-peptide agents, which are able to disrupt binding of a hedgehog polypeptide of the present invention with an hedgehog receptor. Thus, such mutagenic techniques as described above are also useful to map the determinants of the hedgehog proteins which participate in protein-protein interactions involved in, for example, binding of the subject hedgehog polypeptide to other extracellular matrix components. To illustrate, the critical residues of a subject hedgehog polypeptide which are involved in molecular recognition of an hedgehog receptor such as patched can be determined and used to generate hedgehog-derived peptidomimetics which competitively inhibit binding of the authentic hedgehog protein with that moiety. By employing, for example, scanning mutagenesis to map the amino acid residues of each of the subject hedgehog proteins which are involved in binding other extracellular proteins, peptidomimetic compounds can be generated which mimic those residues of the hedgehog protein which facilitate the interaction. Such mimetics may then be used to interfere with the normal function of a hedgehog protein. For instance, non-hydrolyzable peptide analogs of such residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffinan et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), -turn dipeptide cores (Nagai et al. (1985) Tetrahedron Lett 26:647; and Sato et al. (1986) J Chem Soc Perkin Trans 1:1231), and -aminoalcohols (Gordon et al. (1985) Biochem Biophys Res Commun 126:419; and Dann et al. (1986) Biochem Biophys Res Commun 134:71).

Recombinantly produced forms of the hedgehog proteins can be produced using, e.g, expression vectors containing a nucleic acid encoding a hedgehog polypeptide, operably linked to at least one transcriptional regulatory sequence. Operably linked is intended to mean that the nucleotide sequence is linked to a regulatory sequence in a manner which allows expression of the nucleotide sequence. Regulatory sequences are art-recognized and are selected to direct expression of a hedgehog polypeptide. Accordingly, the term transcriptional regulatory sequence includes promoters, enhancers and other expression control elements. Such regulatory sequences are described in Goeddel; Gene Expression Technology: Methods in Enzymology 185, Academic Press, San Diego, Calif. (1990). For instance, any of a wide variety of expression control sequences, sequences that control the expression of a DNA sequence when operatively linked to it, may be used in these vectors to express DNA sequences encoding hedgehog polypeptide. Such useful expression control sequences, include, for example, a viral LTR, such as the LTR of the Moloney murine leukemia virus, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage , the control regions for fd coat protein, the promoter for 3-phosphoglycerate kinase or other glycolytic enzymes, the promoters of acid phosphatase, e.g., Pho5, the promoters of the yeast-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other proteins encoded by the vector, such as antibiotic markers, should also be considered.

In addition to providing a ready source of hedgehog polypeptides for purification, the gene constructs of the present invention can also be used as a part of a gene therapy protocol to deliver nucleic acids encoding either an agonistic or antagonistic form of a hedgehog polypeptide. Thus, another aspect of the invention features expression vectors for in vivo transfection of a hedgehog polypeptide in particular cell types so as cause ectopic expression of a hedgehog polypeptide in an adipocyte tissue.

Formulations of such expression constructs may be administered in any biologically effective carrier, e.g. any formulation or composition capable of effectively delivering the recombinant gene to cells in vivo. Approaches include insertion of the hedgehog coding sequence in viral vectors including recombinant retroviruses, adenovirus, adeno-associated virus, and herpes simplex virus-1, or recombinant bacterial or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO₄ precipitation carried out in vivo. It will be appreciated that because transduction of appropriate target cells represents the critical first step in gene therapy, choice of the particular gene delivery system will depend on such factors as the phenotype of the intended target and the route of administration, e.g. locally or systemically. Furthermore, it will be recognized that the particular gene construct provided for in vivo transduction of hedgehog expression are also useful for in vitro transduction of cells, such as for use in the ex vivo tissue culture systems described below.

A preferred approach for in vivo introduction of nucleic acid into a cell is by use of a viral vector containing nucleic acid, e.g. a cDNA, encoding the particular form of the hedgehog polypeptide desired. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors are generally understood to be the recombinant gene delivery system of choice for the transfer of exogenous genes in vivo, particularly into humans. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) has been replaced by nucleic acid encoding a hedgehog polypeptide and renders the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines for preparing both ecotropic and amphotropic retroviral systems include Crip, Cre, 2 and Am. Retroviruses have been used to introduce a variety of genes into many different cell types, including adipocyte cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Furthermore, it has been shown that it is possible to limit the infection spectrum of retroviruses and consequently of retroviral-based vectors, by modifying the viral packaging proteins on the surface of the viral particle (see, for example PCT publications WO93/25234 and WO94/06920). For instance, strategies for the modification of the infection spectrum of retroviral vectors include: coupling antibodies specific for cell surface antigens to the viral env protein (Roux et al. (1989) PNAS 86:9079-9083; Julan et al. (1992) J. Gen Virol 73:3251-3255; and Goud et al. (1983) Virology 163:251-254); or coupling cell surface receptor ligands to the viral env proteins (Neda et al. (1991) J Biol Chem 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, can also be used to convert an ecotropic vector in to an amphotropic vector.

Moreover, use of retroviral gene delivery can be further enhanced by the use of tissue- or cell-specific transcriptional regulatory sequences which control expression of the hedgehog gene of the retroviral vector.

Another viral gene delivery system useful in the present method utilizes adenovirus-derived vectors. The genome of an adenovirus can be manipulated such that it encodes and expresses a gene product of interest but is inactivated in terms of its ability to replicate in a normal lytic viral life cycle. See for example Berkner et al. (1988) Bio Techniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses can be advantageous in certain circumstances in that they can be used to infect a wide variety of cell types, including adipocyte cells. Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, e.g., Jones et al. (1979) Cell 6:683; Berkner et al., supra; and Graham et al. in Methods in Molecular Biology, E. J. Murray, Ed. (Humana, Clifton, N.J., 1991) vol. 7. pp. 109-127). Expression of the inserted hedgehog gene can be under control of, for example, the E1A promoter, the major late promoter (MLP) and associated leader sequences, the E3 promoter, or exogenously added promoter sequences.

In addition to viral transfer methods, such as those illustrated above, non-viral methods can also be employed to cause expression of a hedgehog polypeptide in the tissue of an animal. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the hedgehog polypeptide gene by the targeted cell. Exemplary gene delivery systems of this type include liposomal derived systems, poly-lysine conjugates, and artificial viral envelopes.

In clinical settings, the gene delivery systems for the therapeutic hedgehog gene can be introduced into a patient by any of a number of methods, each of which is familiar in the art. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al. (1994) PNAS 91: 3054-3057). A hedgehog expression construct can be delivered in a gene therapy construct to dermal cells by, e.g., electroporation using techniques described, for example, by Dev et al. ((1994) Cancer Treat Rev 20:105-115).

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced intact from recombinant cells, e.g. retroviral vectors, the pharmaceutical preparation can comprise one or more cells which produce the gene delivery system.

In yet another embodiment, the hedgehog or ptc therapeutic can be a “gene activation” construct which, by homologous recombination with a genomic DNA, alters the transcriptional regulatory sequences of an endogenous gene. For instance, the gene activation construct can replace the endogenous promoter of a hedgehog gene with a heterologous promoter, e.g., one which causes consitutive expression of the hedgehog gene or which causes inducible expression of the gene under conditions different from the normal expression pattern of the gene. Other genes in the patched signaling pathway can be similarly targeted. A vareity of different formats for the gene activation constructs are available. See, for example, the Transkaryotic Therapies, Inc PCT publications WO93/09222, WO95/31560, WO96/29411, WO95/31560 and WO94/12650.

In preferred embodiments, the nucleotide sequence used as the gene activation construct can be comprised of (1) DNA from some portion of the endogenous hedgehog gene (exon sequence, intron sequence, promoter sequences, etc.) which direct recombination and (2) heterologous transcriptional regulatory sequence(s) which is to be operably linked to the coding sequence for the genomic hedgehog gene upon recombination of the gene activation construct. For use in generating cultures of hedgehog producing cells, the construct may further include a reporter gene to detect the presence of the knockout construct in the cell.

The gene activation construct is inserted into a cell, and integrates with the genomic DNA of the cell in such a position so as to provide the heterologous regulatory sequences in operative association with the native hedgehog gene. Such insertion occurs by homologous recombination, i.e., recombination regions of the activation construct that are homologous to the endogenous hedgehog gene sequence hybridize to the genomic DNA and recombine with the genomic sequences so that the construct is incorporated into the corresponding position of the genomic DNA.

The terms “recombination region” or “targeting sequence” refer to a segment (i.e., a portion) of a gene activation construct having a sequence that is substantially identical to or substantially complementary to a genomic gene sequence, e.g., including 5′ flanking sequences of the genomic gene, and can facilitate homologous recombination between the genomic sequence and the targeting transgene construct.

As used herein, the term “replacement region” refers to a portion of a activation construct which becomes integrated into an endogenous chromosomal location following homologous recombination between a recombination region and a genomic sequence.

The heterologous regulatory sequences, e.g., which are provided in the replacement region, can include one or more of a variety elements, including: promoters (such as constitutive or inducible promoters), enhancers, negative regualtory elements, locus control regions, transcription factor binding sites, or combinations thereof. Promoters/enhancers which may be used to control the expression of the targeted gene in vivo include, but are not limited to, the cytomegalovirus (CMV) promoter/enhancer (Karasuyama et al., 1989, J. Exp. Med., 169:13), the human β-actin promoter (Gunning et al. (1987) PNAS 84:4831-4835), the glucocorticoid-inducible promoter present in the mouse mammary tumor virus long terminal repeat (MMTV LTR) (Kiessig et al. (1984) Mol Cell Biol. 4:1354-1362), the long terminal repeat sequences of Moloney murine leukemia virus (MuLV LTR) (Weiss et al. (1985) RNA Tumor Viruses, Cold Spring Harbor Laboratory, Cold Spring Harbor, N.Y.), the SV40 early or late region promoter (Bemoist et al. (1981) Nature 290:304-310; Templeton et al. (1984) Mol Cell Biol., 4:817; and Sprague et al. (1983) J. Virol., 45:773), the promoter contained in the 3′ long terminal repeat of Rous sarcoma virus (RSV) (Yamamoto et al., 1980, Cell, 22:787-797), the herpes simplex virus (HSV) thymidine kinase promoter/enhancer (Wagner et al. (1981) PNAS 82:3567-71), and the herpes simplex virus LAT promoter (Wolfe et al. (1992) Nature Genetics, 1:379-384).

In an exemplary embodiment, portions of the 5′ flanking region of the human Shh gene are amplified using primers which add restriction sites, to generate the following fragments

5′-gcgcgcttcgaaGCGAGGCAGCCAGCGAGGGAGAGAGCGAGCGGGCGAGCCGGAGC-
GAGGAAatcgatgcgcgc (primer 1)
5′-gcgcgcagatctGGGAAAGCGCAAGAGAGAGCGCACACGCACACACCCGCCGCGCG-
CACTCGggatccgcgcgc (primer 2)

As illustrated, primer 1 includes a 5′ non-coding region of the human Shh gene and is flanked by an AsuII and ClaI restriction sites. Primer 2 includes a portion of the 5′ non-coding region immediately 3′ to that present in primer 1. The hedgehog gene sequence is flanked by XhoII and BamHI restriction sites. The purified amplimers are cut with each of the enzymes as appropriate.

The vector pCDNA1.1 (Invitrogen) includes a CMV promoter. The plasmid is cut with with AsuII, which cleaves just 3′ to the CMV promoter sequence. The AsuII/ClaI fragment of primer 1 is ligated to the AsuII cleavage site of the pcDNA vector. The ClaI/AsuII ligation destroys the AsuII site at the 3′ end of a properly inserted primer 1.

The vector is then cut with BamHI, and an XhoII/BamHI fragment of primer 2 is ligated to the BamHI cleavage site. As above, the BamHI/XhoII ligation destroys the BamHI site at the 5′ end of a properly inserted primer 2.

Individual colonies are selected, cut with AsuII and BamHI, and the size of the AsuII/BamHI fragment determined. Colonies in which both the primer 1 and primer 2 sequences are correctly inserted are further amplified, an cut with AsuII and BamHI to produce the gene activation construct

cgaagcgaggcagccagcgagggagagagcgagcgggcgagccggagcgaggaaATCGAAGGTTCGAATCCTTC
CCCCACCACCATCACTTTCAAAAGTCCGAAAGAATCTGCTCCCTGCTTGTGTGTTGG
AGGTCGCTGAGTAGTGCGCGAGTAAAATTTAAGCTACAACAAGGCAAGGCTTGACC
GACAATTGCATGAAGAATCTGCTTAGGGTTAGGCGTTTTGCGCTGCTTCGCGATGTA
CGGGCCAGATATACGCGTTGACATTGATTATTGACTAGTTATTAATAGTAATCAATT
ACGGGGTCATTAGTTCATAGCCCATATATGGAGTTCCGCGTTACATAACTTACGGTA
AATGGCCCGCCTGGCTGACCGCCCAACGACCCCCGCCCATTGACGTCAATAATGAC
GTATGTTCCCATAGTAACGCCAATAGGGACTTTCCATTGACGTCAATGGGTGGACTA
TTTACGGTAAACTGCCCACTTGGCAGTACATCAAGTGTATCATATGCCAAGTACGCC
CCCTATTGACGTCAATGACGGTAAATGGCCCGCCTGGCATTATGCCCAGTACATGAC
CTTATGGGACTTTCCTACTTGGCAGTACATCTACGTATTAGTCATCGCTATTACCATG
GTGATGCGGTTTTGGCAGTACATCAATGGGCGTGGATAGCGGTTTGACTCACGGGGA
TTTCCAAGTCTCCACCCCATTGACGTCAATGGGAGTTTGTTTTGGCACCAAAATCAA
CGGGACTTTCCAAAATGTCGTAACAACTCCGCCCCATTGACGCAAATGGGCGGTAG
GCGTGTACGGTGGGAGGTCTATATAAGCAGAGCTCTCTGGCTAACTAGAGAACCCA
CTGCTTACTGGCTTATCGAAATTAATACGACTCACTATAGGGAGACCCAAGCTTGGT
ACCGAGCTCGGATCgatctgggaaagcgcaagagagagcgcacacgcacacacccgccgcgcgcactcgg

In this construct, the flanking primer 1 and primer 2 sequences provide the recombination region which permits the insertion of the CMV promoter in front of the coding sequence for the human Shh gene. Other heterologous promoters (or other transcriptional regulatory sequences) can be inserted in a genomic hedgehog gene by a similar method.

In still other embodiments, the replacement region merely deletes a negative transcriptional control element of the native gene, e.g., to activate expression, or ablates a positive control element, e.g., to inhibit expression of the targeted gene.

### V. Exemplary ptc Therapeutic Compounds

In another embodiment, the subject method is carried out using a ptc therapeutic composition. Such compositions can be generated with, for example, compounds which bind to patched and alter its signal transduction activity, compounds which alter the binding and/or enzymatic activity of a protein (e.g., intracellular) involved in patched signal pathway, and compounds which alter the level of expression of a hedgehog protein, a patched protein or a protein involved in the intracellular signal transduction pathway of patched.

The availability of purified and recombinant hedgehog polypeptides facilitates the generation of assay systems which can be used to screen for drugs, such as small organic molecules, which are either agonists or antagonists of the normal cellular function of a hedgehog and/or patched protein, particularly their role in the pathogenesis of adipocyte cell proliferation and/or differentiation. In one embodiment, the assay evaluates the ability of a compound to modulate binding between a hedgehog polypeptide and a hedgehog receptor such as patched. In other embodiments, the assay merely scores for the ability of a test compound to alter the signal transduction acitity of the patched protein. In this manner, a variety of hedgehog and/or ptc therapeutics, both proliferative and anti-proliferative in activity, can be identified. A variety of assay formats will suffice and, in light of the present disclosure, will be comprehended by skilled artisan.

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with receptor proteins.

Acordingly, in an exemplary screening assay for ptc therapeutics, the compound of interest is contacted with a mixture including a hedgehog receptor protein (e.g., a cell expressing the patched receptor) and a hedgehog protein under conditions in which it is ordinarily capable of binding the hedgehog protein. To the mixture is then added a composition containing a test compound. Detection and quantification of receptor/hedgehog complexes provides a means for determining the test compound's efficacy at inhibiting (or potentiating) complex formation between the receptor protein and the hedgehog polypeptide. The efficacy of the compound can be assessed by generating dose response curves from data obtained using various concentrations of the test compound. Moreover, a control assay can also be performed to provide a baseline for comparison. In the control assay, isolated and purified hedgehog polypeptide is added to the receptor protein, and the formation of receptor/hedgehog complex is quantitated in the absence of the test compound.

In other embodiments, a ptc therapeutic of the present invention is one which disrupts the association of patched with smoothened.

Agonist and antagonists of adipocyte cell growth can be distinguished, and the efficacy of the compound can be assessed, by subsequent testing with pre- and adipocyte cells, e.g., in culture.

In an illustrative embodiment, the polypeptide utilized as a hedgehog receptor can be generated from the patched protein. Accordingly, an exemplary screening assay includes all or a suitable portion of the patched protein which can be obtained from, for example, the human patched gene (GenBank U43148) or other vertebrate sources (see GenBank Accession numbers U40074 for chicken patched and U46155 for mouse patched), as well as from drosophila (GenBank Accession number M28999) or other invertebrate sources. The patched protein can be provided in the screening assay as a whole protein (preferably expressed on the surface of a cell), or alternatively as a fragment of the full length protein which binds to hedgehog polypeptides, e.g., as one or both of the substantial extracellular domains (e.g. corresponding to residues Asn120-Ser438 and/or Arg770-Trp1027 of the human patched protein—which are also potential antagonists of hedgehog-dependent signal transduction). For instance, the patched protein can be provided in soluble form, as for example a preparation of one of the extracellular domains, or a preparation of both of the extracellular domains which are covalently connected by an unstructured linker (see, for example, Huston et al. (1988) PNAS 85:4879; and U.S. Pat. No. 5,091,513). In other embodiments, the protein can be provided as part of a liposomal preparation or expressed on the surface of a cell. The patched protein can derived from a recombinant gene, e.g., being ectopically expressed in a heterologous cell. For instance, the protein can be expressed on oocytes, mammalian cells (e.g., COS, CHO, 3T3 or the like), or yeast cell by standard recombinant DNA techniques. These recombinant cells can be used for receptor binding, signal transduction or gene expression assays. Marigo et al. (1996) Development 122:1225-1233 illustrates a binding assay of human hedgehog to chick patched protein ectopically expressed in Xenopus laevis oocytes. The assay system of Marigo et al. can be adapted to the present drug screening assays. As illustrated in that reference, Shh binds to the patched protein in a selective, saturable, dose-dependent manner, thus demonstrating that patched is a receptor for Shh.

Complex formation between the hedgehog polypeptide and a hedgehog receptor may be detected by a variety of techniques. For instance, modulation of the formation of complexes can be quantitated using, for example, detectably labelled proteins such as radiolabelled, fluorescently labelled, or enzymatically labelled hedgehog polypeptides, by immunoassay, or by chromatographic detection.

Typically, for cell-free assays, it will be desirable to immobilize either the hedgehog receptor or the hedgehog polypeptide to facilitate separation of receptor/hedgehog complexes from uncomplexed forms of one of the proteins, as well as to accommodate automation of the assay. In one embodiment, a fusion protein can be provided which adds a domain that allows the protein to be bound to a matrix. For example, glutathione-S-transferase/receptor (GST/receptor) fusion proteins can be adsorbed onto glutathione sepharose beads (Sigma Chemical, St. Louis, Mo.) or glutathione derivatized microtitre plates, which are then combined with the hedgehog polypeptide, e.g. an ³⁵S-labeled hedgehog polypeptide, and the test compound and incubated under conditions conducive to complex formation, e.g. at physiological conditions for salt and pH, though slightly more stringent conditions may be desired. Following incubation, the beads are washed to remove any unbound hedgehog polypeptide, and the matrix bead-bound radiolabel determined directly (e.g. beads placed in scintillant), or in the supernatant after the receptor/hedgehog complexes are dissociated. Alternatively, the complexes can be dissociated from the bead, separated by SDS-PAGE gel, and the level of hedgehog polypeptide found in the bead fraction quantitated from the gel using standard electrophoretic techniques.

Other techniques for immobilizing proteins on matrices are also available for use in the subject assay. For instance, soluble portions of the hedgehog receptor protein can be immobilized utilizing conjugation of biotin and streptavidin. For instance, biotinylated receptor molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with the hedgehog receptor but which do not interfere with hedgehog binding can be derivatized to the wells of the plate, and the receptor trapped in the wells by antibody conjugation. As above, preparations of a hedgehog polypeptide and a test compound are incubated in the receptor-presenting wells of the plate, and the amount of receptor/hedgehog complex trapped in the well can be quantitated. Exemplary methods for detecting such complexes, in addition to those described above for the GST-immobilized complexes, include immunodetection of complexes using antibodies reactive with the hedgehog polypeptide, or which are reactive with the receptor protein and compete for binding with the hedgehog polypeptide; as well as enzyme-linked assays which rely on detecting an enzymatic activity associated with the hedgehog polypeptide. In the instance of the latter, the enzyme can be chemically conjugated or provided as a fusion protein with the hedgehog polypeptide. To illustrate, the hedgehog polypeptide can be chemically cross-linked or genetically fused with alkaline phosphatase, and the amount of hedgehog polypeptide trapped in the complex can be assessed with a chromogenic substrate of the enzyme, e.g. paranitrophenylphosphate. Likewise, a fusion protein comprising the hedgehog polypeptide and glutathione-S-transferase can be provided, and complex formation quantitated by detecting the GST activity using 1-chloro-2,4-dinitrobenzene (Habig et al (1974) J Biol Chem 249:7130).

For processes which rely on immunodetection for quantitating one of the proteins trapped in the complex, antibodies against the protein, such as the anti-hedgehog antibodies described herein, can be used. Alternatively, the protein to be detected in the complex can be “epitope tagged” in the form of a fusion protein which includes, in addition to the hedgehog polypeptide or hedgehog receptor sequence, a second polypeptide for which antibodies are readily available (e.g. from commercial sources). For instance, the GST fusion proteins described above can also be used for quantification of binding using antibodies against the GST moiety. Other useful epitope tags include myc-epitopes (e.g., see Ellison et al. (1991) J Biol Chem 266:21150-21157) which includes a 10-residue sequence from c-myc, as well as the pFLAG system (International Biotechnologies, Inc.) or the pEZZ-protein A system (Pharamacia, N.J.).

Where the desired portion of the hedgehog receptor (or other hedgehog binding molecule) cannot be provided in soluble form, liposomal vesicles can be used to provide manipulatable and isolatable sources of the receptor. For example, both authentic and recombinant forms of the patched protein can be reconstituted in artificial lipid vesicles (e.g. phosphatidylcholine liposomes) or in cell membrane-derived vesicles (see, for example, Bear et al. (1992) Cell 68:809-818; Newton et al. (1983) Biochemistry 22:6110-6117; and Reber et al. (1987) J Biol Chem 262:11369-11374).

In addition to cell-free assays, such as described above, the readily available source of hedgehog proteins provided by the art also facilitates the generation of cell-based assays for identifying small molecule agonists/antagonists and the like. Analogous to the cell-based assays described above for screening combinatorial libraries, cells which are sensitive to hedgehog induction, e.g. patched-expressing cells or other adipocyte-derived cells sensitive to hedgehog induction, can be contacted with a hedgehog protein and a test agent of interest, with the assay scoring for anything from simple binding to the cell to modulation in hedgehog inductive responses by the target cell in the presence and absence of the test agent. As with the cell-free assays, agents which produce a statistically significant change in hedgehog activities (either inhibition or potentiation) can be identified.

In other emdodiments, the cell-based assay scores for agents which disrupt association of patched and smoothened proteins, e.g., in the cell surface membrane or liposomal preparation.

In addition to characterizing cells that naturally express the patched protein, cells which have been genetically engineered to ectopically express patched can be utilized for drug screening assays. As an example, cells which either express low levels or lack expression of the patched protein, e.g. Xenopus laevis oocytes, COS cells or yeast cells, can be genetically modified using standard techniques to ectopically express the patched protein. (see Marigo et al., supra).

The resulting recombinant cells, e.g., which express a functional patched receptor, can be utilized in receptor binding assays to identify agonist or anatagonsts of hedgehog binding. Binding assays can be performed using whole cells. Furthermore, the recombinant cells of the present invention can be engineered to include other heterolgous genes encoding proteins involved in hedgehog-dependent siganl pathways. For example, the gene products of one or more of smoothened, costal-2 and/or fused can be co-expressed with patched in the reagent cell, with assays being sensitive to the functional reconstituion of the hedgehog signal transduction cascade.

Alternatively, liposomal preparations using reconstituted patched protein can be utilized. Patched protein purified from detergent extracts from both authentic and recombinant origins can be reconstituted in in artificial lipid vesicles (e.g. phosphatidylcholine liposomes) or in cell membrane-derived vesicles (see, for example, Bear et al. (1992) Cell 68:809-818; Newton et al. (1983) Biochemistry 22:6110-6117; and Reber et al. (1987) J Biol Chem 262:11369-11374). The lamellar structure and size of the resulting liposomes can be characterized using electron microscopy. External orientation of the patched protein in the reconstituted membranes can be demonstrated, for example, by immunoelectron microscopy. The hedgehog protein binding activity of liposomes containing patched and liposomes without the protein in the presence of candidate agents can be compared in order to identify potential modulators of the hedgehog-patched interaction.

The hedgehog protein used in these cell-based assays can be provided as a purified source (natural or recombinant in origin), or in the form of cells/tissue which express the protein and which are co-cultured with the target cells. As in the cell-free assays, where simple binding (rather than induction) is the hedgehog activity scored for in the assay, the protein can be labelled by any of the above-mentioned techniques, e.g., fluorescently, enzymatically or radioactively, or detected by immunoassay.

In addition to binding studies, functional assays can be used to identified modulators, i.e., agonists or antagonists, of hedgehog or patched activities. By detecting changes in intracellular signals, such as alterations in second messengers or gene expression, in patched-expressing cells contacted with a test agent, candidate agonists and antagonists to patched signaling can be identified.

A number of gene products have been implicated in patched-mediated signal transduction, including patched, the transcription factor cubitus interruptus (ci), the serine/threonine kinasefused (fu) and the gene products of costal-2, smoothened and suppressor of fused.

The interaction of a hedgehog protein with patched sets in motion a cascade involving the activation and inhibition of downstream effectors, the ultimate consequence of which is, in some instances, a detectable change in the transcription or translation of a gene. Potential transcriptional targets of patched signaling are the patched gene itself (Hidalgo and Ingham, 1990 Development 110, 291-301; Marigo et al., 1996) and the vertebrate homologs of the drosophila cubitus interruptus gene, the GLI genes (Hui et al. (1994) Dev Biol 162:402-413). Patched gene expression has been shown to be induced in cells of the limb bud and the neural plate that are responsive to Shh. (Marigo et al. (1996) PNAS, in press; Marigo et al. (1996) Development 122:1225-1233). The GLI genes encode putative transcription factors having zinc finger DNA binding domains (Orenic et al. (1990) Genes & Dev 4:1053-1067; Kinzler et al. (1990) Mol Cell Biol 10:634-642). Transcription of the GLI gene has been reported to be upregulated in response to hedgehog in limb buds, while transcription of the GLI3 gene is downregulated in response to hedgehog induction (Marigo et al. (1996) Development 122:1225-1233). By selecting transcriptional regulatory sequences from such target genes, e.g. from patched or GLI genes, that are responsible for the up- or down regulation of these genes in response to patched signalling, and operatively linking such promoters to a reporter gene, one can derive a transcription based assay which is sensitive to the ability of a specific test compound to modify patched signalling pathways. Expression of the reporter gene, thus, provides a valuable screening tool for the development of compounds that act as agonists or antagonists of ptc induction of differentiation/quiescence.

Reporter gene based assays of this invention measure the end stage of the above described cascade of events, e.g., transcriptional modulation. Accordingly, in practicing one embodiment of the assay, a reporter gene construct is inserted into the reagent cell in order to generate a detection signal dependent on ptc signaling. To identify potential regulatory elements responsive to ptc signaling present in the transcriptional regulatory sequence of a target gene, nested deletions of genomic clones of the target gene can be constructed using standard techniques. See, for example, Current Protocols in Molecular Biology, Ausubel, F. M. et al. (eds.) Greene Publishing Associates, (1989); U.S. Pat. No. 5,266,488; Sato et al. (1995) J Biol Chem 270:10314-10322; and Kube et al. (1995) Cytokine 7:1-7. A nested set of DNA fragments from the gene's 5′-flanking region are placed upstream of a reporter gene, such as the luciferase gene, and assayed for their ability to direct reporter gene expression in patched expressing cells. Host cells transiently transfected with reporter gene constructs can be scored for the induction of expression of the reporter gene in the presence and absence of hedgehog to determine regulatory sequences which are responsice to patched-dependent signalling.

In practicing one embodiment of the assay, a reporter gene construct is inserted into the reagent cell in order to generate a detection signal dependent on second messengers generated by induction with hedgehog protein. Typically, the reporter gene construct will include a reporter gene in operative linkage with one or more transcriptional regulatory elements responsive to the hedgehog activity, with the level of expression of the reporter gene providing the hedgehog-dependent detection signal. The amount of transcription from the reporter gene may be measured using any method known to those of skill in the art to be suitable. For example, mRNA expression from the reporter gene may be detected using RNAse protection or RNA-based PCR, or the protein product of the reporter gene may be identified by a characteristic stain or an intrinsic activity. The amount of expression from the reporter gene is then compared to the amount of expression in either the same cell in the absence of the test compound (or hedgehog) or it may be compared with the amount of transcription in a substantially identical cell that lacks the target receptor protein. Any statistically or otherwise significant difference in the amount of transcription indicates that the test compound has in some manner altered the signal transduction of the patched protein, e.g., the test compound is a potential ptc therapeutic.

As described in further detail below, in preferred embodiments the gene product of the reporter is detected by an intrinsic activity associated with that product. For instance, the reporter gene may encode a gene product that, by enzymatic activity, gives rise to a detection signal based on color, fluorescence, or luminescence. In other preferred embodiments, the reporter or marker gene provides a selective growth advantage, e.g., the reporter gene may enhance cell viability, relieve a cell nutritional requirement, and/or provide resistance to a drug.

Preferred reporter genes are those that are readily detectable. The reporter gene may also be included in the construct in the form of a fusion gene with a gene that includes desired transcriptional regulatory sequences or exhibits other desirable properties. Examples of reporter genes include, but are not limited to CAT (chloramphenicol acetyl transferase) (Alton and Vapnek (1979), Nature 282: 864-869) luciferase, and other enzyme detection systems, such as beta-galactosidase; firefly luciferase (deWet et al. (1987), Mol. Cell. Biol. 7:725-737); bacterial luciferase (Engebrecht and Silverman (1984), PNAS 1: 4154-4158; Baldwin et al. (1984), Biochemistry 23: 3663-3667); alkaline phosphatase (Toh et al. (1989) Eur. J. Biochem. 182: 231-238, Hall et al. (1983) J. Mol. Appl. Gen. 2: 101), human placental secreted alkaline phosphatase (Cullen and Malim (1992) Methods in Enzymol. 216:362-368).

Transcriptional control elements which may be included in a reporter gene construct include, but are not limited to, promoters, enhancers, and repressor and activator binding sites. Suitable transcriptional regulatory elements may be derived from the transcriptional regulatory regions of genes whose expression is induced after modulation of a patched signal transduction pathway. The characteristics of preferred genes from which the transcriptional control elements are derived include, but are not limited to, low or undetectable expression in quiescent cells, rapid induction at the transcriptional level within minutes of extracellular simulation, induction that is transient and independent of new protein synthesis, subsequent shut-off of transcription requires new protein synthesis, and mRNAs transcribed from these genes have a short half-life. It is not necessary for all of these properties to be present.

In yet other embodiments, second messenger generation can be measured directly in the detection step, such as mobilization of intracellular calcium, phospholipid metabolism or adenylate cyclase activity are quantitated, for instance, the products of phospholipid hydrolysis IP₃, DAG or cAMP could be measured For example, recent studies have implicated protein kinase A (PKA) as a possible component of hedgehog/patched signaling (Hammerschmidt et al. (1996) Genes & Dev 10:647). High PKA activity has been shown to antagonize hedgehog signaling in these systems. Although it is unclear whether PKA acts directly downstream or in parallel with hedgehog signaling, it is possible that hedgehog signalling occurs via inhibition of PKA activity. Thus, detection of PKA activity provides a potential readout for the instant assays.

In a preferred embodiment, the ptc therapeutic is a PKA inhibitor. A variety of PKA inhibitors are known in the art, including both peptidyl and organic compounds. For instance, the ptc therapeutic can be a 5-isoquinolinesulfonamide, such as represented in the general formula:

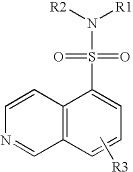

wherein,

R₁ and R₂ each can independently represent hydrogen, and as valence and stability permit a lower alkyl, a lower alkenyl, a lower alkynyl, a carbonyl (such as a carboxyl, an ester, a formate, or a ketone), a thiocarbonyl (such as a thioester, a thioacetate, or a thioformate), an amino, an acylamino, an amido, a cyano, a nitro, an azido, a sulfate, a sulfonate, a sulfonamido, —(CH₂)ₘ—R₈, —(CH₂)ₘ—OH, —(CH₂)ₘ—O-lower alkyl, —(CH₂)ₘ—O-lower alkenyl, —(CH₂)ₙ—O—(CH₂)ₘ—R₈, —(CH₂)ₘ—SH, —(CH₂)ₘ—S-lower alkyl, —(CH₂)ₘ—S-lower alkenyl, —(CH₂)ₙ—S—(CH₂)ₘ—R₈, or

R₁ and R₂ taken together with N form a heterocycle (substituted or unsubstituted);

R₃ is absent or represents one or more substitutions to the isoquinoline ring such as a lower alkyl, a lower alkenyl, a lower alkynyl, a carbonyl (such as a carboxyl, an ester, a formate, or a ketone), a thiocarbonyl (such as a thioester, a thioacetate, or a thioformate), an amino, an acylamino, an amido, a cyano, a nitro, an azido, a sulfate, a sulfonate, a sulfonamido, —(CH₂)ₘ—R₈, —(CH₂)ₘ—OH, —(CH₂)ₘ—O-lower alkyl, —(CH₂)ₘ—O-lower alkenyl, —(CH₂)ₙ—O—(CH₂)ₘ—R₈, —(CH₂)ₘ—SH, —(CH₂)ₘ—S-lower alkyl, —(CH₂)ₘ—S-lower alkenyl, —(CH₂)ₙ—S—(CH₂)ₘ—R₈;

R₈ represents a substituted or unsubstituted aryl, aralkyl, cycloalkyl, cycloalkenyl, or heterocycle; and

n and m are independently for each occurrence zero or an integer in the range of 1 to 6.

In a preferred embodiment, the PKA inhibitor is N-[2-((p-bromocinnamyl)amino)ethyl]-5-isoquinolinesulfonamide (H-89; Calbiochem Cat. No. 371963), e.g., having the formula:

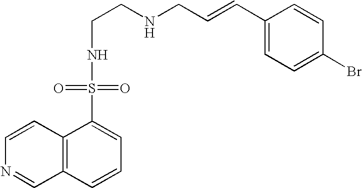

In another embodiment, the PKA inhibitor is 1-(5-isoquinolinesulfonyl)-2-methylpiperazine (H-7; Calbiochem Cat. No. 371955), e.g., having the formula:

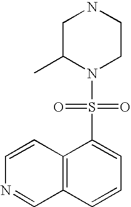

In still other embodiments, the PKA inhibitor is KT5720 (Calbiochem Cat. No. 420315), having the structure

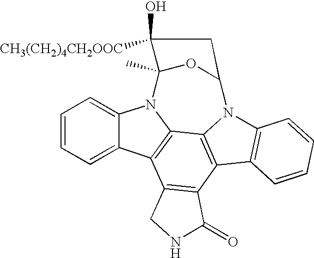

A variety of nucleoside analogs are also useful as PKA inhibitors. For example, the subject method can be carried out cyclic AMP analogs which inhibit the kinase activity of PKA, as for example, 8-bromo-cAMP or dibutyryl-cAMP

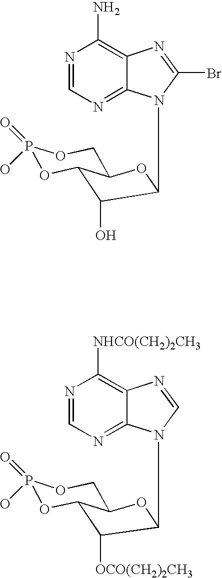

Exemplary peptidyl inhibitors of PKA activity include the PKA Heat Stable Inhibitor (isoform; see, for example, Calbiochem Cat. No. 539488, and Wen et al. (1995) J Biol Chem 270:2041).

Certain hedehog receptors may stimulate the activity of phospholipases. Inositol lipids can be extracted and analyzed using standard lipid extraction techniques. Water soluble derivatives of all three inositol lipids (IP₁, IP2, IP₃) can also be quantitated using radiolabelling techniques or HPLC.

The mobilization of intracellular calcium or the influx of calcium from outside the cell may be a response to hedgehog stimulation or lack there of. Calcium flux in the reagent cell can be measured using standard techniques. The choice of the appropriate calcium indicator, fluorescent, bioluminescent, metallochromic, or Ca⁺⁺-sensitive microelectrodes depends on the cell type and the magnitude and time constant of the event under study (Borle (1990) Environ Health Perspect 84:45-56). As an exemplary method of Ca⁺⁺ detection, cells could be loaded with the Ca⁺⁺ sensitive fluorescent dye fura-2 or indo-1, using standard methods, and any change in Ca⁺⁺ measured using a fluorometer.

In certain embodiments of the assay, it may be desirable to screen for changes in cellular phosphorylation. As an example, the drosophila gene fused (fu) which encodes a senne/threonine kinase has been identified as a potential downstream target in hedgehog signaling. (Preat et al., 1990 Nature 347, 87-89; Therond et al. 1993, Mech. Dev. 44. 65-80). The ability of compounds to modulate serine/threonine kinase activation could be screened using colony immunoblotting (Lyons and Nelson (1984) Proc. Natl. Acad. Sci. USA 81:7426-7430) using antibodies against phosphorylated serine or threonine residues. Reagents for performing such assays are commercially available, for example, phosphoserine and phosphothreonine specific antibodies which measure increases in phosphorylation of those residues can be purchased from comercial sources.

In yet another embodiment, the ptc therapeutic is an antisense molecule which inhibits expression of a protein involved in a patched-mediated signal transduction pathway. To illustrate, by inhibiting the expression of a protein which are involved in patched signals, such as fused, costal-2, smoothened and/or Gli genes, the ability of the patched signal pathway(s) to inhibit proliferation of a cell can be altered, e.g., potentiated or repressed.

As used herein, “antisense” therapy refers to administration or in situ generation of oligonucleotide probes or their derivatives which specifically hybridize (e.g. bind) under cellular conditions with cellular mRNA and/or genomic DNA encoding a hedgehog protein, patched, or a protein involved in patched-mediated signal transduction. The hybridization should inhibit expression of that protein, e.g. by inhibiting transcription and/or translation. The binding may be by conventional base pair complementarity, or, for example, in the case of binding to DNA duplexes, through specific interactions in the major groove of the double helix. In general, “antisense” therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the target cellular mRNA. Alternatively, the antisense construct is an oligonucleotide probe which is generated ex vivo and which, when introduced into the cell causes inhibition of expression by hybridizing with the mRNA and/or genomic sequences of a target gene. Such oligonucleotide probes are preferably modified oligonucleotide which are resistant to endogenous nucleases, e.g. exonucleases and/or endonucleases, and is therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) Biotechniques 6:958-976; and Stein et al. (1988) Cancer Res 48:2659-2668.

Several considerations should be taken into account when constructing antisense oligonucleotides for the use in the methods of the invention: (1) oligos should have a GC content of 50% or more; (2) avoid sequences with stretches of 3 or more G's; and (3) oligonucleotides should not be longer than 25-26 mers. When testing an antisense oligonucleotide, a mismatched control can be constructed. The controls can be generated by reversing the sequence order of the corresponding antisense oligonucleotide in order to conserve the same ratio of bases.

In an illustrative embodiment, the ptc therapeutic can be an antisense construct for inhibiting the expression of patched, e.g., to mimic the inhibition of patched by hedgehog. Exemplary antisense constructs include:

5′-GTCCTGGCGCCGCCGCCGCCGTCGCC
5′-TTCCGATGACCGGCCTTTCGCGGTGA
5′-GTGCACGGAAAGGTGCAGGCCACACT

### VI. Exemplary Pharmaceutical Preparations of Hedgehog and ptc Therapeutics

The source of the hedgehog and ptc therapeutics to be formulated will depend on the particular form of the agent. Small organic molecules and peptidyl fragments can be chemically synthesized and provided in a pure form suitable for pharmaceutical/cosmetic usage. Products of natural extracts can be purified according to techniques known in the art. For example, the Cox et al. U.S. Pat. No. 5,286,654 describes a method for purifying naturally occurring forms of a secreted protein and can be adapted for purification of hedgehog polypeptides. Recombinant sources of hedgehog polypeptides are also available. For example, the gene encoding hedgehog polypeptides, are known, inter alia, from PCT publications WO 95/18856 and WO 96/17924.

Those of skill in treating adipocyte tissues can determine the effective amount of an hedgehog or ptc therapeutic to be formulated in a pharmaceutical or cosmetic preparation.

The hedgehog or ptc therapeutic formulations used in the method of the invention are most preferably applied in the form of appropriate compositions. As appropriate compositions there may be cited all compositions usually employed for systemically or topically administering drugs. The pharmaceutically acceptable carrier should be substantially inert, so as not to act with the active component. Suitable inert carriers include water, alcohol polyethylene glycol, mineral oil or petroleum gel, propylene glycol and the like.

To prepare the pharmaceutical compositions of this invention, an effective amount of the particular hedgehog or ptc therapeutic as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, particularly, for administration orally, rectally, percutaneously, or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules, and tablets. Because of their ease in administration, tablets and capsules represents the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations which are intended to be converted, shortly before use, to liquid form preparations. In the compositons suitable for percutaneous administration, the carrier optionally comprises a penetration enhancing agent and/or a suitable wetting agent, optionally combined with suitable additives of any nature in minor proportions, which additives do not introduce a significant deleterious effect on the skin.

In addition to the direct topical application of the preparations they can be topically administered by other methods, for example, encapsulated in a temperature and/or pressure sensitive matrix or in film or solid carrier which is soluble in body fluids and the like for subsequent release, preferably sustained-release of the active component.

As appropriate compositions for topical application there may be cited all compositions usually employed for topically administering therapeuitcs, e.g., creams, gellies, dressings, shampoos, tinctures, pastes, ointments, salves, powders, liquid or semiliquid formulation and the like. Application of said compositions may be by aerosol e.g. with a propellent such as nitrogen carbon dioxide, a freon, or without a propellent such as a pump spray, drops, lotions, or a semisolid such as a thickened composition which can be applied by a swab. In particular compositions, semisolid compositions such as salves, creams, pastes, gellies, ointments and the like will conveniently be used.

It is especially advantageous to formulate the subject compositions in dosage unit form for ease of administration and uniformity of dosage. Dosage unit form as used in the specification and claims herein refers to physically discreate units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powders packets, wafers, injectable solutions or suspensions, teaspoonfuls, tablespoonfuls and the like, and segregated multiples thereof.

The pharmaceutical preparations of the present invention can be used, as stated above, for the many applications whcih can be considered cosmetic uses. Cosmetic compositions known in the art, preferably hypoallergic and pH controlled are especially preferred, and include toilet waters, packs, lotions, skin milks or milky lotions. The preparations contain, besides the hedgehog or ptc therapeutic, components usually employed in such preparations. Examples of such components are oils, fats, waxes, surfactants, humectants, thickening agents, antioxidants, viscosity stabilizers, chelating agents, buffers, preservatives, perfumes, dyestuffs, lower alkanols, and the like. If desired, further ingredients may be incorporated in the compositions, e.g. antiinflammatory agents, antibacterials, antifungals, disinfectants, vitamins, sunscreens, antibiotics, or other anti-acne agents.

Examples of oils comprise fats and oils such as olive oil and hydrogenated oils; waxes such as beeswax and lanolin; hydrocarbons such as liquid paraffin, ceresin, and squalane; fatty acids such as stearic acid and oleic acid; alcohols such as cetyl alcohol, stearyl alcohol, lanolin alcohol, and hexadecanol; and esters such as isopropyl myristate, isopropyl palmitate and butyl stearate. As examples of surfactants there may be cited anionic surfactants such as sodium stearate, sodium cetylsulfate, polyoxyethylene laurylether phosphate, sodium N-acyl glutamate; cationic surfactants such as stearyldimethylbenzylammonium chloride and stearyltrimethylammonium chloride; ampholytic surfactants such as alkylaminoethylglycine hydrocloride solutions and lecithin; and nonionic surfactants such as glycerin monostearate, sorbitan monostearate, sucrose fatty acid esters, propylene glycol monostearate, polyoxyethylene oleylether, polyethylene glycol monostearate, polyoxyethylene sorbitan monopalmitate, polyoxyethylene coconut fatty acid monoethanolamide, polyoxypropylene glycol (e.g. the materials sold under the trademark “Pluronic”), polyoxyethylene castor oil, and polyoxyethylene lanolin. Examples of humectants include glycerin, 1,3-butylene glycol, and propylene glycol; examples of lower alcohols include ethanol and isopropanol; examples of thickening agents include xanthan gum, hydroxypropyl cellulose, hydroxypropyl methyl cellulose, polyethylene glycol and sodium carboxymethyl cellulose; examples of antioxidants comprise butylated hydroxytoluene, butylated hydroxyanisole, propyl gallate, citric acid and ethoxyquin; examples of chelating agents include disodium edetate and ethanehydroxy diphosphate; examples of buffers comprise citric acid, sodium citrate, boric acid, borax, and disodium hydrogen phosphate; and examples of preservatives are methyl parahydroxybenzoate, ethyl parahydroxybenzoate, dehydroacetic acid, salicylic acid and benzoic acid.

For preparing ointments, creams, toilet waters, skin milks, and the like, typically from 0.01 to 10% in particular from 0.1 to 5% and more in particular from 0.2 to 2.5% of the active ingredient, e.g., of the hedgehog or ptc therapeutic, will be incorporated in the compositions. In ointments or creams, the carrier for example consists of 1 to 20%, in particular 5 to 15% of a humectant, 0.1 to 10% in particular from 0.5 to 5% of a thickener and water; or said carrier may consist of 70 to 99%, in particular 20 to 95% of a surfactant, and 0 to 20%, in particular 2.5 to 15% of a fat; or 80 to 99.9% in particular 90 to 99% of a thickener; or 5 to 15% of a surfactant, 2-15% of a humectant, 0 to 80% of an oil, very small (<2%) amounts of preservative, coloring agent and/or perfume, and water. In a toilet water, the carrier for example consists of 2 to 10% of a lower alcohol, 0.1 to 10% or in particular 0.5 to 1% of a surfactant, 1 to 20%, in particular 3 to 7% of a humectant, 0 to 5% of a buffer, water and small amounts (<2%) of preservative, dyestuff and/or perfume. In a skin milk, the carrier typically consists of 10-50% of oil, 1 to 10% of surfactant, 50-80% of water and 0 to 3% of preservative and/or perfume. In the aforementioned preparations, all % symbols refer to weight by weight percentage.

Particular compositions for use in the method of the present invention are those wherein the hedgehog or ptc therapeutic is formulated in liposome-containing compositions. Liposomes are artificial vesicles formed by amphiphatic molecules such as polar lipids, for example, phosphatidyl cholines, ethanolamines and serines, sphingomyelins, cardiolipins, plasmalogens, phosphatidic acids and cerebiosides. Liposomes are formed when suitable amphiphathic molecules are allowed to swell in water or aqueous solutions to form liquid crystals usually of multilayer structure comprised of many bilayers separated from each other by aqueous material (also referred to as coarse liposomes). Another type of liposome known to be consisting of a single bilayer encapsulating aqueous material is referred to as a unilamellar vesicle. If water-soluble materials are included in the aqueous phase during the swelling of the lipids they become entrapped in the aqueous layer between the lipid bilayers.

Water-soluble active ingredients such as, for example, various salt forms of a hedgehog polypeptide, are encapsulated in the aqueous spaces between the molecular layers. The lipid soluble active ingredient of hedgehog or ptc therapeutic, such as an organic mimetic, is predominantly incorporated into the lipid layers, although polar head groups may protude from the layer into the aqueous space. The encapsulation of these compounds can be achieved by a number of methods. The method most commonly used involves casting a thin film of phospholipid onto the walls of a flask by evaporation from an organic solvent. When this film is dispersed in a suitable aqueous medium, multilamellar liposomes are formed. Upon suitable sonication, the coarse liposomes form smaller similarly closed vesicles.

Water-soluble active ingredients are usually incorporated by dispersing the cast film with an aqueous solution of the compound. The unencapsulated compound is then removed by centrifugation, chromatography, dialysis or other art-known suitable procedures. The lipid-soluble active ingredient is usually incorporated by dissolving it in the organic solvent with the phospholipid prior to casting the film. If the solubility of the material in the lipid phase is not exceeded or the amount present is not in excess of that which can be bound to the lipid, liposomes prepared by the above method usually contain most of the material bound in the lipid bilayers; separation of the liposomes from unencapsulated material is not required.

A particularly convenient method for preparing liposome formulated forms of hedgehog and ptc therapeutics is the method described in EP-A-253,619, incorporated herein by reference. In this method, single bilayered liposomes containing encapsulated active ingredients are prepared by dissolving the lipid component in an organic medium, injecting the organic solution of the lipid component under pressure into an aqueous component while simultaneously mixing the organic and aqueous components with a high speed homogenizer or mixing means, whereupon the liposomes are formed spontaneously.

The single bilayered liposomes containing the encapsulated hedgehog or ptc therapeutic can be employed directly or they can be employed in a suitable pharmaceutically acceptable carrier for topical administration. The viscosity of the liposomes can be increased by the addition of one or more suitable thickening agents such as, for example xanthan gum, hydroxypropyl cellulose, hydroxypropyl methylcellulose and mixtures thereof. The aqueous component may consist of water alone or it may contain electrolytes, buffered systems and other ingredients, such as, for example, preservatives. Suitable electrolytes which can be employed include metal salts such as alkali metal and alkaline earth metal salts. The preferred metal salts are calcium chloride, sodium chloride and potassium chloride. The concentration of the electrolyte may vary from zero to 260 mM, preferably from 5 mM to 160 mM. The aqueous component is placed in a suitable vessel which can be adapted to effect homogenization by effecting great turbulence during the injection of the organic component. Homogenization of the two components can be accomplished within the vessel, or, alternatively, the aqueous and organic components may be injected separately into a mixing means which is located outside the vessel. In the latter case, the liposomes are formed in the mixing means and then transferred to another vessel for collection purpose.

The organic component consists of a suitable non-toxic, pharmaceutically acceptable solvent such as, for example ethanol, glycerol, propylene glycol and polyethylene glycol, and a suitable phospholipid which is soluble in the solvent. Suitable phospholipids which can be employed include lecithin, phosphatidylcholine, phosphatydylserine, phosphatidylethanolamine, phosphatidylinositol, lysophosphatidyl-choline and phospha-tidyl glycerol, for example. Other lipophilic additives may be employed in order to selectively modify the characteristics of the liposomes. Examples of such other additives include stearylamine, phosphatidic acid, tocopherol, cholesterol and lanolin extracts.

In addition, other ingredients which can prevent oxidation of the phospholipids may be added to the organic component. Examples of such other ingredients include tocopherol, butylated hydroxyanisole, butylated hydroxytoluene, ascorbyl palmitate and ascorbyl oleate. Preservatives such a benzoic acid, methyl paraben and propyl paraben may also be added.

Apart from the above-described compositions, use may be made of covers, e.g. plasters, bandages, dressings, gauze pads and the like, containing an appropriate amount of a hedgehog or ptc therapeutic. In some cases use may be made of plasters, bandages, dressings, gauze pads and the like which have been impregnated with a topical formulation containing the therapeutic formulation.

### Exemplification

The invention now being generally described, it will be more readily understood by reference to the following examples which are included merely for purposes of illustration of certain aspects and embodiments of the present invention, which are not intended to limit the invention.

All enzymes, media and reagents were obtained Roche Molecular Biochemicals (Mannheim, Germany) except where noted differently.

Cell Culture

Mouse fibroblast C3H10T1/2 cells (ATCC CCL 226) were grown as monolayers in DMEM with 10% fetal calf serum (Cansera, Greiner GmbH, Frickenhausen, Germany) at 37° C. in a 5% CO₂ atmosphere. Treatment with BMP-2, Shh or a combination of both was done for 72 h under serumfree conditions or for 11 days in the presence of 10% FCS. The medium with inducers was changed twice a week.

BMP-2 was provided by W. Sebald, Würzburg, Germany, and used at a concentration of 500 ng/ml. Human sonic hedgehog protein was derived from baculovirus-mediated expression in insect cells and applied to cells as described previously (Zehentner et al., 1999) The baculovirus supernatants containing approximately 20 μg/ml hedgehog protein were diluted 1:40 (v/v) in the assay. For RT-PCR the experiment was stopped with lysis buffer (4.5 M guanidin hydrochloride, 50 mM Tris-HCl, 30% TritonX-100 (w/v), pH 6.6) after 72 h. The cell lysate was stored at −80° C. until RNA extraction.

Oil Red O Staining

The stock solution of Oil Red O was prepared by dissolving 4.2 g of Oil Red O (Sigma-Aldrich Chemie GmbH, Deisenhofen, Germany) in 1200 ml isopropanol. The solution was left overnight at room temperature without stirring followed by filtration. At last 900 ml of water was added and solution was left overnight at 4° C. with stirring.

Cultures were fixed for 5 h with 4% paraformaldehyde in isotonic phosphate buffer. Cells were then stained for 2 h at 4° C. by complete immersion in the working solution of Oil Red O followed by two times rinsing with water. The lipid droplets in the adipocyte-like cells were observed under a microscope with 10-fold magnification.

RNA Isolation

RNA was isolated using the High Pure RNA Isolation Kit (Roche Molecular Biochemicals Cat. No. 1 828 665). RNA quantity was determined by measuring absorption at 260 nm, and quality was checked in an agarose gel.

Quantitative RT-PCR

PPAR-γ, aP2, gli, ptc and actin mRNAs were quantitated via competitive RT-PCR using a multigene standard (Gilliland et al., 1990). The following primers for standard and target amplification were used.

| | |
|---|---|
| PPARγ-F 5′ aat gga aga cca ctc cca c; | PPARγ-R 5′ tgg aga tgc agg ctc cac; |
| actin-F 5′ aca cct tct aca atg agc tgc g; | actin-R 5′ cgc tcg gtg agg atc ttc atg; |
| aP2-F 5′ atg tgt gat gct ttt gta gg; | aP2-R 5′ ttt tcc atc cca ttt ctg c; |
| gli-F 5′ ctg ata ctc tgg gat atg gg; | gli-R 5′ tca agt cga gga cac tgg ct; |
| ptc-F 5′ gca ttg gca gga gga gtt gat tg; | ptc-R 5′ ggg tcg tgg ttg tga agg gaa gc. |

After linearization the standard vector was transcribed with T7 RNA polymerase into standard cRNA and treated with DNase I. 0.5 μg cellular RNA and different concentrations of standard cRNA were combined and reverse transcribed. PCR was performed using Expand™ High Fidelity PCR System in a Perkin Elmer GeneAmp 9600 thermocycler. The polymerase chain reaction conditions were one cycle: 94° C. 3 min; 58° C. 1 min; 72° C. 2 min followed by 45 cycles of: 94° C. 20 sec; 58° C. 20 sec; 72° C. 1 min and completed with 5 min 72° C. PCR propducts were analyzed by gel electrophoresis with ethidium bromide staining.

In order to quantitate the PCR products resulting from competitive RT-PCR the intensity of the bands in the gel was measured with the gel imaging system E.A.S.Y. (Herolab GmbH, Wiesloch, Germany). The relative expression level of the target gene was calculated as follows: [(standard intensity, control)×(target intensity, induced)]/[(standard intensity, induced)×(target intensity control)]. The relative expression level of actin was used to normalize the target level in each sample. Relative expression levels of four independent RNA isolations were used for means ±SD.

Results

C3H10T1/2 cells were analyzed at molecular and morphological level after treatment with Shh or BMP-2 or a combination thereof. The expression profiles of adipocyte marker genes, aP2 and PPAR-γ, were monitored by quantitative RT-PCR, as well as the expression of the hedgehog response genes gli and patched (FIG. 1). The results of three independent measurements, normalized with actin, were used to calculate the relative expression level, shown in FIG. 1. The control sample has an expression level of one corresponding to the baseline. The expression changes are demonstrated in the agarose gel of one measurement by comparing the intensity of the PCR fragments resulting from cellular mRNAs to the internal standard.

The expression of the two adipocytic marker genes was significantly upregulated by BMP-2 (sample 1.1 and 2.1 of FIG. 1A and B). After 72 h of serumfree culture (samples 1.1) PPAR-γ expression was increased 3.5-fold and aP2 expression 3-fold by BMP-2 in comparison to untreated cells. After 11 days of treatment (samples 2.1) PPAR-γ expression was still increased 2.5-fold and aP2 expression 2.1-fold.

In contrast to BMP-2, no upregulation of these mRNAs was detected after the treatment for 72 h or 11 days with Sonic hedgehog (samples 1.2 and 2.2). Instead, expression of the two adipocytic marker genes was downregulated by Shh to 25% of the control level in the 11 d culture (samples 2.2).

Combined treatment with BMP-2 and Shh caused adipocyte specific gene expression at control levels in the 72 h experiment (sample 1.3 of A and B) and a decrease in expression down to 50% of the control level after 11 d (sample 2.3 of A and B).

In addition to quantification of adipocyte marker genes by competitive RT-PCR, the expression of hedgehog responsive genes was analyzed. BMP-2 increased expression of the transcription factor gli 3-fold, but only during the 72 h treatment in serumfree conditions (FIG. 1C, sample 1.1). After 11 d BMP-2 in the presence of serum had no effect on the expession of gli (1C, sample 2.1). The mRNA of gli was significantly upregulated (6-fold) by Shh at both time points (FIG. 1C, samples 1.2 and 2.2). The combination of BMP-2 and Shh increased gli expression 10-fold after 72 h in serumfree media (1C, sample 1.3) and more than 7-fold after 11 days compared to the untreated control (1C, sample 2.3).

BMP-2 showed only a slight effect on expression of the hedgehog receptor patched, since the relative expression level was increased only by factor 0.5 after BMP-2 treatment for 72 h (FIG. 1D, sample 1.1). There was no increase after 11 days (sample 2.1). The combination of BMP-2 and Shh resulted in ptc mRNA increase of about 3-fold at both time points tested (samples 1.3 and 2.3). Patched expression was upregulated 2.5-fold by Shh alone at both time points (samples 1.2 and 2.2).

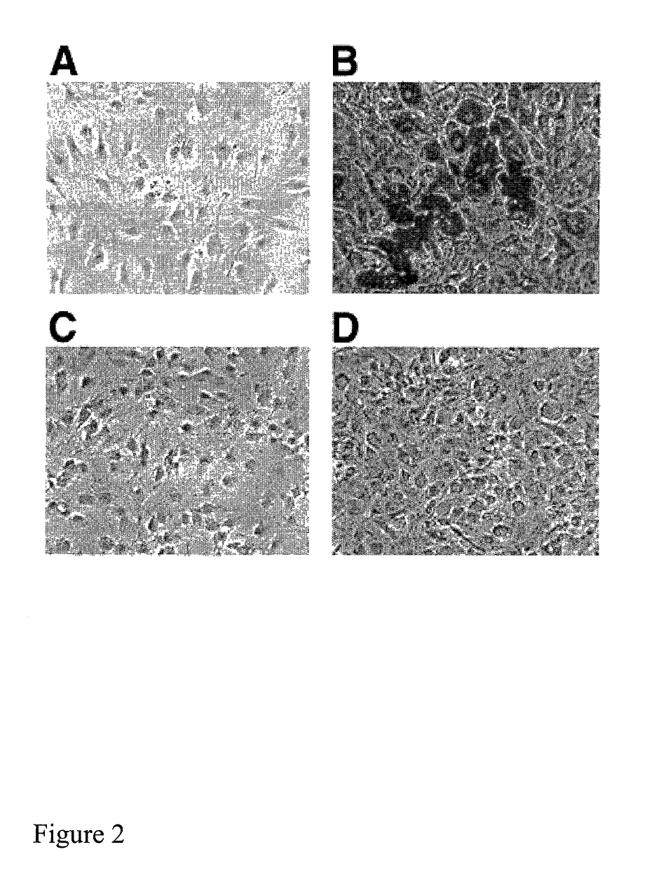

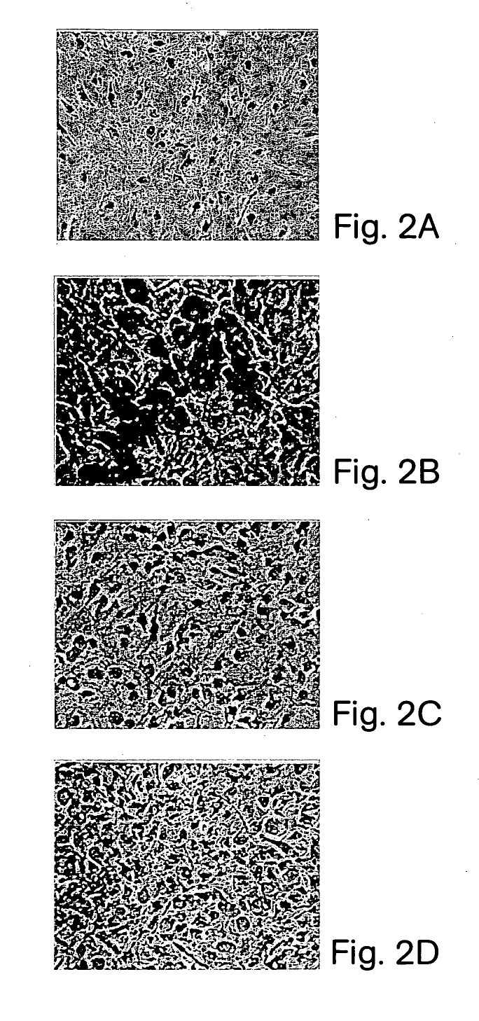

In order to confirm the molecular status of the cells, we examined the cellular phenotype by adipocyte specific cytological staining. After 11 days of BMP-2 treatment a significant amount of cells acquired the rounded shape of mature adipocytes (FIG. 2B). Oil Red O staining demonstrated that the cytosol of these cells contained numerous lipid-filled vesicles. Untreated control cells showed no significant Oil Red O staining after this time period (FIG. 2A). Interestingly the cells treated with Shh alone (FIG. 2C) and with a combination of BMP-2 and Shh showed neither lipid vesicles nor Oil Red O staining (FIG. 2D). This points to a suppressing effect of Shh on BMP-2 induced adipocytic differentiation. No adipocytes were visible in any of the 72 h cultures (data not shown).

References

AHRENS, M., ANKENBAUER, T., SCHRÖDER, D., HOLLNAGEL, A., MAYER, H. and GROSS, G. (1993). Expression of humen bone morphogenetic protein-2 or -4 in murine mesenchymal progenitor C3H10T1/2 cells induces differentiation into distinct mesenchymal cell lineages. DNA Cell. Biol. 12, 871-880.

BENNETT, J. H., JOYNER, C. J., TRIFFITT, J. T. and OWEN, M. E. (1991). Adipocytic cells cultured from marrow have osteogenic potential. J. Cell. Sci. 99, 131-139.

BERESFORD, J. N., BENNETT, J. H., DEVLIN, C., LEBOY, P. S. and OWEN, M. E. (1992). Evidence for an inverse relationship between the differentiation of adipocytic and osteogenic cells in rat marrow stromal cell cultures. J. Cell. Sci. 102, 341-351.

BRUDER, S. P., FINK, D. J. and CAPLAN, A. I. (1994). Mesenchymal Stem Cells in Bone Development, Bone Repair, and Skeletal Regeneration Therapy. J. Cell. Biochem. 56, 283-294.

DIASCRO, D. D., VOGEL, R. L., JOHNSON, T. E., WITHERUP, K. M., PITZENBERGER, S. M., RUTLEDGE, S. J., PRESCOTT, D. J., RODAN, G. A. and SCHMIDT, A. (1998). High fatty acid content in rabbit serum is responsible for the differentiation of osteoblasts into adipocyte-like cells. J. Bone Miner. Res. 13, 96-106.

ELBRECHT, A., CHEN, Y., CULLINAN, C. A., HAYES, N., LEIBOWITZ, M. D., MOLLER, D. E. and BERGER, J. (1996). Molecular Cloning, Expression and Characterization of Human Peroxisome Proliferator Activated Receptors y1 and y2. Biochem. Biophys. Res. Com. 224,431-437.

GILLILAND, G., PERRIN, S., BLANCHARD, K. and BUNN, H. F. (1990). Analysis of cytokine mRNA and DNA: detection and quantitation by competitive polymerase chain reaction. Proc. Natl. Acad. Sci. USA 87, 2725-2729.

KATAGIRI, T., YAMAGUCHI, A., IKEDA, T., YOSHIKI, S., WOZNEY, J. M., ROSEN, V., WANG, E. A., TANAKA, H., OMURA, S. and SUDA, T. (1990). The non-osteogenic mouse pluripotent cell line, C3H10T1/2, is induced to differentiate into osteoblastic cells by recombinant human bone morphogenetic protein-2. Biochem. Biophys. Res. Commun. 172, 295-299.

KAWABATA, M., IMAMURA, T. and MIYAZONO, K. (1998). Signal transduction by bone morphogenetic proteins. Cytokine Growth Factor Rev. 9, 49-61.

KINTO, N., IWAMOTO, M., ENOMOTO-IWAMOTO, M., NOJI, S., OHUCHI, H., YOSHIOKA, H., KATAOKA, H., WADA, Y., YUHAO, G., TAKAHASHI, H. E., YOSHIKI, S. and YAMAGUCHI, A. (1997). Fibroblasts expressing Sonic hedgehog induce osteoblast differentiation and ectopic bone formation. FEBS Lett 404, 319-323.

LAUFER, E., NELSON, C. E., JOHNSON, R. L., MARGAN, B. A. and TABIN, C. (1994). Sonic hedgehog and Fgf-4 act through a signaling cascade and feedback loop to integrate growth and patterning of the developing limb bud. Cell 79, 993-1003.

MARIGO, V., DAVEY, R. A., ZUO, Y., CUNNINGHAM, J. M. and TABIN, C. J. (1996). Biochemical evidence that Patched is the Hedgehog receptor. Nature 384, 176-179.

MATARESE, V. and BERNLOHR, D. A. (1988). Purification of murine adipocyte lipid-binding protein. J. Biol. Chem. 263, 14544-14551.

NAKAMURA, T., AIKAWA, T., IWAMOTO-ENOMOTO, M., IWAMOTO, M., HIGUCHI, Y., PACIFICI, M., KINTO, N., YAMAGUCHI, A., NOJI, S., KURISU, K. and MATSUYA, T. (1997). Induction of osteogenic differentiation by hedgehog proteins. Biochem. Biophys. Res. Commun. 237, 465-469.

PLATT, K. A., MICHAUD, J. and JOYNER, A. L. (1997). Expression of the mouse Gli and Ptc genes is adjacent to embryonic sources of hedgehog signals suggesting a conservation of pathways between flies and mice. Mech. Dev. 62, 121-135.

TONTONOZ, P., HU, E., GRAVES, R. A., BUDAVARI, A. I. and SPIEGELMAN, B. M. (1994). mPPARy2: tissue-specific regulator of an adipocyte enhancer. Genes Dev. 8, 1224-1234.

WANG, E. A., ISRAEL, D. I., KELLY, S. and LUXENBERG, D. P. (1993). Bone morphogenetic protein-2 causes commitment and differentiation in C3H10T1/2 and 3T3 cells. Growth Factors 9, 57-71.

WANG, E. A., ROSEN, V., CORDES, P., HEWICK, R. M., KRIZ, M. J., LUXENBERG, D. P., SIBLEY, B. S. and WOZNEY, J. M. (1988). Purification and characterization of other distinct bone-inducing factors. Proc. Natl. Acad. Sci. USA 85, 9484-9488.

ZEHENTNER, B. K., DONY, C. and BURTSCHER, H. (1999). The transcription factor Sox9 is involved in BMP-2 signaling. J. Bone Miner. Res. 14, 1733-1740.

All of the above-cited references and publications are hereby incorporated by reference.

### Equivalents

Those skilled in the art will recognize, or be able to ascertain using no more than routine experimentation, many equivalents to the specific embodiments of the invention described herein. Such equivalents are intended to be encompassed by the following claims.

We claim:

1. A method for regulating formation and/or maintenance of adipocyte tissue comprising contacting pre-adipocyte or adipocyte cells with a hedgehog polypeptide or a ptc therapeutic.

2. The method of claim 1, wherein the hedgehog polypeptide is modified with one or more lipophilic moieties.

3. The method of claim 2, wherein the hedgehog polypeptide is modified with one or more serol moieties.

4. The method of claim 2, wherein the sterol moiety is cholesterol.

5. The method of claim 2, wherein the hedgehog polypeptide is modified with one or more fatty acid moieties.

6. The method of claim 5, wherein each fatty acid moiety is independently selected from the group consisting of myristoyl, palmitoyl, stearoyl, and arachidoyl.

7. The method of claim 2, wherein the hedgehog polypeptide is modified with one or more aromatic hydrocarbons.

8. The method of claim 1, wherein the ptc therapeutic binds to patched and mimics hedgehog-mediated patched signal transduction.

9. The method of claim 8, wherein the ptc therapeutic is a small organic molecule.

10. The method of claim 8, wherein the binding of the ptc therapeutic to patched results in upregulation of patched and/or gli expression.

11. The method of claim 1, wherein the ptc therapeutic is a small organic molecule which interacts with neuronal cells to mimic hedgehog-mediated patched signal transduction.

12. The method of claim 1, wherein the ptc therapeutic mimics hedgehog-mediated patched signal transduction by altering the localization, protein-protein binding and/or enzymatic activity of an intracellular protein involved in a patched signal pathway.

13. The method of claim 1, wherein the ptc therapeutic alters the level of expression of a hedgehog protein, a patched protein or a protein involved in the intracellular signal transduction pathway of patched.

14. The method of claim 13, wherein the ptc therapeutic is an antisense construct which inhibits the expression of a protein which is involved in the signal transduction pathway of patched and the expression of which antagonizes hedgehog-mediated signals.

15. The method of claim 14, wherein the antisense construct is an oligonucleotide of about 20-30 nucleotides in length and having a GC content of at least 50 percent.

16. The method of claim 15, wherein the antisense oligonucleotide is selected from the group consisting of:
5′-GTCCTGGCGCCGCCGCCGCCGTCGCC;
5′-TTCCGATGACCGGCCTTTCGCGGTGA; and
5′-GTGCACGGAAAGGTGCAGGCCACACT

17. The method of claims 13, wherein the ptc therapeutic is a small organic molecule which binds to patched and regulates patched-dependent gene expression.

18. The method of claim 12, wherein the ptc therapeutic is an inhibitor of protein kinase A.

19. The method of claim 18, wherein the PKA inhibitor is a 5-isoquinolinesulfonamide

20. The method of claim 19, wherein the PKA inhibitor is represented in the general formula: 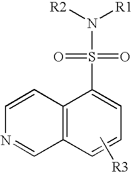
wherein,
R₁ and R₂ each can independently represent hydrogen, and as valence and stability permit a lower alkyl, a lower alkenyl, a lower alkynyl, a carbonyl (such as a carboxyl, an ester, a formate, or a ketone), a thiocarbonyl (such as a thioester, a thioacetate, or a thioformate), an amino, an acylamino, an amido, a cyano, a nitro, an azido, a sulfate, a sulfonate, a sulfonamido, —(CH₂)ₘ—R₈, —(CH₂)ₘ—OH, —(CH₂)ₘ—O-lower alkyl, —(CH₂)ₘ—O-lower alkenyl, —(CH₂)ₙ—O—(CH₂)ₘ—R₈, —(CH₂)ₘ—SH, —(CH₂)ₘ—S-lower alkyl, —(CH₂)ₘ—S-lower alkenyl, —(CH₂)ₙ—S—(CH₂)ₘ—R₈, or
R₁ and R₂ taken together with N form a heterocycle (substituted or unsubstituted);
R₃ is absent or represents one or more substitutions to the isoquinoline ring such as a lower alkyl, a lower alkenyl, a lower alkynyl, a carbonyl (such as a carboxyl, an ester, a formate, or a ketone), a thiocarbonyl (such as a thioester, a thioacetate, or a thioformate), an amino, an acylamino, an amido, a cyano, a nitro, an azido, a sulfate, a sulfonate, a sulfonamido, —(CH₂)ₘ—R₈, —(CH₂)ₘ—OH, —(CH₂)ₘ—O-lower alkyl, —(CH₂)ₘ—O-lower alkenyl, —(CH₂)ₙ—O—(CH₂)ₘ—R₈, —(CH₂)ₘ—SH, —(CH₂)ₘ—S-lower alkyl, —(CH₂)ₘ—S-lower alkenyl, —(CH₂)ₙ—S—(CH₂)ₘ—R₈;
R₈ represents a substituted or unsubstituted aryl, aralkyl, cycloalkyl, cycloalkenyl, or heterocycle; and
n and m are independently for each occurrence zero or an integer in the range of 1 to 6.

21. The method of claim 18, wherein the PKA inhibitor is cyclic AMP analog.

22. The method of claim 18, wherein the PKA inhibitor is selected from the group consisting of N-[2-((p-bromocinnamyl)amino)ethyl]-5-isoquinolinesulfonamide, 1-(5-isoquinolinesulfonyl)-2-methylpiperazine, KT5720, 8-bromo-cAMP, dibutyryl-cAMP and PKA Heat Stable Inhibitor isoform.

23. The method of claim 1, wherein patient is being treated prophylactically.

24. A therapeutic preparation of a small molecule antagonist of patched, which patched antagonist is provided in a pharmaceutically acceptable carrier and in an amount sufficient to regulate growth and/or maintenance of adipocyte cells.

25. A method for regulating the growth state of a adipocyte stem/progenitor cell comprising contacting the cell with a hedgehog polypeptide or a ptc therapeutic.

26. A method for treatment or prevention of disorders of, or surgical or cosmetic repair of, such adipocyte tissues, comprising administering to the patient a hedgehog polypeptide or a ptc therapeutic.
